(19) United States
(12) Patent Application Publication
(10) Pub. No.: US 2026/0347876 A1
(43) Pub. Date: Nov. 5, 2026

(54) THIRD-GENERATION LENTIVIRAL GENE THERAPY RESCUES FUNCTION IN A MOUSE MODEL OF USHER 1B

(71) Applicants: University of Kansas, Lawrence, KS (US); Medizinische Hochschule Hannover, Hannover (DE)

(72) Inventors: Hinrich STAECKER, Leawood, KS (US); Axel SCHAMBACH, Hannover (DE); Michael MORGAN, Hannover (DE); Juliane SCHOTT, Hannover (DE); Hildegard BÜNING, Hannover (DE); Athanasia WARNECKE, Hannover (DE)

(73) Assignees: University of Kansas, Lawrence, KS (US); Medizinische Hochschule Hannover, Hannover (DE)

(21) Appl. No.: 19/120,488

(22) PCT Filed: Oct. 12, 2023
(86) PCT No.: PCT/US2023/035014

§ 371 Date: Apr. 11, 2025

Related U.S. Application Data
(60) Provisional application No. 63/415,612, filed on Oct. 12, 2022.

(51) Int. Cl. C12N 15/86; A61K 38/00; A61K 48/00; A61P 27/16; C07K 14/47

(52) U.S. Cl. CPC C12N 15/86; A61K 48/005; A61K 48/0075; A61P 27/16; C07K 14/4716; A61K 38/00; C12N 2740/15022; C12N 2740/15043; C12N 2740/15045; C12N 2740/16022; C12N 2740/16043; C12N 2740/16045; C12N 2800/40; C12N 2830/48; C12N 2840/203

(57) ABSTRACT

The present disclosure provides third-generation self-inactivating (SIN) lentiviral vectors comprising a nucleic acid sequence encoding Myo VIIa isoform 1 for treatment of Usher IB syndrome in a subject in need thereof. In some embodiments, treatment of Usher IB syndrome comprises amelioration of presbycusis caused by heterozygous mutations in Myo VIIa and/or balance problems caused by homozygous mutations in Myo VIIa.

### CROSS-REFERENCE TO RELATED APPLICATIONS

This application claims the benefit of and priority to U.S. Provisional Patent Application No. 63/415,612, filed Oct. 12, 2022, the entire contents of which are incorporated herein by reference.

### TECHNICAL FIELD

The present technology relates generally to third-generation self-inactivating (SIN) lentiviral vectors comprising a nucleic acid sequence encoding MyoVIIa isoform 1 for treatment of Usher 1B syndrome in a subject in need thereof. In some embodiments, treatment of Usher 1B syndrome comprises amelioration of presbycusis (age-related hearing loss) caused by heterozygous mutations in MyoVIIa and/or balance problems caused by homozygous mutations in MyoVIIa.

### BACKGROUND

The following description of the background of the present technology is provided simply as an aid in understanding the present technology and is not admitted to describe or constitute prior art to the present technology.

Usher syndrome 1B is a devastating genetic disorder with congenital deafness, loss of vestibular function and blindness caused by mutations in the myosin VII (MYO7A) gene. The auditory-vestibular deficits of this disorder can be modelled in Shaker-1 mice, which develop hearing and balance loss after postnatal day 14. Heterozygous animals were found to have normal hearing and vestibular function until 6 months of age, at which point they developed severe hearing loss across all frequencies.

Due to the genetic nature of USH1B, gene therapy is a promising novel treatment option. However, significant challenges in developing gene therapy for this disorder include age at hearing loss onset and the size of the transgene. Currently, adeno-associated viral (AAV) vectors are the most widely explored strategy for gene therapy in the inner ear. However, myosin VIIA is too large to be delivered with standard AAV technology. Although split AAV strategies have been deployed in the eye, these were associated with a loss in efficiency [10]. In contrast, lentiviral vectors have a high coding capacity of up to and beyond 10 kb, allowing the delivery of the whole coding sequence of large genes as well as of a range of regulatory sequences without the loss of efficiency as associated with a split AAV strategy [10]. Lentiviral vectors traditionally have not been efficient in transducing cells of the inner ear [11,12]. See also Han et al., HUMAN GENE THERAPY 10:1867-1873 (1999) (demonstrating poor in vivo distribution of lentiviral vectors in cochlea).

### SUMMARY

In one aspect, the present disclosure provides a method for treating or preventing Usher 1B syndrome in a patient in need thereof comprising administering to the patient an effective amount of a composition comprising a self-inactivating (SIN) lentiviral expression vector pseudotyped with a viral envelope glycoprotein that is configured to bind to a receptor expressed in an inner ear cell, wherein the SIN lentiviral expression vector comprises: a 5′ long terminal repeat (LTR) region comprising or consisting of a constitutively active or inducible heterologous promoter sequence and the Repeat (R)-U5 sequence of SEQ ID NO: 4, wherein the constitutively active heterologous promoter sequence is located upstream of the R-U5 sequence; a 5′ untranslated region (UTR) comprising the splice donor (SD) sequence of SEQ ID NO: 5, a packaging signal sequence, a Rev-responsive element (RRE), the splice acceptor (SA) sequence of SEQ ID NO: 9, and optionally, a polypurine tract (PPT) region; an internal promoter operably linked to a cargo sequence, wherein the cargo sequence comprises a gene sequence encoding MyoVIIa isoform 1, and optionally a gene sequence encoding a reporter protein; RNA processing elements comprising a posttranscriptional regulatory element (PRE), wherein the PRE is located downstream of the cargo sequence, and a 3′ LTR region comprising or consisting of the deleted U3-R sequence of SEQ ID NO: 11, wherein the 3′ LTR region is located downstream of the PRE, and wherein the SIN lentiviral expression vector does not comprise a PPT region between the cargo sequence and the PRE. In some embodiments, the 5′ UTR further comprises a primer binding site sequence, optionally wherein the primer binding site sequence comprises SEQ ID NO: 13. Additionally or alternatively, in some embodiments, the gene sequence encoding MyoVIIa isoform 1 comprises SEQ ID NO: 1. In any of the preceding embodiments, the PRE comprises SEQ ID NO: 10. The SIN lentiviral expression vector may be derived from a lentivirus selected from the group consisting of human immunodeficiency virus (e.g., HIV-1, HIV-2), simian immunodeficiency virus (SIV), feline immunodeficiency virus (FIV), and equine infectious anemia virus (EIAV). Additionally or alternatively, the SIN lentiviral expression vector lacks vif, vpr, vpu, nef, and optionally tat genes and/or wherein the SIN lentiviral expression vector comprises a PPT region of SEQ ID NO: 41 between the PRE and the 3′ LTR region.

Additionally or alternatively, in some embodiments, the packaging signal sequence comprises SEQ ID NO: 6 and/or the RRE comprises SEQ ID NO: 7. In some embodiments, the PPT region of the 5′ UTR comprises SEQ ID NO: 8 and/or is located downstream of the RRE.

In some embodiments, the constitutively active heterologous promoter sequence is a Rous sarcoma virus (RSV) promoter sequence, a human cytomegalovirus (CMV) promoter sequence, an HIV promoter sequence, a eukaryotic promoter sequence (e.g., EF1a, PGK1, UBC, or human beta actin). In certain embodiments, the RSV promoter sequence comprises SEQ ID NO: 3. In other embodiments, the inducible heterologous promoter sequence is a tetracycline response element (TRE) that can be bound by a tetracycline transactivator (tTA) protein in the presence of tetracycline or an analogue thereof, e.g. doxycycline.

In some embodiments, the internal promoter is selected from the group comprising a promoter derived from CMV, spleen focus-forming virus (SFFV), myeloproliferative sarcoma virus (MPSV), murine embryonal stem cell virus (MESV), murine leukemia virus (MLV) and simian virus 40 (SV40). In another embodiment, the internal promoter may be a CAG promoter, i.e. a composite construct consisting of the CMV enhancer fused to the chicken beta-actin promoter and the rabbit beta-Globin splice acceptor site. Additionally or alternatively, in certain embodiments, the SFFV promoter sequence comprises SEQ ID NO: 2. In other embodiments, the CAG promoter comprises SEQ ID NO: 18.

In some embodiments, an IRES is interspersed between the gene sequence encoding MyoVIIa isoform 1, and the gene sequence encoding a reporter protein, optionally wherein the IRES comprises SEQ ID NO: 15. Examples of reporter proteins include, but are not limited to dTomato, TagBFP, Azurite, EBFP2, mKalama1, Sirius, Sapphire, T-Sapphire, ECFP, Cerulean, SCFP3A, mTurquoise, monomeric Midoriishi-Cyan, TagCFP, mTFP1, EGFP, Emerald, Superfolder GFP, Monomeric Azami Green, TagGFP2, mUKG, mWasabi, EYFP, Citrine, Venus, SYFP2, TagYFP, Monomeric Kusabira-Orange, mKOK, mKO2, mOrange, mOrange2, mRaspberry, mCherry, dsRed, mStrawberry, mTangerine, tdTomato, TagRFP, TagRFP-T, mApple, mRuby, mPlum, HcRed-Tandem, mKate2, mNeptune, NirFP, TagRFP657, IFP1.4, iRFP, mKeima Red, LSS-mKate1, LSS-mKate2, PA-GFP, PAmCherry 1, PATagRFP, Kaede (green), Kaede (red), KikGR1 (green), KikGR1 (red), PS-CFP2, PS-CFP2, mEos2 (green), mEos2 (red), PSmOrange, and Dronpa

Additionally or alternatively, the 5′ UTR comprises a mutant group-specific antigen (Gag) sequence. In one embodiment, the mutant Gag sequence comprises SEQ ID NO: 14.

In some embodiments, the SIN lentiviral vector is pseudotyped with a viral envelope glycoprotein capable of binding to a receptor selected from the group consisting of the LDL-receptor and LDL-R family members, the SLC1A5-receptor, the Pit1/2-receptor and the PIRYV-G-receptor. The viral envelope glycoprotein capable of binding the LDL-receptor or LDL-R family members may be, e.g., MARAV-G, COCV-G, VSV-G or VSV-G ts.

In any and all embodiments of the methods disclosed herein, the patient is heterozygous or homozygous for a MYO7A genetic mutation. The patient may be diagnosed with Usher syndrome type IB. Accordingly, the SIN lentiviral composition of the present technology may be suitable to effectively improve or even eliminate presbycusis or balance dysfuntion in the patient.

Additionally or alternatively, in some embodiments, the methods of the present technology comprise administering to the patient a second vector comprising gag and pol, and optionally tat, a third plasmid comprising the viral envelope glycoprotein, and a fourth vector comprising regulatory gene Rev.

In any of the preceding embodiments, the methods of the present technology further comprise separately, sequentially or simultaneously administering one or more additional therapeutic agents to the patient. The composition of the present technology may be administered to the patient daily, weekly, biweekly, every 3 weeks, every 4 weeks, monthly, or annually. Additionally or alternatively, in some embodiments, the composition is administered via a cochlea implant route, round window injection, oval window injection, canalostomy, cochleostomy or injection into the endolymphatic sac. In some embodiments, the composition has a titer of at least 7.78×10⁶ TU/mL. The titer of the composition may be concentrated using ultracentifugation, microfiltration, ultrafiltration, chromatography, density gradient ultracentrifugation, tangential flow microfiltration, or precipitation.

In any and all embodiments of the methods disclosed herein, the inner ear cell is selected from the group consisting of cells of the organ of Corti including supporting cells as well as inner and outer hair cells, spiral ganglion neurons and glial cells, cells of the stria vascularis and spiral ligament, lateral wall fibrocytes, type I and II vestibular hair cells, vestibular supporting cells and vestibular ganglion neurons.

Additionally or alternatively, in some embodiments, the composition is administered after onset of balance dysfuntion in the patient.

### BRIEF DESCRIPTION OF THE DRAWINGS

FIGS. 1A-1E. Lentiviral vector-mediated packaging, delivery and expression of MYO7A is efficient and dose-controllable. FIG. 1A: Design of 3^{rd} generation self-inactivating (SIN) lentiviral vectors co-encoding for human myosin VIIA and a dTomato reporter protein (LV-MYO7A) or encoding for a dTomato reporter protein only (LV-ctrl). An internal spleen focus forming virus (SFFV) promoter was installed to drive transgene expression. R, repeat region; U5, 5′ unique region; SD, splice donor; y, packaging signal; RRE, rev-responsive element; cPPT, central polypurine tract; SA, splice acceptor; IRES, internal ribosomal entry site; PRE, post-transcriptional regulatory element; AU3, 3′ unique region with self-inactivating (SIN) deletion. FIG. 1B: Titers of viral vector preparations. Titer determination was based on transduction of HT1080 cells with different volumes of vector preparation and subsequent determination of the percentage of dTomato-expressing cells by flow cytometry. TU, transducing units. n=3-5 biological replicates. FIG. 1C: Percentage of HEI-OC1 cells expressing the LV vector-encoded dTomato reporter protein upon transduction with LV-MYO7A or LV-ctrl at a defined particle number per seeded cell (MOI). The percentage of dTomato-positive cells among the population of singlets within live cells was determined by flow cytometry. MOI, multiplicity of infection. Statistics: Multiple t tests; discovery determined using the Two-stage linear step-up procedure of 20 Benjamini, Krieger and Yekutieli, with Q=1%. FIG. 1D: Immunofluorescence analysis of dTomato and MYO7A expression in HEI-OC1 cells transduced with LV-MY07A or LV-ctrl at MOI of 0.2 and MOI of 2, and in non-transduced control cells (NTC). Staining for MYO7A was performed using an anti-MYO7A primary antibody and an AF488-conjugated secondary antibody. Scale bar: 200 μm. FIG. 1E: Percentage of HEI-OC1 cells expressing the vector-encoded dTomato reporter and the MYO7A protein. Flow cytometry analysis was performed upon intracellular staining for MYO7A expression in non-transduced controls (NTC) or cells transduced with LV-ctrl or LV-MYO7A at MOI 2. Samples were either incubated with secondary antibody only (unstained) or stained with an anti-MYO7A primary and an AF488-conjugated secondary antibody. The populations shown had been pre-gated for viable cells using SSC-A/FSC-A characteristics followed by gating for single cells according to FSC-A/FSC-H characteristics.

FIGS. 2A-2B. LV-MYO7A is expressed throughout the cochlea upon in-vivo administration and does not negatively impact normal hearing or balance in wild-type mice. Normal hearing, wild-type C57BL/6 mice were injected with LV-MYO7A through canalostomy using 1 μL of viral vector preparation. FIG. 2A: Rotarod times of the same cohort of mice before (pre) and after (post) canalostomy and LV-MYO7A administration. Sec, seconds. N=5. Statistics: Unpaired t test. FIG. 2B: ABR thresholds for four different frequencies (4, 8, 16 and 32 kHz) recorded for the same cohort of mice before (pre) and after (post) canalostomy and LV-MYO7A administration. Dotted lines show hearing thresholds for individual mice; solid lines show the mean thresholds from all mice at a given time point (pre or post). dB, decibel; Hz, Hertz. N=5. Statistics: Multiple t tests; discovery determined using the Two-stage linear step-up procedure of Benjamini, Krieger and Yekutieli, with Q=1%.

FIGS. 3A-3E. Homozygous mutant Shaker-1^{mut} mice show early-onset hearing and balance loss, heterozygous mutant Shaker-1^{het} mice show normal balance and late-onset hearing loss. Wild-type Shaker-1WT, heterozygous mutant Shaker-1^{het} and homozygous mutant Shaker-1^{mut} mice were evaluated for hearing and balance function using ABR, Rotarod testing and actimeter testing (FIGS. 3A-3C) for four different frequencies (4, 8, 16 and 32 kHz) recorded at P16, P21, 5 P30 and 6 months of age. dB, decibel; Hz, Hertz. FIG. 3A: Wild-type Shaker-1WT mice demonstrate normal hearing thresholds for the first 180 days of life. FIG. 3B: Mice heterozygous for the Shaker-1 mutation initially show normal ABR thresholds at P30 but develop a progressive hearing loss resulting in abnormal hearing thresholds at all frequencies by postnatal day 180. FIG. 3C: Mice homozygous for the Shaker-1 mutation demonstrated testable hearing at P16 with progression to profound hearing loss by P30. FIG. 3D: Balance was evaluated by testing the time these mice could remain on a rotarod at P30, P60, P90 and P270. Heterozygotes showed consistently normal rotarod times through P270. Shaker-1mut mice initially showed normal rotarod scores at P30. These rapidly declined and remained abnormal throughout the entire test period. There is a statistically significant difference between rotarod times at P270 when comparing heterozygotes and homozygous mutant mice and when comparing P30 to P270 Shaker-1mut mice. Sec, seconds. FIG. 3E: Force-plate actimeter testing was performed to determine the total distance traveled, the area covered during movement, and the total degree of right and left turns at P30, P60 and P90. Shaker-1mut mice demonstrated a progressive loss of balance function showing changes in total distance traveled, total area covered and the number of degrees of right and left turns (indicating circling behavior). Heterozygous Shaker-1^{het} mice showed normal values throughout P90. N=5.

FIGS. 4A-4F. LV-MYO7A gene therapy in Shaker-1^{mut} mice achieves transgene delivery to inner, outer and vestibular hair cells. FIGS. 4A-4C: Cochleae from untreated homozygous mutant Shaker-1^{mut} mice were evaluated at P30, P90 and 1 year of age. FIG. 4A: Histologic evaluation at P30 shows the presence of a normal appearing organ of Corti (bracket). FIG. 4B: By P90, immunofluorescent staining for annexin V shows labeling in inner (*) and outer (***) hair cells, as well as the supporting cells underneath the outer hair cell layer. Red signal: Annexin V; blue signal: nuclei stained with DAPI. FIG. 4C: By one year of age, the organ of Corti (bracket) has completely degenerated and the adjoining spiral ganglion is no longer seen (arrow). FIGS. 4D-4F: LV-MYO7A was delivered to Shaker-1^{mut} mice at P16. The cochleae from treated animals were evaluated two months after delivery. Red signal: dTomato; blue signal: nuclei stained with DAPI. FIG. 4D: Representative image from the basal turn of a cochlea from a treated animal. Expression of the dTomato marker gene can be seen in the inne hair cell (arrow). The adjoining outer hair cells already appear damaged at this point. FIG. 4E: Representative image from the apical turn of a cochlea from a treated animal. Both inner and outer hair cells (arrows) clearly express the dTomato marker, showing delivery of the construct to the appropriate target cells. FIG. 4F: Representative image from the vestibular organ of a treated animal. Vestibular hair cells also clearly express the dTomato marker gene (arrow).

FIGS. 5A-5G. LV-MYO7A gene therapy at P16 reduces hearing and balance loss in homozygous mutant Shaker-1^{mut} mice. Homozygous mutant Shaker-1^{mut} mice were treated with LV-MYO7A, delivered via the posterior semicircular canal at age P16. FIG. 5A: ABR thresholds for four different frequencies (4, 8, 16 and 32 kHz) in untreated and LV-MY07A-treated Shaker-1^{mut} mice at 2.5 months post-vector-delivery. dB, decibel; Hz, Hertz. N=12. FIG. 5B: Rotarod times determined at 1.5 (P60) and 2.5 months post-vector delivery (P90) in LV-MYO7A-treated Shaker-1^{mut} mice and at P90 in control Shaker-1^{het} and untreated Shaker-1^{mut} mice. Sec, seconds. N=7. FIG. 5C: Actimeter tracings were quantified to determine the total distance traveled, the area covered during movement, and the total degree of right and left turns at P60. There is a statistically significant improvement in actimeter scores in the LV-MY07A-treated animals. FIGS. 5D-5G: LV-MYO7A was delivered to Shaker-1^{mut} mice at P16. The cochleae from treated animals were evaluated two months after delivery. Red signal: dTomato; blue signal: nuclei stained with DAPI. FIG. 5D: Representative image from the basal turn of a cochlea from a treated animal. Expression of the dTomato marker gene can be seen in the inner hair cell (arrow). The adjoining outer hair cells already appear damaged at this point. FIG. 5E: Representative image from the apical turn of a cochlea from a treated animal. Both inner and outer hair cells (arrows) clearly express the dTomato marker, showing delivery of the construct to the appropriate target cells. FIG. 5F: Representative image from the vestibular organ of a treated animal. Vestibular hair cells also clearly express the dTomato marker gene (arrow). FIG. 5G: Delivery of LV-MYO7A did not alter the expression of Annexin V (green) in the organ of Corti. A surviving inner hair cell and single outer hair cell expressing dTom are shown by arrows

FIGS. 6A-6C: Actimetry demonstrates a restoration of more normal movement patterns after LV-MYO7A gene therapy. Force-plate actimetry tracings for three consecutive measurement frames (left to right) of control Shaker-1^{WT} mice (FIG. 6A), untreated homozygous mutant Shaker-1^{mut} mice (FIG. 6B) and LV-MYO7A-treated homozygous mutant Shaker-1^{mut} mice (FIG. 6C). Mutant animals show only circling behavior whereas mutants treated with LV-MYO7A demonstrate some circling but also purposeful movement similar to Shaker-1 ^{wt} animals.

FIGS. 7A-7C. LV-MYO7A gene therapy at P4 does not rescue hearing loss, but reduces balance loss in homozygous mutant Shaker-1^{mut} mice. To evaluate if better hearing outcomes could be achieved if MYO7A was delivered prior to onset of hearing, a subset of neonatal animals was treated with LV-MYO7A at P4. FIG. 7A: ABR thresholds for four different frequencies (4, 8, 16 and 32 kHz) recorded at P30 in untreated and LV-MYO7A-treated Shaker-1^{mut} animals. dB, decibel; Hz, Hertz. N=7. FIG. 7B: Rotarod times determined at P60 in control Shaker-1^{het} mice and in untreated versus LV-MYO7A-treated Shaker-1^{mut} animals. Sec, seconds. N=7. FIG. 7C: Force-plate actimetry tracings were quantified to determine the total distance travelled, the area covered during movement, and the total degree of right and left turns at P60 in control Shaker-1^{het}, in untreated Shaker-1^{mut} and in LV-MYO7A-treated Shaker-1^{mut} animals.

FIGS. 8A-8B. LV-MYO7A gene therapy at P4 prevents hearing loss in heterozygous mutant Shaker-1^{het} mice. To evaluate if late-onset hearing loss in heterozygous mutant Shaker-1^{het} mice can be rescued by LV-MYO7A gene therapy, neonatal animals were treated with LV-MYO7A through canalostomy at P4. FIG. 8A: ABR thresholds for four different frequencies (4, 8, 16 and 32 kHz) recorded at P120 in wild-type Shaker-1^{WT} controls and in untreated versus LV-MYO7A-treated Shaker-1^{het} mice. dB, decibel; Hz, Hertz. N=7. FIG. 8B: DPOAE measured in untreated Shaker-1het mice at 1 month of age, and in untreated versus LV-MYO7A-treated Shaker-1het mice at 6 months of age. dB, decibel; SPL, sound pressure level; fDP, distortion product frequency. N=5. There is a decline, but not complete loss, of DPOAE in heterozygous Shaker-1^{het} mice. Despite improvement in ABR threshold, DPOAE were not rescued by LV-MYO7A.

FIG. 9. LV-MYO7A gene therapy at P4 restores normal ABR waves in heterozygous mutant Shaker-1het mice. ABR waveforms recorded across four different frequencies (4, 8, 16, 32 kHz) in Shaker-1 mice at six months of age. Upper: untreated heterozygous Shaker-1^{het} mice. Middle: heterozygous Shaker-1^{het} mice treated with LV-MYO7A at P4. Lower: untreated control Shaker-1^{WT} mice. Arrows indicate the hearing threshold. The LV-MYO7A-treated heterozygotes and the wild-type mice appear to have similar thresholds and similar waveform morphology across all frequencies. Untreated heterozygous mice at six months of age show moderate to severe hearing loss across the frequency range.

FIG. 10. LV-MYO7A gene therapy at P4 does not restore normal DPOAE in heterozygous mutant Shaker-1^{het} mice. DPOAE measured in untreated Shaker-1^{het} and Shaker-1^{wt} mice at 1 and 6 months of age, and in untreated versus LV-MYO7A-treated Shaker-1^{het} mice at 6 months of age. dB, decibel; SPL, sound pressure level; fDP, distortion product frequency. Sound floor (blue) N=5. There is a decline but not complete loss of DPOAE in heterozygous Shaker-1^{het and} Shaker-1^{WT} mice. Despite improvement in ABR threshold, DPOAE were not rescued by LV-MYO7A.

FIGS. 11A-11F. LV-MYO7A is expressed throughout the cochlea upon in-vivo administration and does not negatively impact normal hearing or balance in wild-type mice. Normal hearing, wild-type C57BL/6 mice were injected with LV-dTom (11A-11B) or LV-MYO7A (11C-11D) through canalostomy using 1 μL of viral vector preparation. FIG. 11A: Expression of dTom can be seen in the spiral ganglion, inner and outer hair cells in multiple turns of the cochlea (P100). FIG. 11B: Whole mount preparation of a middle turn segment of the organ of Corti shows dTom expression in inner and outer hair cells colabelled with phalloidin-FITC. (DAPI=blue) (P30). FIG. 11C: Delivery of LV-MYO7A similarly demonstrated transfection of both inner and outer hair cells as well as spiral ganglion cells in multiple turns (P120). FIG. 11D: Expression of dtom was also noted in the vestibular neuroepithelium (saccule=s; utricle=u) (P120). FIG. 11E: ABR thresholds for four different frequencies (4, 8, 16 and 32 kHz) recorded for the same cohort of mice before (pre) and after (post) canalostomy and LV-MYO7A administration. Dotted lines show hearing thresholds for individual mice; solid lines show the mean thresholds from all mice at a given time point (pre or post). dB, decibel; Hz, Hertz. N=5. FIG. 11F: Rotarod times of the same cohort of mice before (pre) and after (post) canalostomy and LV-MYO7A administration. Sec, seconds. N=5. Scale bar A,C, D=25 μm; B=10 μm.

FIGS. 12A-12D: Vestibular hair cells are a target for LV-MYO7A gene therapy: Normal hearing, wild-type C57BL/6 mice were injected with LV-MYO7A through canalostomy using 1 μL of viral vector preparation. One week post vector delivery the utricles were removed and dTom expressing cells that demonstrated stereocilia (phalloidin labelling) were quantified (FIG. 12A). Expression of dTom can be seen distributed throughout the utricle (FIG. 12B). Vestibular hair cells were quantified in serially sectioned utricles from Shaker-1^{het} and untreated Shaker-1^{mut} mice. Both type I (FIG. 12C) and type II (FIG. 12D) cells did not show any significant decline over time. Treatment of Shaker-1^{mut} mice with LV-MYO7A did not alter type I or type II hair cell counts.

FIGS. 13A-13C show differences in hearing loss, rotarod, distance travelled, area and turns in Shaker-1^{het} and untreated Shaker-1^{mut} mice.

FIG. 14 shows differences in distance travelled, and area in Shaker-1^{het} and untreated Shaker-1^{mut} mice over time.

FIG. 15 shows differences in distance travelled, area and turns in Shaker-1^{het} untreated Shaker-1^{mut} mice, treated Shaker-1^{mut} mice (receiving treatment at postnatal day 21) and delayed treated Shaker-1^{mut} mice (receiving treatment at age 2 months). DT measurement 2 for delayed treated Shaker-1^{mut} mice is one month after administration (age 3 months).

FIGS. 16A-16E. A lentiviral reporter vector efficiently transduces the in vivo mouse cochlea. Normal hearing, wild-type C57BL/6 mice were injected with 1 μL of LV-ctrl via the PSCC at one month of age. FIG. 16A: Representative whole cleared cochlea at one week post-injection of LV-ctrl. A 3D view was reconstructed in Nikon software from serial images taken under a confocal microscope. dTomato signal is seen throughout the inner ear (vestibular organ and cochlea), with distribution in the cochlea from base to apex. Asterisk (*)—organ of Corti; SG—spiral ganglion; VG—vestibular ganglion. Gray signal: dTomato. FIG. 16B: 3D image of a non-injected control cochlea. The individual images for 3D reconstruction in the dTomato channel were acquired with maximum gain, showing some autofluorescence in the lateral wall only. Scale bar: 500 μm (FIGS. 16A-16B). FIG. 16C High power confocal image showing the organ of Corti from the 3D image shown in FIG. 11A (right). Arrow-inner hair cell; bracket-outer hair cells. White signal: dTomato. (FIGS. 16D-16E) 4.1-fold magnification of the two regions from the cross section shown in FIG. 11B that contained organ of Corti tissue. Arrow-inner hair cell; bracket-outer hair cells. Red signal: dTomato.

FIGS. 17A-17D. LV-MYO7A transduces inner and outer hair cells in the in vivo cochlea. FIGS. 17A-17C: Representative cochlea sections from three independent wild-type C57BL/6 mice injected with 1 μL of LV-MYO7A via the PSCC. Insets: high power images of the organ of Corti region indicating dTomato signal (gray) in inner and outer hair cells. FIGS. 17A-17B: LV-MYO7A with CAGs promoter. FIG. 17C: LV-MYO7A with SFFV promoter. Scale bar: 48 μm. FIG. 17D: Representative cochlea section from a non-injected C57BL/6 control mouse. The asterisk (*) with bracket indicates the region of the organ of Corti. No dTomato signal is seen in inner and outer hair cells. Red signal: dTomato; blue signal: nuclei stained with DAPI. Scale bar: 42 μm.

FIGS. 18A-18P: LV-MYO7A efficiently transduces cochlear hair cells. FIGS. 18A-18D: Higher magnification (1.6-fold) of the inner hair cell region from the z-series cochlear whole mount images shown in FIG. 11D. Stereocilia labeled by Phalloidin-FITC identify hair cells, which are positive for dTomato. FIGS. 18E-18H: Merge and individual channels for the cochlear whole mount image shown in FIG. 11D (bottom). dTomato signal is seen throughout the inner and outer hair cell layers (FIG. 18F). Scale bar: 28 μm. FIGS. 181-18L: Representative z-series of images from a cochlear whole mount of a non-injected wild-type C57BL/6 control mouse. FIGS. 18M-18P: Merge and individual channels from the image shown in FIG. 18L. In the dTomato channel, some autofluorescence/spillover of signal from the FITC channel is visible in pillar cells; no signal is seen in inner hair cells (FIG. 18N). Arrow-inner hair cell row; bracket-outer hair cell rows. Red signal: dTomato; green signal: Phalloidin-FITC; blue signal: DAPI. Scale bar: 47 μm.

FIG. 19. LV-MYO7A gene therapy decelerates, but does not prevent hair cell degeneration in homozygous Shaker-1″ “ ” mice. Semi-quantitative evaluation of the presence of sensory hair cells in the cochlea. The percentage of inner (IHC) and outer hair cells (OHC) in the apical and basal turn of cochleae from untreated or LV-MYO7A treated Shaker-1^{WT} and Shaker-1^{mut} mice was determined. Midmodiolar sections were prepared at the indicated animal age, and the number of intact hair cells counted for five sections per animal. The percentage shown in the table for each group and ear was calculated as the number of intact hair cells found, divided by the expected number, and multiplied by factor 100. Treated animals had been injected into the left ear with 1 μL of LV.MYO7A (titer: 7.78×10⁶ TU/mL) at P16, while the respective right ear served as an untreated control. N=5 animals per group.

FIG. 20. Exemplary DNA and amino acid sequences of viral envelope glycoproteins for pseudotyping the SIN vectors disclosed herein.

### DETAILED DESCRIPTION

It is to be appreciated that certain aspects, modes, embodiments, variations and features of the present methods are described below in various levels of detail in order to provide a substantial understanding of the present technology.

In practicing the present methods, many conventional techniques in molecular biology, protein biochemistry, cell biology, immunology, microbiology and recombinant DNA are used. See, e.g., Sambrook and Russell eds. (2001) Molecular Cloning: A Laboratory Manual, 3^{rd} edition; the series Ausubel et al. eds. (2007) Current Protocols in Molecular Biology; the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5th edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); and Herzenberg et al. eds (1996) Weir's Handbook of Experimental Immunology. Methods to detect and measure levels of polypeptide gene expression products (i.e., gene translation level) are well-known in the art and include the use of polypeptide detection methods such as antibody detection and quantification techniques. (See also, Strachan & Read, Human Molecular Genetics, Second Edition. (John Wiley and Sons, Inc., NY, 1999)).

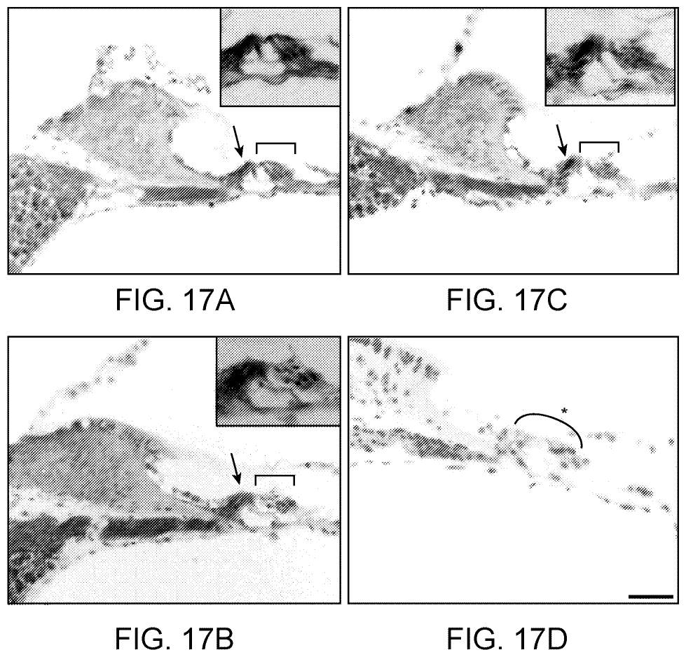

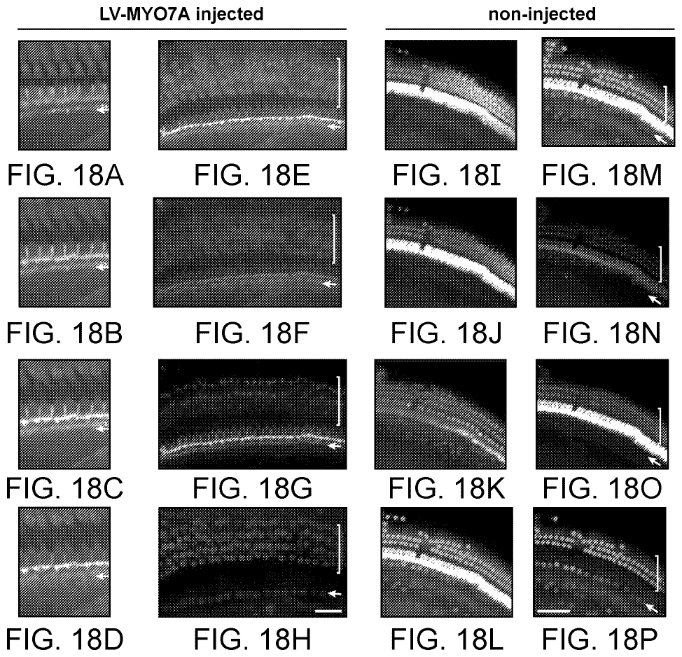

Disclosed herein is a novel third-generation, high-capacity lentiviral vector system for delivering the 6645 bp MYO7A gene in a single vector. The high-capacity lentiviral vector system of the present techology successfully transduced USH1B-relevant target cells and effectively ameliorated inner ear-related and balance-related deficits in Usher 1B subjects at a low titer of 7.78×10⁶ TU/mL (FIG. 11C and FIGS. 17A-17D). Delivery of MY07A at postnatal day 17 partially halted hearing loss progression and strongly reduced balance dysfunction in the Shaker-1^{mut} mouse model. MYO7A delivery prior to the onset of hearing did not improve hearing outcomes, but reduced circling behavior. In heterozygous mice, lentiviral MYO7A gene therapy halted hearing loss progression and achieved hearing thresholds as in wild-type littermates. As shown in FIG. 15, reversal of balance defects was also observed when Shaker-1^{mut} mice received the LV-MYO7A gene therapy at a significantly delayed timepoint (e.g., at age 2 months).

### DEFINITIONS

The definitions of certain terms as used in this specification are provided below. Unless defined otherwise, all technical and scientific terms used herein generally have the same meaning as commonly understood by one of ordinary skill in the art to which this present technology belongs. As used in this specification and the appended claims, the singular forms “a”, “an” and “the” include plural referents unless the content clearly dictates otherwise. For example, reference to “a cell” includes a combination of two or more cells, and the like. Generally, the nomenclature used herein and the laboratory procedures in cell culture, molecular genetics, organic chemistry, analytical chemistry and nucleic acid chemistry and hybridization described below are those well-known and commonly employed in the art.

As used herein, the term “about” in reference to a number is generally taken to include numbers that fall within a range of 1%, 5%, or 10% in either direction (greater than or less than) of the number unless otherwise stated or otherwise evident from the context (except where such number would be less than 0% or exceed 100% of a possible value).

As used herein, the “administration” of an agent or drug to a subject includes any route of introducing or delivering to a subject a compound to perform its intended function. Administration can be carried out by any suitable route, including but not limited to, orally, intranasally, parenterally (intravenously, intramuscularly, intraperitoneally, or subcutaneously), intrathecally, or topically. Administration includes self-administration and the administration by another.

As used herein, the term “biological sample” means sample material derived from living cells. Biological samples may include tissues, cells, protein or membrane extracts of cells, and biological fluids (e.g., ascites fluid or cerebrospinal fluid (CSF)) isolated from a subject, as well as tissues, cells and fluids present within a subject. Biological samples of the present technology include, but are not limited to, samples taken from breast tissue, renal tissue, the uterine cervix, the endometrium, the head or neck, the gallbladder, parotid tissue, the prostate, the brain, the pituitary gland, kidney tissue, muscle, the esophagus, the stomach, the small intestine, the colon, the liver, the spleen, the pancreas, thyroid tissue, heart tissue, lung tissue, the bladder, adipose tissue, lymph node tissue, the uterus, ovarian tissue, adrenal tissue, testis tissue, the tonsils, thymus, blood, hair, buccal, skin, serum, plasma, CSF, semen, prostate fluid, seminal fluid, urine, feces, sweat, saliva, sputum, mucus, bone marrow, lymph, and tears. Biological samples can also be obtained from biopsies of internal organs. Biological samples can be obtained from subjects for diagnosis or research or can be obtained from non-diseased individuals, as controls or for basic research.

As used herein, a “control” is an alternative sample used in an experiment for comparison purpose. A control can be “positive” or “negative.” For example, where the purpose of the experiment is to determine a correlation of the efficacy of a therapeutic agent for the treatment for a particular type of disease, a positive control (a compound or composition known to exhibit the desired therapeutic effect) and a negative control (a subject or a sample that does not receive the therapy or receives a placebo) are typically employed.

As used herein, the term “effective amount” refers to a quantity sufficient to achieve a desired therapeutic and/or prophylactic effect, e.g., an amount which results in the prevention of, or a decrease in a disease or condition described herein or one or more signs or symptoms associated with a disease or condition described herein. In the context of therapeutic or prophylactic applications, the amount of a composition administered to the subject will vary depending on the composition, the degree, type, and severity of the disease and on the characteristics of the individual, such as general health, age, sex, body weight and tolerance to drugs. The skilled artisan will be able to determine appropriate dosages depending on these and other factors. The compositions can also be administered in combination with one or more additional therapeutic compounds. In the methods described herein, the therapeutic compositions may be administered to a subject having one or more signs or symptoms of a disease or condition described herein. As used herein, a “therapeutically effective amount” of a composition refers to composition levels in which the physiological effects of a disease or condition are ameliorated or eliminated. A therapeutically effective amount can be given in one or more administrations.

As used herein, the term “expression” refers to the process by which polynucleotides are transcribed into mRNA and/or the process by which the transcribed mRNA is subsequently being translated into peptides, polypeptides, or proteins. If the polynucleotide is derived from genomic DNA, expression can include splicing of the mRNA in a eukaryotic cell. The expression level of a gene can be determined by measuring the amount of mRNA or protein in a cell or tissue sample. In one aspect, the expression level of a gene from one sample can be directly compared to the expression level of that gene from a control or reference sample. In another aspect, the expression level of a gene from one sample can be directly compared to the expression level of that gene from the same sample following administration of the compositions disclosed herein. The term “expression” also refers to one or more of the following events: (1) production of an RNA template from a DNA sequence (e.g., by transcription) within a cell; (2) processing of an RNA transcript (e.g, by splicing, editing, 5′ cap formation, and/or 3′ end formation) within a cell; (3) translation of an RNA sequence into a polypeptide or protein within a cell; (4) post-translational modification of a polypeptide or protein within a cell; (5) presentation of a polypeptide or protein on the cell surface; and (6) secretion or presentation or release of a polypeptide or protein from a cell.

As used herein, an “expression control sequence” refers to polynucleotide sequences which are necessary to affect the expression of coding sequences to which they are operably linked. Expression control sequences are sequences which control the transcription, post-transcriptional events and translation of nucleic acid sequences. Expression control sequences include appropriate transcription initiation, termination, promoter and enhancer sequences; efficient RNA processing signals such as splicing and polyadenylation signals; sequences that stabilize cytoplasmic mRNA; sequences that enhance translation efficiency (e.g., ribosome binding sites); sequences that enhance protein stability; and when desired, sequences that enhance protein secretion. The nature of such control sequences differs depending upon the host organism; in prokaryotes, such control sequences generally include promoter, ribosomal binding site, and transcription termination sequence. The term “control sequences” is intended to encompass, at a minimum, any component whose presence is essential for expression, and can also encompass an additional component whose presence is advantageous, for example, leader sequences.

As used herein, the term “gene” means a segment of DNA that contains all the information for the regulated biosynthesis of an RNA product, including promoters, exons, introns, and other untranslated regions that control expression.

As used herein, “heterologous nucleic acid sequence” is any sequence placed at a location in the genome where it does not normally occur. A heterologous nucleic acid sequence may comprise a sequence that does not naturally occur in a subject, or it may comprise only sequences naturally found in the subject, but placed at a non-normally occurring location in the genome, rendering it a heterologous sequence at that new site.

As used herein, a “host cell” is a cell that is used in to receive, maintain, reproduce and amplify a vector. A host cell also can be used to express the polypeptide encoded by the vector. The nucleic acid contained in the vector is replicated when the host cell divides, thereby amplifying the nucleic acids.

As used herein, the term “nucleic acid” or “polynucleotide” means any RNA or DNA, which may be unmodified or modified RNA or DNA. Polynucleotides include, without limitation, single- and double-stranded DNA, DNA that is a mixture of single- and double-stranded regions, single- and double-stranded RNA, RNA that is mixture of single-and double-stranded regions, and hybrid molecules comprising DNA and RNA that may be single-stranded or, more typically, double-stranded or a mixture of single- and double-stranded regions. In addition, polynucleotide refers to triple-stranded regions comprising RNA or DNA or both RNA and DNA. The term polynucleotide also includes DNAs or RNAs containing one or more modified bases and DNAs or RNAs with backbones modified for stability or for other reasons.

As used herein, “operably linked” with reference to nucleic acid sequences, regions, elements or domains means that the nucleic acid regions are functionally related to each other. For example, a nucleic acid encoding a leader peptide can be operably linked to a nucleic acid encoding a polypeptide, whereby the nucleic acids can be transcribed and translated to express a functional fusion protein, wherein the leader peptide affects secretion of the fusion polypeptide. In some instances, the nucleic acid encoding a first polypeptide (e.g., a leader peptide) is operably linked to nucleic acid encoding a second polypeptide and the nucleic acids are transcribed as a single mRNA transcript, but translation of the mRNA transcript can result in one of two polypeptides being expressed. For example, an amber stop codon can be located between the nucleic acid encoding the first polypeptide and the nucleic acid encoding the second polypeptide, such that, when introduced into a partial amber suppressor cell, the resulting single mRNA transcript can be translated to produce either a fusion protein containing the first and second polypeptides, or can be translated to produce only the first polypeptide. In another example, expression control sequences can be operably linked to nucleic acid encoding a polypeptide of interest to initiate, regulate or otherwise control transcription of the nucleic acid encoding the polypeptide of interest.

As used herein, the term “pharmaceutically-acceptable carrier” is intended to include any and all solvents, dispersion media, coatings, antibacterial and antifungal compounds, isotonic and absorption delaying compounds, and the like, compatible with pharmaceutical administration. Pharmaceutically-acceptable carriers and their formulations are known to one skilled in the art and are described, for example, in Remington's Pharmaceutical Sciences (20th edition, ed. A. Gennaro, 2000, Lippincott, Williams & Wilkins, Philadelphia, PA.).

As used herein, the terms “polypeptide,” “peptide” and “protein” are used interchangeably herein to mean a polymer comprising two or more amino acids joined to each other by peptide bonds or modified peptide bonds, i.e., peptide isosteres. Polypeptide refers to both short chains, commonly referred to as peptides, glycopeptides or oligomers, and to longer chains, generally referred to as proteins. Polypeptides may contain amino acids other than the 20 gene-encoded amino acids. Polypeptides include amino acid sequences modified either by natural processes, such as post-translational processing, or by chemical modification techniques that are well known in the art. Such modifications are well described in basic texts and in more detailed monographs, as well as in a voluminous research literature.

As used herein, “prevention” or “preventing” of a disorder or condition refers to a compound that, in a statistical sample, reduces the occurrence of the disorder or condition in the treated sample relative to an untreated control sample, or delays the onset of one or more symptoms of the disorder or condition relative to the untreated control sample.

As used herein, the term “pseudotyping” refers to the generation of viral vectors that carry foreign viral envelope proteins on their surface. Viral surface glycoproteins modulate viral entry into the host cell by interacting with particular cellular receptors to induce membrane fusion. For instance, the native HIV envelope glycoprotein specifically binds the receptor CD4 as well as co-receptors CXCR4 or CCR5, which effectively limits the potential target cells of HIV to CD4⁺ T-cells and monocytes. To change or broaden the selection of cells that may be transduced, lentiviral vectors are therefore usually pseudotyped with heterologous envelope glycoproteins both from related and unrelated viruses. Dependent on the choice of glycoprotein used for pseudotyping, the tropism of the virus, i.e., the range of host cells that can be infected by the lentivirus, can thus be either expanded or directed to particular cell types of interest.

As used herein, the term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the material is derived from a cell so modified. Thus, for example, recombinant cells express genes that are not found within the native (non-recombinant) form of the cell or express native genes that are otherwise abnormally expressed, under expressed or not expressed at all.

As used herein, an endogenous nucleic acid sequence in the genome of an organism (or the encoded protein product of that sequence) is deemed “recombinant” herein if a heterologous sequence is placed adjacent to the endogenous nucleic acid sequence, such that the expression of this endogenous nucleic acid sequence is altered. In this context, a heterologous sequence is a sequence that is not naturally adjacent to the endogenous nucleic acid sequence, whether or not the heterologous sequence is itself endogenous to the organism (originating from the same organism or progeny thereof) or exogenous (originating from a different organism or progeny thereof). By way of example, a promoter sequence can be substituted (e.g., by homologous recombination) for the native promoter of a gene in the genome of an organism, such that this gene has an altered expression pattern. This gene would be “recombinant” because it is separated from at least some of the sequences that naturally flank it. A nucleic acid is also considered “recombinant” if it contains any modifications that do not naturally occur in the corresponding nucleic acid in a genome. For instance, an endogenous coding sequence is considered “recombinant” if it contains an insertion, deletion or a point mutation introduced artificially, e.g., by human intervention. A “recombinant nucleic acid” also includes a nucleic acid integrated into a host cell chromosome at a heterologous site and a nucleic acid construct present as an episome.

As used herein, the term “separate” therapeutic use refers to an administration of at least two active ingredients at the same time or at substantially the same time by different routes.

As used herein, the term “sequential” therapeutic use refers to administration of at least two active ingredients at different times, the administration route being identical or different. More particularly, sequential use refers to the whole administration of one of the active ingredients before administration of the other or others commences. It is thus possible to administer one of the active ingredients over several minutes, hours, or days before administering the other active ingredient or ingredients. There is no simultaneous treatment in this case.

As used herein, the term “simultaneous” therapeutic use refers to the administration of at least two active ingredients by the same route and at the same time or at substantially the same time.

As used herein, the terms “subject,” “individual,” or “patient” are used interchangeably and refer to an individual organism, a vertebrate, a mammal, or a human. In certain embodiments, the individual, patient or subject is a human.

“Treating”, “treat”, or “treatment” as used herein covers the treatment of a disease or disorder described herein, in a subject, such as a human, and includes: (i) inhibiting a disease or disorder, i.e., arresting its development; (ii) relieving a disease or disorder, i.e., causing regression of the disorder; (iii) slowing progression of the disorder; and/or (iv) inhibiting, relieving, or slowing progression of one or more symptoms of the disease or disorder. In some embodiments, treatment means that the symptoms associated with the disease are, e.g., alleviated, reduced, cured, or placed in a state of remission.

It is also to be appreciated that the various modes of treatment of medical diseases and conditions as described herein are intended to mean “substantial,” which includes total but also less than total treatment, and wherein some biologically or medically relevant result is achieved. The treatment may be a continuous prolonged treatment for a chronic disease or a single, or few time administrations for the treatment of an acute condition.

As used herein, a “vector” is a replicable nucleic acid from which one or more heterologous proteins can be expressed when the vector is transformed into an appropriate host cell. Reference to a vector includes those vectors into which a nucleic acid encoding a polypeptide or fragment thereof can be introduced, typically by restriction digest and ligation. Reference to a vector also includes those vectors that contain nucleic acid encoding a polypeptide. The vector is used to introduce the nucleic acid encoding the polypeptide into the host cell for amplification of the nucleic acid or for expression/display of the polypeptide encoded by the nucleic acid. The vectors typically remain episomal, but can be designed to effect integration of a gene or portion thereof into a chromosome of the genome. Also contemplated are vectors that are artificial chromosomes, such as yeast artificial chromosomes and mammalian artificial chromosomes. Selection and use of such vehicles are well known to those of skill in the art.

As used herein, a vector also includes “virus vectors” or “viral vectors.” Viral vectors are engineered viruses that are operatively linked to exogenous genes to transfer (as vehicles or shuttles) the exogenous genes into cells.

As used herein, an “expression vector” includes vectors capable of expressing DNA that is operatively linked with regulatory sequences, such as promoter regions, that are capable of effecting expression of such DNA fragments. Such additional segments can include promoter and terminator sequences, and optionally can include one or more origins of replication, one or more selectable markers, an enhancer, a polyadenylation signal, and the like. Expression vectors are generally derived from plasmid or viral DNA, or can contain elements of both. Thus, an expression vector refers to a recombinant DNA or RNA construct, such as a plasmid, a phage, recombinant virus or other vector that, upon introduction into an appropriate host cell, results in expression of the cloned DNA. Appropriate expression vectors are well known to those of skill in the art and include those that are replicable in eukaryotic cells and/or prokaryotic cells and those that remain episomal or those which integrate into the host cell genome.

As used herein, “third generation of lentiviral vector systems” refer to systems that employ so-called self-inactivating (SIN) lentiviral vectors. When lentiviral vectors are integrated into a host cell genome, the transgene cassette of the provirus comprising the gene of interest is flanked by two long terminal repeats (LTRs). The presence of these LTRs may promote the emergence of potentially harmful replication-competent recombinants. In addition, viral promoter/enhancer regions located in the LTRs could induce the expression of adjacent host genes, with potentially tumorigenic consequences. Moreover, the promoter/enhancer regions in these LTRs can transcriptionally interfere with the promoter driving the transgene as well as the neighboring genes. Therefore, promoter/enhancer sequences of the viral 3′ LTR were removed by deleting a particular region inside the LTR (U3) from the DNA that was used to produce the viral RNA. Deletion of the U3 region effectively abolished the transcriptional activity of the LTR. Furthermore, tat, a regulatory gene driving viral transcription, was deleted from the packaging plasmid, whereas the second regulatory gene rev was provided from another separate, fourth, plasmid (Zufferey et al., 1998, Self-Inactivating Lentivirus Vector for Safe and Efficient in Vivo Gene Delivery. J Virol 72 (12), 9873-9880; Schambach et al., 2013, Biosafety Features of Lentiviral Vectors. Human Gene Therapy 24, 132-142). Accordingly, the lentiviral vector of the present technology is replication incompetent due to the split packaging design and self-inactivating (SIN) due to a deletion in the U3 region of the 3′ LTR.

### Usher Disease

Usher disease is a diverse group of genetic disorders that result in impairment of inner ear function and vision and that can be broadly classified into three types. Usher type 1 patients present with congenital prelingual hearing loss and develop variable degrees of bilateral vestibular hypofunction. Subsequent development of retinitis pigmentosa leads to blindness. Usher type 1 is due to defects in myosin VIIA, harmonin, cadhedrin-23, protocadhedrin-15, sans or CIB2. Usher type 1 is generally the most severe form of the disease and has an incidence of 1/25,000. The hearing manifestations of the disease are currently treated with cochlear implantation. The combination of vestibular hypofunction with visual impairment leads to severe imbalance, which is currently not treatable.

Among Usher type I patients, 53-63% of individuals are classified as subtype B (USH1B), which is caused by a variety of mutations in the myosin VIIA gene. The 6645 bp coding sequence of MYO7A produces a 2215 amino acid atypical myosin that plays a key role in mechanosensory transduction in the inner ear. Myosin VIIA mutations cause a spectrum of developmental and progressive disorders of the stereocilia on cochlear and/or vestibular hair cells, ranging from USH1B (recessive inheritance) and atypical Usher syndrome to recessive and dominant forms of hearing loss only (DFNB2 and DFNA11), depending on the location of the mutation. USH1B also has a variable presentation and phenotype. Mutations in myosin VIIA have been shown to cause the phenotype in the Shaker-1 mouse, which was first described in 1924. Despite the initial presence of hearing, thresholds are lost between approximately 18 and 21 days of age. Interestingly, at that time there still is a normal appearing complement of hair cells and spiral ganglion neurons, which degenerate by 3 months of age. In contrast to USH1B patients, vision is not affected in the mice.

Several studies on mouse models of other types of Usher syndrome have suggested that rescue of the genetic defect is most effective prior to loss of function, or that a functional copy of the defective gene has to be delivered before maturation of hearing. Although hearing loss has a congenital onset, studies of USH1B patients demonstrate commence of severe to profound loss of residual hearing even into adulthood. Importantly, in contrast to previous studies, in-vivo vector administration yielded reporter gene expression in both inner and outer hair cells as well as in spiral ganglion neurons.

### Lentiviral Systems

Like that of other retroviruses, the genome of lentiviruses consists of a single-stranded (ss) positive sense RNA. During replication, the lentiviral ssRNA genome is converted into double-stranded (ds) DNA by a process known as reverse transcription. The reverse transcribed lentiviral dsDNA is subsequently integrated into the host cell's genome, which in turn replicates and transcribes the integrated lentiviral genes along with its own genes to produce new viral particles. The lentiviral, e.g., HIV-1, genome comprises three major structural genes: gag, pol and env. gag encodes the viral matrix (MA), capsid (CA) and nucleocapsid (NC), which collectively facilitate the assembly and release of the virus particles. The pol gene encodes the viral enzymes protease (PR), reverse transcriptase (RT) and integrase (IN), which govern viral replication. The HIV-1 env encodes the viral surface glycoprotein gp160, which is subsequently cleaved to form the surface protein gp120 and the transmembrane protein gp41 during viral maturation. In addition to the structural genes gag, pol and env, the HIV-1 genome furthermore comprises the two regulatory genes tat and rev as well as the four accessory genes vif, vpr, vpu and nef. Whereas tat encodes a transactivator required for viral transcription, re encodes a protein that controls both splicing and export of viral transcripts. The four accessory genes are considered non-essential for viral replication, but are believed to increase its efficiency (German Advisory Committee Blood (Arbeitskreis Blut), Subgroup ‘Assessment of Pathogens Transmissible by Blood’, 2016, Human Immunodeficiency Virus (HIV). Transfus Med Hemother. 43 (3), 203-222).

To meet biosafety concerns, the first generation of lentiviral vector systems split the viral genome into three separate plasmids to avoid the formation of replication-competent viruses. The first plasmid encoded the actual vector that was to be integrated into the host cell's genome. It comprised the transgene of interest functionally linked to a suitable promoter sequence as well as cis-elements necessary for polyadenylation, integration, initiation of reverse transcription and packaging (e.g., the long-terminal repeats, the packaging signal, the primer binding site, the polypurine tract or the Rev-responsive element). The second plasmid, known as packaging plasmid, comprised the genes encoding the viral proteins that contribute to packaging, reverse transcription and integration of the viral genome, i.e., gag, pol, the regulatory genes tat and rev as well as the four accessory genes vif, vpu, vpr and nef. The third plasmid (Env plasmid) expressed the viral glycoprotein for host cell receptor binding. Due to the physical separation of the packaging genes from the rest of the viral genome, this split genome design prevented viral replication after infection of the host cell.

To further improve the safety of lentiviral vectors, a second generation system was established by removing all accessory genes from the packaging plasmid, as they were found to constitute crucial virulence factors.

With the third generation of lentiviral vector systems, so-called self-inactivating (SIN) vectors were introduced. When lentiviral vectors are integrated into a host cell genome, the transgene cassette of the provirus comprising the gene of interest is flanked by two long terminal repeats (LTRs). The presence of these LTRs may promote the emergence of potentially harmful replication-competent recombinants. In addition, viral promoter/enhancer regions located in the LTRs could induce the expression of adjacent host genes, with potentially tumorigenic consequences. Moreover, the promoter/enhancer regions in these LTRs can transcriptionally interfere with the promoter driving the transgene as well as the neighboring genes. Therefore, promoter/enhancer sequences of the viral 3′ LTR were removed by deleting a particular region inside the LTR (U3) from the DNA that was used to produce the viral RNA. Deletion of the U3 region effectively abolished the transcriptional activity of the LTR. Furthermore, tat, a regulatory gene driving viral transcription, was deleted from the packaging plasmid, whereas the second regulatory gene rev was provided from another separate, fourth, plasmid (Zufferey et al., 1998, Self-Inactivating Lentivirus Vector for Safe and Efficient in Vivo Gene Delivery. J Virol 72 (12), 9873-9880; Schambach et al., 2013, Biosafety Features of Lentiviral Vectors. Human Gene Therapy 24, 132-142).

### Methods of the Present Technology

The present technology provides a composition for use in treating or preventing Usher 1B syndrome in a subject, wherein the composition comprises a 3^{rd} generation lentiviral vector pseudotyped with a viral envelope glycoprotein capable of binding to a receptor expressed in a cell of the inner ear, wherein said composition has a titer of at least 7.78×10⁶ TU/mL and is administered to the inner ear of the subject.

The inner ear refers to the innermost part of the vertebrate ear and is responsible for sound detection and balance. The mammalian inner ear consists of the bony labyrinth comprising the cochlea, which is required for hearing, and the vestibular system that is dedicated to balance. The cochlea is a spiral or snail shaped structure containing three fluid filled compartments or scalae. The scala vestibuli (vestibular duct) is filled with Na⁺-rich and K⁺-low perilymph and terminates at the oval window. The scala tympani (tympanic duct) is also filled with perilymph and abuts the round window. Located between the scala vestibule and the scala tympani is the scala media (cochlear duct). It contains endolymph, which is low in Nat and high in K⁺. The stria vascularis, which forms the outer wall of the scala media, pumps Na⁺ and K⁺ against their concentration gradients to create an electrical potential between endo- and perilymph known as endocochlear potential. The scala media furthermore contains the organ of Corti that sits on top of the basilar membrane, which separates the scala media from the scala tympani. The organ of Corti consists of mechanosensory epithelial cells known as inner and outer hair cells as well as the rods of Corti and a variety of supporting cells (Morgan et al., 2020, Gene therapy as a possible option to treat hereditary hearing loss. Medizinische Genetik).

When sound waves reach the outer ear, air pressure pushes against the eardrum (tympanic membrane) and induces mechanical movement of the three ossicles malleus, incus and stapes, which in turn transmit vibrations to the oval window. The pressure applied to the oval membrane triggers movement of the perilymph and endolymph within the cochlea. Motion of these inner ear fluids results in the bending of the basilar membrane and causes a plurality of stereocilia on top of the hair cells to deflect. In consequence, mechanically gated K⁺ channels open to allow small positive ions to enter, thereby causing depolarization of the hair cells. The hair cells subsequently release neurotransmitters that bind to receptors of spiral ganglion neurons (SGN). The SGN in turn fire action potentials to the brain via the cochlear nerve (Morgan et al., 2020, Gene therapy as a possible option to treat hereditary hearing loss. Medizinische Genetik).

In one aspect, the present disclosure provides a method for treating or preventing Usher 1B syndrome in a patient in need thereof comprising administering to the patient an effective amount of a composition comprising a self-inactivating (SIN) lentiviral expression vector pseudotyped with a viral envelope glycoprotein that is configured to bind to a receptor expressed in an inner ear cell, wherein the SIN lentiviral expression vector comprises: a 5′ long terminal repeat (LTR) region comprising or consisting of a constitutively active or inducible heterologous promoter sequence and the Repeat (R)-U5 sequence of SEQ ID NO: 4, wherein the constitutively active heterologous promoter sequence is located upstream of the R-U5 sequence; a 5′ untranslated region (UTR) comprising the splice donor (SD) sequence of SEQ ID NO: 5, a packaging signal sequence, a Rev-responsive element (RRE), the splice acceptor (SA) sequence of SEQ ID NO: 9, and optionally, a polypurine tract (PPT) region; an internal promoter operably linked to a cargo sequence, wherein the cargo sequence comprises a gene sequence encoding MyoVIIa isoform 1, and optionally a gene sequence encoding a reporter protein; RNA processing elements comprising a posttranscriptional regulatory element (PRE), wherein the PRE is located downstream of the cargo sequence, and a 3′ LTR region comprising or consisting of the deleted U3-R sequence of SEQ ID NO: 11, wherein the 3′ LTR region is located downstream of the PRE, and wherein the SIN lentiviral expression vector does not comprise a PPT region between the cargo sequence and the PRE. In some embodiments, the 5′ UTR further comprises a primer binding site sequence, optionally wherein the primer binding site sequence comprises SEQ ID NO: 13. Additionally or alternatively, in some embodiments, the gene sequence encoding MyoVIIa isoform 1 comprises SEQ ID NO: 1. In any of the preceding embodiments, the PRE comprises SEQ ID NO: 10. The SIN lentiviral expression vector may be derived from a lentivirus selected from the group consisting of human immunodeficiency virus (e.g., HIV-1, HIV-2), simian immunodeficiency virus (SIV), feline immunodeficiency virus (FIV), and equine infectious anemia virus (EIAV). Additionally or alternatively, the SIN lentiviral expression vector lacks vif, vpr, vpu, nef, and optionally tat genes and/or wherein the SIN lentiviral expression vector comprises a PPT region of SEQ ID NO: 41 between the PRE and the 3′ LTR region.

Additionally or alternatively, in some embodiments, the packaging signal sequence comprises SEQ ID NO: 6 and/or the RRE comprises SEQ ID NO: 7. In some embodiments, the PPT region of the 5′ UTR comprises SEQ ID NO: 8 and/or is located downstream of the RRE.

In some embodiments, the constitutively active heterologous promoter sequence is a Rous sarcoma virus (RSV) promoter sequence, a human cytomegalovirus (CMV) promoter sequence, an HIV promoter sequence, a eukaryotic promoter sequence (e.g., EF1a, PGK1, UBC, or human beta actin). In certain embodiments, the RSV promoter sequence comprises SEQ ID NO: 3. In other embodiments, the inducible heterologous promoter sequence is a tetracycline response element (TRE) that can be bound by a tetracycline transactivator (tTA) protein in the presence of tetracycline or an analogue thereof, e.g. doxycycline.

The internal promoter may be a promoter selected from the group comprising a viral promoter, a cellular promoter, a cell-specific promoter, an inducible promoter or a synthetic promoter. It may, e.g., be a viral promoter selected from the group comprising a promoter derived from CMV, spleen focus-forming virus (SFFV), myeloproliferative sarcoma virus (MPSV), murine embryonal stem cell virus (MESV), murine leukemia virus (MLV) and simian virus 40 (SV40). In some embodiments, the internal promoter is a promoter derived from CMV or SFFV. In another embodiment, the promoter may also be a CAG promoter, i.e. a composite construct consisting of the CMV enhancer fused to the chicken beta-actin promoter and the rabbit beta-Globin splice acceptor site. Additionally or alternatively, in certain embodiments, the SFFV promoter sequence comprises SEQ ID NO: 2. In other embodiments, the CAG promoter comprises SEQ ID NO: 18.

The internal promoter may alternatively be a cellular promoter selected from the group comprising EF1a, PGK1 and UBC. It may also be a promoter that enables expression of the cargo sequence in a cell-specific manner. It may, e.g., be a hair cell-specific promoter selected from the group comprising POU4F3, POU3F4, ATOHI, Prestin, Pendrin and MY07A or an SGN-specific promoter selected from the group comprising e.g. MAP1 B, SYN, and NSE.

The internal promoter may also be an inducible promoter, especially, if expression of the cargo sequence may lead to cytotoxicity. The promoter may thus be selected from the group comprising a TET-inducible promoter, a Cumate-inducible promoter, an electrosensitive promoter and an optogenetic switch. Electrosensitive promoters or optogenetic switches may be especially useful when expression of the cargo sequence is to be controlled by a cochlea implant.

The internal promoter region may also be a hybrid or a synthetic promoter selected from the group comprising CAG and MND. A hybrid promoter is composed of several regulatory elements from different promoters/enhancers, which are joined to build a new promoter combining the desired features. In contrast, a synthetic promoter essentially consists of an array of transcription factor binding sites fused to a minimal promoter.

In other embodiments, the internal promoter region may comprise a chromatin opening element, i.e., a DNA sequence consisting of methylation-free CpG islands that either encompasses a housekeeping gene promoter or is operably linked to a heterologous promoter to confer reproducible, stable transgene expression.

The cargo sequence may be a complementary DNA (cDNA) that has been generated via reverse transcription from messenger RNA (mRNA) transcribed from MyoVIIa isoform 1 (and optionally the reporter protein) and that lacks introns normally present in MyoVIIa isoform 1 (and optionally the reporter protein). Alternatively, it may be the complete gene of interest comprising introns and, optionally, regulatory regions provided that the length of the cargo sequence does not exceed the packaging limit of the lentiviral vector. The cargo sequence may alternatively be synthetically engineered, it may, e.g., be codon-optimized to ensure efficient expression in the subject, e.g., codon-optimized for expression in human cells. The cargo sequence may, optionally, further comprise an internal ribosomal entry site (IRES), i.e. an RNA element that mediates mRNA translation in the absence of a 5′ cap and allows for co-expression of multiple genes under the control of a single promoter. In some embodiments, an IRES is interspersed between the gene sequence encoding MyoVIIa isoform 1, and the gene sequence encoding a reporter protein, optionally wherein the IRES comprises SEQ ID NO: 15. Examples of reporter proteins include, but are not limited to dTomato, TagBFP, Azurite, EBFP2, mKalama1, Sirius, Sapphire, T-Sapphire, ECFP, Cerulean, SCFP3A, mTurquoise, monomeric Midoriishi-Cyan, TagCFP, mTFP1, EGFP, Emerald, Superfolder GFP, Monomeric Azami Green, TagGFP2, mUKG, mWasabi, EYFP, Citrine, Venus, SYFP2, TagYFP, Monomeric Kusabira-Orange, mKOK, mKO2, mOrange, mOrange2, mRaspberry, mCherry, dsRed, mStrawberry, mTangerine, tdTomato, TagRFP, TagRFP-T, mApple, mRuby, mPlum, HcRed-Tandem, mKate2, mNeptune, NirFP, TagRFP657, IFP1.4, iRFP, mKeima Red, LSS-mKatel, LSS-mKate2, PA-GFP, PAmCherry 1, PATagRFP, Kaede (green), Kaede (red), KikGR1 (green), KikGR1 (red), PS-CFP2, PS-CFP2, mEos2 (green), mEos2 (red), PSmOrange, and Dronpa

Additionally or alternatively, the 5′ UTR comprises a mutant group-specific antigen (Gag) sequence. In one embodiment, the mutant Gag sequence comprises SEQ ID NO: 14.

In some embodiments, the SIN lentiviral vector is pseudotyped with a viral envelope glycoprotein capable of binding to a receptor selected from the group consisting of the LDL-receptor and LDL-R family members, the SLC1A5-receptor, the Pit1/2-receptor and the PIRYV-G-receptor. The viral envelope glycoprotein capable of binding the LDL-receptor or LDL-R family members may be, e.g., MARAV-G, COCV-G, VSV-G or VSV-G ts. In other embodiments, the SIN lentiviral vector is pseudotyped with a viral envelope glycoprotein that is capable of binding the SLC1A5-receptor. Such viral envelope glycoproteins may be derived from RD114 glycoprotein (GP), or BaEV GP. In certain embodiments, the SIN lentiviral vector is pseudotyped with a viral envelope glycoprotein that is capable of binding the Pit1/2-receptor such as gibbon ape leukemia virus (GALV) GP, the amphotropic murine leukemia virus (A-MuLV) GP or 10A1 MLV GP. The viral envelope glycoprotein capable of binding the PIRYV-G-receptor may be, e.g., PIRYV-G.

In some embodiments, the SIN lentiviral vector of the present technology is pseudotyped with a viral envelope glycoprotein comprising an amino acid sequence having at least 80%, at least 85%, at least 90%, at least 95%, at least 96%, at least 97%, at least 98%, at least 99% or 100% sequence identity to any one of SEQ ID NOs: 20, 22, 24, 26, 28, 30, 32, 34, 36, 38, or 40. In certain embodiments, the SIN lentiviral vector of the present technology is pseudotyped with a viral envelope glycoprotein encoded by a nucleic acid sequence having at least 80%, at least 85%, at least 90%, at least 95%, at least 96%, at least 97%, at least 98%, at least 99% or 100% sequence identity to any one of SEQ ID NOs: 19, 21, 23, 25, 27, 29, 31, 33, 35, 37, or 39.

Optionally, the lentiviral vector may be pseudotyped with more than one type of heterologous glycoprotein. It may, e.g., be pseudotyped with two different glycoproteins targeting the same receptor protein, such as VSV-G and COCV-G. Alternatively, the lentiviral vector may be pseudotyped with a mixture of glycoproteins that bind to at least two, i.e. two, three or four different receptor proteins. It may, e.g., be simultaneously pseudotyped with COCV-G binding to the LDL receptor and RD114 binding to SLC1 A5.

Additionally or alternatively, in some embodiments, the methods of the present technology comprise administering to the patient a second vector comprising gag and pol, and optionally tat, a third plasmid comprising the viral envelope glycoprotein, and a fourth vector comprising regulatory gene Rev. Lentiviral vectors can be generated by co-expressing the different viral constituents in a single packaging cell. At least four distinct expression plasmids may be cotransfected into a packaging cell to produce the lentiviral vector at sufficiently high titer. The packaging cell may be selected from the group comprising a HEK293T cell, a HEK293 cell, a NIH3T3 cell, a HeLa cell, an HT1080 cell, a COS-1 cell and an AGE1.CR cell. The ratio of the Gag/Pol encoding packaging construct, the Env plasmid, the Rev plasmid and the vector plasmid may be 10-20:1-10:5-10:5:10, preferably 13-16:1-7:6-8:6-8. Especially the amount of Env-encoding plasmid may vary considerably depending on the selection of the glycoprotein for lentiviral vector pseudotyping. The medium comprising the produced lentiviral particles may be harvested 25-40 h, e.g. 30-36 h post-transfection of the packaging cells. Optionally, a second harvest of newly generated lentiviral particles may be performed 40-60 h, e.g. 48-54 h post-transfection. Preferably, the medium comprising the lentiviral particles is filtered using a 0.22 μm pore size filter. Media comprising virus particles obtained from subsequent harvests may be pooled and, optionally, stored, e.g., at −80 °C. Co-expression of multiple plasmids in a single packaging cell such as a HEK293T cell enables the production of lentiviral particles at high titers.

The efficiency of gene transfer and in turn of gene expression, however, largely depends on the total number of vector particles delivered per target cell (Zhang et al, 2001). The composition of the present technology, therefore, needs to possess a virus titer high enough to deliver a sufficient amount of lentiviral particles to facilitate effective transduction of inner ear cells. The viral titer of a composition is most accurately provided as functional titer, i.e. it describes how many viral particles have actually infected a target cell. It is, therefore, also designated an infectious titer. It is commonly expressed as transduction units per mL (TU/mL) and can be assessed after transduction of a cell, e.g., by PCR-based methods or flow cytometry. The final virus titer of the present composition should be of a magnitude of at least 10⁶, at least 10⁷, at least 10⁸ TU/mL. It may even be higher, e.g., at least 10° TU/mL or even at least 10¹⁰ TU/mL.

To reach a functional titer of the above mentioned magnitude, the lentiviral vector titer of the composition according to the present technology may be further concentrated after harvesting of the lentiviral particles from packaging cells, e.g., using ultracentifugation. In one embodiment, the lentiviral vector titer is concentrated using ultracentrifugation, wherein the viral vector particles are centrifuged at at least 10,000×g for at least 1 h, at least 1.5 h or, for at least 2 h, at least 2.5 h, at least 3 h, at least 3.5 h or at least 4 h (Zhang et al., 2001,). The speed and duration of centrifugation depend on the respective pseudotype of the viral vector particle. VSV-G-pseudotyped lentiviral vector particles may, for instance, be centrifuged at 82,740×g for 2 h. In contrast, vector particles pseudotyped with RD114, GALV or BaEV may be centrifuged at about 13,238×g, but for longer time periods, e.g., at least 4 h, overnight. Using ultracentrifugation, it is possible to concentrate the virus titer of the composition of the present technology by at least 10 fold, by at least 20 fold, by at least 30 fold, by at least 40 fold, by at least 50 fold, by at least 60 fold, by at least 70 fold, by at least 80 fold, by at least 90 fold, by at least 100 fold, by at least 150 fold, by at least 200 fold, by at least 250 fold, or even by at least 300 fold. However, even though ultracentrifugation has proven highly effective for harvesting lenti viral vectors, the method is associated with certain draw-backs: The rotors that may be used at such speed usually have only a small volume capacity and the attainment of large volumes of high-titer viral vectors thus is very time-consuming (Zhang et al., 2001). Ultracentrifugation further requires the virus particles to exhibit sufficient stability, e.g., due to pseudotyping of the viral envelope. Therefore, in another embodiment, the virus titer may also be concentrated by any other suitable purification method known from the state of the art, such as microfiltration, ultrafiltration, chromatography, density gradient ultracentrifugation, tangential flow microfiltration, or precipitation.

After concentrating the lentiviral particles of the present technology by performing ultracentrifugation or any other suitable method from the state of the art, the viral particles have to be reconstituted in a suitable medium. The medium should have a physiological salt concentration and a physiological pH. The viral particles may be re-suspended in a medium comprising PBS (pH 7.2-7.4) and 1-50 mM, e.g., 1-10 mM, 20-30 mM, 30-40 mM, 40-50 mM, or 10-20 mM HEPES. In some embodiments, the medium used for reconstituting the concentrated viral particles does not contain any serum, hormones or cytokines to avoid any external biological stimulus on inner ear cells. Optionally, the medium may also be serum or blood. It may also be a lymphatic liquid of the inner ear, e.g., perilymph or endolymph.

The composition of the present technology may be stored for up 1-4 weeks, e.g., 1 week, 2 weeks, 3 weeks or 4 weeks at temperatures around 4 °C. The composition may be stored in a frozen state, e.g., at about-80 °C. When frozen, the composition may be stored for at least 6, at least 12, at least 24, or at least 36 months prior to being administered to the subject.

The cell of the inner ear that is to be transduced by the lentiviral vector according to the present technology may be a cell selected from the group consisting of cells of the organ of Corti, including inner and outer hair cells, spiral ganglion neurons and glial cells, cells of the stria vascularis and spiral ligament and lateral wall fibrocytes. In some embodiments, the cell is a hair cell or a spiral ganglion neuron. Hair cells constitute the sensory cells required for hearing and are located within the organ of Corti. They are arranged into one row of inner hair cells followed by three rows of outer hair cells. Hair cells possess apical modifications, so-called stereocilia, which are in contact with the fluid filling the scala media, the endolymph, and interdigitate with a variety of supporting cells. Unlike, e.g., birds or reptiles, mammals are incapable of naturally regenerating damaged or lost hair cells. Accordingly, hair cell degeneration may lead to permanent hearing loss if left untreated.

Spiral ganglion neurons (SGNs) are located within the modiolus, i.e., the central axis of the cochlea. Their dendrites form synapses with the base of hair cells and their axons run into the eighth cranial nerve, also known as the vestibulocochlear nerve. SGNs are the first neurons in the auditory systems that fire action potentials upon perception of sound. Glial cells are non-neuronal cells that do not produce electrical impulses and act as supporting cells of the nervous system. Glial cells have the potential to become reprogrammed into primary auditory neurons (PANs) through artificial expression of certain transcription factors (Meas et al., 2018, Reprogramming Glia Into Neurons in the Peripheral Auditory System as a Solution for Sensorineural Hearing Loss: Lessons From the Central Nervous System. Front Mol Neurosci. 11:77). Glial cells thus represent an attractive target for lentiviral gene transfer to compensate for the loss of neurons in the peripheral auditory system. The stria vascularis consists of epithelial cells and forms the outer wall of the scala media. It furthermore produces the endolymph of the scala media. The spiral ligament secures the membranous scalar media to the spiral canal of the cochlea.

The inner ear cell transduced according to the present technology may alternatively be type I and II vestibular hair cells, vestibular supporting cells and vestibular ganglion neurons. The vestibular system is the sensory system of the inner ear that facilitates the sense of balance and spatial orientation for coordinated movement. Accordingly, the composition according to the present technology may also be suitable to alleviate symptoms associated with vestibular disturbances.

In another embodiment, the cell of the inner ear may also be a supporting cell selected from the group comprising a Boettcher cell, a Claudius cell, a Deiters' cell, a Hensen's cell or a pillar cell. Boettcher cells are located on the basilar membrane in the lower turn of the cochlea where they project microvilli into the intercellular space and may perform both secretory and absorptive functions. Claudius cells are located immediately above the Boettcher cells and are in direct contact with the endolymph. The formation of tight junctions between adjacent Claudius cells prevents the leakage of endolymph out of the scala media. Deiters' cells are arranged in up to 5 rows and sit directly on the basilar membrane of the cochlea. They form long, apical cell extensions that extend to the reticular lamina of the inner ear. The tall Hensen's cells are located adjacent to the outer row of Deiters' cells within the organ of Corti where they may mediate ion metabolism. Hensen's cells are a particularly interesting target for gene therapy as they are believed to have retained a capacity to regenerate hair cells (Malgrange et al., 2002, Epithelial supporting cells can differentiate into outer hair cells and Deiters' cells in the cultured organ of Corti. Cellular and Molecular Life Sciences. 59, 1744-1757). For example, a cargo nucleic acid may stimulate the regeneration of hair cells by Hensen's cells. Pillar cells can be divided in outer and inner pillar cells. Both types form numerous cross-linked microtubules and actin filaments. Pillar cells obtained their name because the outer cells are free standing and form contacts to adjacent cells only at their bases and apices.

The composition of the present technology may be administered to the subject by any method suitable for delivering a substance to cells of the inner ear. The method for delivering the composition to a subject may, for instance, be a method selected from the group comprising a cochlea implant route, round window injection, oval window injection, canalostomy, cochleostomy as well as injection into the endolymphatic sac. For round window injection, a long needle is inserted through the outer ear and punctures the eardrum to inject the composition of the present technology through the round window located in the medial wall of the middle ear into the scala tympani. Oval window injection involves injection of the composition through the oval window into the scala vestibuli. Alternatively, the composition may be injected into the endolymphatic sac, a small pouch connected via the endolymphatic duct to the endolymph-containing scala media and, thus, the compartment harboring the organ of Corti. However, injection into the endolymphatic sac requires a mastoidectomy that is associated with a large decompression of the posterior fossa plate. Thus, there is a risk associated with injection into the endolymphatic sac (Blanc et al., 2020). Cochleostomy is a method where the viral vector is injected directly into the cochlea. As previously described, direct injection into the cochlea harbors the risk of mechanically damaging sensory hair cells, leading to a decline in hearing function. During canalostomy, a hole is drilled into one of the semicircular canals of the vestibular organ and the composition is injected directly into the vestibular labyrinth. Mechanical damage to the cells of the inner ear, e.g., sensory hair cells, should be avoided in the context of the present technology, especially in treatment of a human patient. Thus, the amount of the composition injected, as well as the pressure associated with the injection, should be minimized. Thus, a high titer and a high efficiency, which can be reached according to the present technology, are of particular importance for treatment of humans.

The composition of the present technology may be administered via canalostomy, in particular by injecting it into the posterior semicircular canal. Because a canalostomy does not disturb the middle ear during surgery and prevents injury of hair cells, it may also be suitable for vector delivery into the human inner ear (Blanc et al., 2020). Nevertheless, the method is associated with the risk of opening the membranous labyrinth and of a postoperative obstruction of the canal by fibrotic tissue. Improvements in the surgical techniques used, e.g., via the help of robotic assistance, may greatly reduce some of the risks associated with this method (Blanc et al., 2020). The surgical procedure conducted for canalostomy resembles that of a cochlear implant surgery (Blanc et al., 2020).

A cochlea implant is a device that is surgically introduced into the inner ear. Conventional cochlear implants consist of an external and an internal part. The external part is comprised of a microphone, a sound processor and a battery. The internal, transplanted part directs the signals received by the external part via an array of electrodes into the cochlea to directly stimulate the SGNs, thus circumventing the need for sensory hair cells to perceive sound. The implant may further be equipped with a reservoir chamber comprised of the composition of the present technology as well as drug delivery channels that transport the composition from the reservoir chamber to the tips of the electrodes for targeted delivery into the cochlea. The implant may optionally be designed to allow for sustained release of the composition. Several types of cochlea implants and other auditory prostheses capable of delivering drugs to the inner ear have been developed and may be suitable for administering the composition of the present technology to a subject (Hendricks et al., 2008, Localized Cell and Drug Delivery for Auditory Prostheses. Hear Res. 242 (1-2), 117-131). In some embodiments, when the subject is a human, the composition is administered using a cochlear implant. In a further embodiment, the cochlear implant may also deliver a growth factor, e.g., a neurotrophic factor such as BDNF, GDNF, NT3 or IGF that may support, e.g., transdifferentiation of cells into sensory hair cells or SGNs. The composition according to the present technology may also be administered to the subject by other suitable methods, including known pump-catheter systems or round window membrane diffusion, the latter comprising the use of, e.g., a collagenase for partial digestion of the round window membrane. The composition of the present technology may be used in administration to the subject via the systemic route. Intravenous injection of the composition under local anesthesia would constitute an atraumatic and easy alternative to the other methods discussed herein. Administration via the systemic route has already been successfully applied for delivery of AAVs into the murine inner ear (Shibata et al., 2017, Intravenous rAAV2/9 injection for murine cochlear gene delivery. Sci. Rep. 7, 9609).

In any and all embodiments of the methods disclosed herein, the patient is heterozygous or homozygous for a MYO7A genetic mutation. The patient may be diagnosed with Usher syndrome type IB. Accordingly, the SIN lentiviral composition of the present technology may be suitable to effectively improve or even eliminate presbycusis or balance dysfuntion in the patient.

In any of the preceding embodiments, the methods of the present technology further comprise separately, sequentially or simultaneously administering one or more additional therapeutic agents to the patient. The composition of the present technology may be administered to the patient daily, weekly, biweekly, every 3 weeks, every 4 weeks, monthly, or annually. Additionally or alternatively, in some embodiments, the composition is administered after onset of balance dysfuntion in the patient, for examples at least 1 week, at least 2 weeks, at least 3 weeks, at least 4 weeks, at least 1 month, at least 2 months, at least 3 months, at least 4 months, at least 5 months, or at least 6 months after onset of balance dysfuntion in the patient.

### EXAMPLES

### Example 1: Experimental Methods

### Cloning

The human myosin VIIA cDNA sequence as deposited in NCBI (NM_000260.4) was flanked by a 5′ Kozak consensus sequence and SgrAI/Agel restriction sites as well as a 3′ SalI restriction site by PCR.

human myosin VIIA cDNA (NM_000260.4)
>NM_000260.4 Homo sapiens myosin VIIA (MYO7A), transcript variant 1, mRNA
(SEQ ID NO: 1)
AGTGCTGGCTGGACAGCTGCTCTGGGCAGGAGAGAGAGGGAGAGACAAGAGACACACACAGAGAGACGGC
GAGGAAGGGAAAGACCCAGAGGGACGCCTAGAACGAGACTTGGAGCCAGACAGAGGAAGAGGGGACGTGT
GTTTGCAGACTGGCTGGGCCCGTGACCCAGCTTCCTGAGTCCTCCGTGCAGGTGGCAGCTGTACCAGGCT
GGCAGGTCACTGAGAGTGGGCAGCTGGGCCCCAGAACTGTGCCTGGCCCAGTGGGCAGCAGGAGCTCCTG
ACTTGGGACCATGGTGATTCTTCAGCAGGGGGACCATGTGTGGATGGACCTGAGATTGGGGCAGGAGTTC
GACGTGCCCATCGGGGCGGTGGTGAAGCTCTGCGACTCTGGGCAGGTCCAGGTGGTGGATGATGAAGACA
ATGAACACTGGATCTCTCCGCAGAACGCAACGCACATCAAGCCTATGCACCCCACGTCGGTCCACGGCGT
GGAGGACATGATCCGCCTGGGGGACCTCAACGAGGCGGGCATCTTGCGCAACCTGCTTATCCGCTACCGG
GACCACCTCATCTACACGTATACGGGCTCCATCCTGGTGGCTGTGAACCCCTACCAGCTGCTCTCCATCT
ACTCGCCAGAGCACATCCGCCAGTATACCAACAAGAAGATTGGGGAGATGCCCCCCCACATCTTTGCCAT
TGCTGACAACTGCTACTTCAACATGAAACGCAACAGCCGAGACCAGTGCTGCATCATCAGTGGGGAATCT
GGGGCCGGGAAGACGGAGAGCACAAAGCTGATCCTGCAGTTCCTGGCAGCCATCAGTGGGCAGCACTCGT
GGATTGAGCAGCAGGTCTTGGAGGCCACCCCCATTCTGGAAGCATTTGGGAATGCCAAGACCATCCGCAA
TGACAACTCAAGCCGTTTCGGAAAGTACATCGACATCCACTTCAACAAGCGGGGCGCCATCGAGGGCGCG
AAGATTGAGCAGTACCTGCTGGAAAAGTCACGTGTCTGTCGCCAGGCCCTGGATGAAAGGAACTACCACG
TGTTCTACTGCATGCTGGAGGGTATGAGTGAGGATCAGAAGAAGAAGCTGGGCTTGGGCCAGGCCTCTGA
CTACAACTACTTGGCCATGGGTAACTGCATAACCTGTGAGGGCCGGGTGGACAGCCAGGAGTACGCCAAC
ATCCGCTCCGCCATGAAGGTGCTCATGTTCACTGACACCGAGAACTGGGAGATCTCGAAGCTCCTGGCTG
CCATCCTGCACCTGGGCAACCTGCAGTATGAGGCACGCACATTTGAAAACCTGGATGCCTGTGAGGTTCT
CTTCTCCCCATCGCTGGCCACAGCTGCATCCCTGCTTGAGGTGAACCCCCCAGACCTGATGAGCTGCCTG
ACTAGCCGCACCCTCATCACCCGCGGGGAGACGGTGTCCACCCCACTGAGCAGGGAACAGGCACTGGACG
TGCGCGACGCCTTCGTAAAGGGGATCTACGGGCGGCTGTTCGTGTGGATTGTGGACAAGATCAACGCAGC
AATTTACAAGCCTCCCTCCCAGGATGTGAAGAACTCTCGCAGGTCCATCGGCCTCCTGGACATCTTTGGG
TTTGAGAACTTTGCTGTGAACAGCTTTGAGCAGCTCTGCATCAACTTCGCCAATGAGCACCTGCAGCAGT
TCTTTGTGCGGCACGTGTTCAAGCTGGAGCAGGAGGAATATGACCTGGAGAGCATTGACTGGCTGCACAT
CGAGTTCACTGACAACCAGGATGCCCTGGACATGATTGCCAACAAGCCCATGAACATCATCTCCCTCATC
GATGAGGAGAGCAAGTTCCCCAAGGGCACAGACACCACCATGTTACACAAGCTGAACTCCCAGCACAAGC
TCAACGCCAACTACATCCCCCCCAAGAACAACCATGAGACCCAGTTTGGCATCAACCATTTTGCAGGCAT
CGTCTACTATGAGACCCAAGGCTTCCTGGAGAAGAACCGAGACACCCTGCATGGGGACATTATCCAGCTG
GTCCACTCCTCCAGGAACAAGTTCATCAAGCAGATCTTCCAGGCCGATGTCGCCATGGGCGCCGAGACCA
GGAAGCGCTCGCCCACACTTAGCAGCCAGTTCAAGCGGTCACTGGAGCTGCTGATGCGCACGCTGGGTGC
CTGCCAGCCCTTCTTTGTGCGATGCATCAAGCCCAATGAGTTCAAGAAGCCCATGCTGTTCGACCGGCAC
CTGTGCGTGCGCCAGCTGCGGTACTCAGGAATGATGGAGACCATCCGAATCCGCCGAGCTGGCTACCCCA
TCCGCTACAGCTTCGTAGAGTTTGTGGAGCGGTACCGTGTGCTGCTGCCAGGTGTGAAGCCGGCCTACAA
GCAGGGCGACCTCCGCGGGACTTGCCAGCGCATGGCTGAGGCTGTGCTGGGCACCCACGATGACTGGCAG
ATAGGCAAAACCAAGATCTTTCTGAAGGACCACCATGACATGCTGCTGGAAGTGGAGCGGGACAAAGCCA
TCACCGACAGAGTCATCCTCCTTCAGAAAGTCATCCGGGGATTCAAAGACAGGTCTAACTTTCTGAAGCT
GAAGAACGCTGCCACACTGATCCAGAGGCACTGGCGGGGTCACAACTGTAGGAAGAACTACGGGCTGATG
CGTCTGGGCTTCCTGCGGCTGCAGGCCCTGCACCGCTCCCGGAAGCTGCACCAGCAGTACCGCCTGGCCC
GCCAGCGCATCATCCAGTTCCAGGCCCGCTGCCGCGCCTATCTGGTGCGCAAGGCCTTCCGCCACCGCCT
CTGGGCTGTGCTCACCGTGCAGGCCTATGCCCGGGGCATGATCGCCCGCAGGCTGCACCAACGCCTCAGG
GCTGAGTATCTGTGGCGCCTCGAGGCTGAGAAAATGCGGCTGGCGGAGGAAGAGAAGCTTCGGAAGGAGA
TGAGCGCCAAGAAGGCCAAGGAGGAGGCCGAGCGCAAGCATCAGGAGCGCCTGGCCCAGCTGGCTCGTGA
GGACGCTGAGCGGGAGCTGAAGGAGAAGGAGGCCGCTCGGCGGAAGAAGGAGCTCCTGGAGCAGATGGAA
AGGGCCCGCCATGAGCCTGTCAATCACTCAGACATGGTGGACAAGATGTTTGGCTTCCTGGGGACTTCAG
GTGGCCTGCCAGGCCAGGAGGGCCAGGCACCTAGTGGCTTTGAGGACCTGGAGCGAGGGCGGAGGGAGAT
GGTGGAGGAGGACCTGGATGCAGCCCTGCCCCTGCCTGACGAGGATGAGGAGGACCTCTCTGAGTATAAA
TTTGCCAAGTTCGCGGCCACCTACTTCCAGGGGACAACCACGCACTCCTACACCCGGCGGCCACTCAAAC
AGCCACTGCTCTACCATGACGACGAGGGTGACCAGCTGGCAGCCCTGGCGGTCTGGATCACCATCCTCCG
CTTCATGGGGGACCTCCCTGAGCCCAAGTACCACACAGCCATGAGTGATGGCAGTGAGAAGATCCCTGTG
ATGACCAAGATTTATGAGACCCTGGGCAAGAAGACGTACAAGAGGGAGCTGCAGGCCCTGCAGGGCGAGG
GCGAGGCCCAGCTCCCCGAGGGCCAGAAGAAGAGCAGTGTGAGGCACAAGCTGGTGCATTTGACTCTGAA
AAAGAAGTCCAAGCTCACAGAGGAGGTGACCAAGAGGCTGCATGACGGGGAGTCCACAGTGCAGGGCAAC
AGCATGCTGGAGGACCGGCCCACCTCCAACCTGGAGAAGCTGCACTTCATCATCGGCAATGGCATCCTGC
GGCCAGCACTCCGGGACGAGATCTACTGCCAGATCAGCAAGCAGCTGACCCACAACCCCTCCAAGAGCAG
CTATGCCCGGGGCTGGATTCTCGTGTCTCTCTGCGTGGGCTGTTTCGCCCCCTCCGAGAAGTTTGTCAAG
TACCTGCGGAACTTCATCCACGGGGGCCCGCCCGGCTACGCCCCGTACTGTGAGGAGCGCCTGAGAAGGA
CCTTTGTCAATGGGACACGGACACAGCCGCCCAGCTGGCTGGAGCTGCAGGCCACCAAGTCCAAGAAGCC
AATCATGTTGCCCGTGACATTCATGGATGGGACCACCAAGACCCTGCTGACGGACTCGGCAACCACGGCC
AAGGAGCTCTGCAACGCGCTGGCCGACAAGATCTCTCTCAAGGACCGGTTCGGGTTCTCCCTCTACATTG
CCCTGTTTGACAAGGTGTCCTCCCTGGGCAGCGGCAGTGACCACGTCATGGACGCCATCTCCCAGTGCGA
GCAGTACGCCAAGGAGCAGGGCGCCCAGGAGCGCAACGCCCCCTGGAGGCTCTTCTTCCGCAAAGAGGTC
TTCACGCCCTGGCACAGCCCCTCCGAGGACAACGTGGCCACCAACCTCATCTACCAGCAGGTGGTGCGAG
GAGTCAAGTTTGGGGAGTACAGGTGTGAGAAGGAGGACGACCTGGCTGAGCTGGCCTCCCAGCAGTACTT
TGTAGACTATGGCTCTGAGATGATCCTGGAGCGCCTCCTGAACCTCGTGCCCACCTACATCCCCGACCGC
GAGATCACGCCCCTGAAGACGCTGGAGAAGTGGGCCCAGCTGGCCATCGCCGCCCACAAGAAGGGGATTT
ATGCCCAGAGGAGAACTGATGCCCAGAAGGTCAAAGAGGATGTGGTCAGTTATGCCCGCTTCAAGTGGCC
CTTGCTCTTCTCCAGGTTTTATGAAGCCTACAAATTCTCAGGCCCCAGTCTCCCCAAGAACGACGTCATC
GTGGCCGTCAACTGGACGGGTGTGTACTTTGTGGATGAGCAGGAGCAGGTACTTCTGGAGCTGTCCTTCC
CAGAGATCATGGCCGTGTCCAGCAGCAGGGAGTGCCGTGTCTGGCTCTCACTGGGCTGCTCTGATCTTGG
CTGTGCTGCGCCTCACTCAGGCTGGGCAGGACTGACCCCGGCGGGGCCCTGTTCTCCGTGTTGGTCCTGC
AGGGGAGCGAAAACGACGGCCCCCAGCTTCACGCTGGCCACCATCAAGGGGGACGAATACACCTTCACCT
CCAGCAATGCTGAGGACATTCGTGACCTGGTGGTCACCTTCCTAGAGGGGCTCCGGAAGAGATCTAAGTA
TGTTGTGGCCCTGCAGGATAACCCCAACCCCGCAGGCGAGGAGTCAGGCTTCCTCAGCTTTGCCAAGGGA
GACCTCATCATCCTGGACCATGACACGGGCGAGCAGGTCATGAACTCGGGCTGGGCCAACGGCATCAATG
AGAGGACCAAGCAGCGTGGGGACTTCCCCACCGACAGTGTGTACGTCATGCCCACTGTCACCATGCCACC
GCGGGAGATTGTGGCCCTGGTCACCATGACTCCCGATCAGAGGCAGGACGTTGTCCGGCTCTTGCAGCTG
CGAACGGCGGAGCCCGAGGTGCGTGCCAAGCCCTACACGCTGGAGGAGTTTTCCTATGACTACTTCAGGC
CCCCACCCAAGCACACGCTGAGCCGTGTCATGGTGTCCAAGGCCCGAGGCAAGGACCGGCTGTGGAGCCA
CACGCGGGAACCGCTCAAGCAGGCGCTGCTCAAGAAGCTCCTGGGCAGTGAGGAGCTCTCGCAGGAGGCC
TGCCTGGCCTTCATTGCTGTGCTCAAGTACATGGGCGACTACCCGTCCAAGAGGACACGCTCCGTCAACG
AGCTCACCGACCAGATCTTTGAGGGTCCCCTGAAAGCCGAGCCCCTGAAGGACGAGGCATATGTGCAGAT
CCTGAAGCAGCTGACCGACAACCACATCAGGTACAGCGAGGAGCGGGGTTGGGAGCTGCTCTGGCTGTGC
ACGGGCCTTTTCCCACCCAGCAACATCCTCCTGCCCCACGTGCAGCGCTTCCTGCAGTCCCGAAAGCACT
GCCCACTCGCCATCGACTGCCTGCAACGGCTCCAGAAAGCCCTGAGAAACGGGTCCCGGAAGTACCCTCC
GCACCTGGTGGAGGTGGAGGCCATCCAGCACAAGACCACCCAGATTTTCCACAAAGTCTACTTCCCTGAT
GACACTGACGAGGCCTTCGAAGTGGAGTCCAGCACCAAGGCCAAGGACTTCTGCCAGAACATCGCCACCA
GGCTGCTCCTCAAGTCCTCAGAGGGATTCAGCCTCTTTGTCAAAATTGCAGACAAGGTCCTCAGCGTTCC
TGAGAATGACTTCTTCTTTGACTTTGTTCGACACTTGACAGACTGGATAAAGAAAGCTCGGCCCATCAAG
GACGGAATTGTGCCCTCACTCACCTACCAGGTGTTCTTCATGAAGAAGCTGTGGACCACCACGGTGCCAG
GGAAGGATCCCATGGCCGATTCCATCTTCCACTATTACCAGGAGTTGCCCAAGTATCTCCGAGGCTACCA
CAAGTGCACGCGGGAGGAGGTGCTGCAGCTGGGGGCGCTGATCTACAGGGTCAAGTTCGAGGAGGACAAG
TCCTACTTCCCCAGCATCCCCAAGCTGCTGCGGGAGCTGGTGCCCCAGGACCTTATCCGGCAGGTCTCAC
CTGATGACTGGAAGCGGTCCATCGTCGCCTACTTCAACAAGCACGCAGGGAAGTCCAAGGAGGAGGCCAA
GCTGGCCTTCCTGAAGCTCATCTTCAAGTGGCCCACCTTTGGCTCAGCCTTCTTCGAGGTGAAGCAAACT
ACGGAGCCAAACTTCCCTGAGATCCTCCTAATTGCCATCAACAAGTATGGGGTCAGCCTCATCGATCCCA
AAACGAAGGATATCCTCACCACTCATCCCTTCACCAAGATCTCCAACTGGAGCAGCGGCAACACCTACTT
CCACATCACCATTGGGAACTTGGTGCGCGGGAGCAAACTGCTCTGCGAGACGTCACTGGGCTACAAGATG
GATGACCTCCTGACTTCCTACATTAGCCAGATGCTCACAGCCATGAGCAAACAGCGGGGCTCCAGGAGCG
GCAAGTGAACAGTCACGGGGAGGTGCTGGTTCCATGCCTGCTCTCGAGGCAGCAGTGGGTTCAGGCCCAT
CAGCTACCCCTGCAGCTGGGGAAGACTTATGCCATCCCGGCAGCGAGGCTGGGCTGGCCAGCCACCACTG
ACTATACCAACTGGGCCTCTGATGTTCTTCCAGTGAGGCATCTCTCTGGGATGCAGAACTTCCCTCCATC
CACCCCTCTGGCACCTGGGTTGGTCTAATCCTAGTTTGCTGTGGCCTTCCCGGTTGTGAGAGCCTGTGAT
CCTTAGATGTGTCTCCTGTTTCAGACCAGCCCCACCATGCAACTTCCTTTGACTTTCTGTGTACCACTGG
GATAGAGGAATCAAGAGGACAATCTAGCTCTCCATACTTTGAACAACCAAATGTGCATTGAATACTCTGA
AACCGAAGGGACTGGATCTGCAGGTGGGATGAGGGAGACAGACCACTTTTCTATATTGCAGTGTGAATGC
TGGGCCCCTGCTCAAGTCTACCCTGATCACCTCAGGGCATAAAGCATGTTTCATTCTCTGGCC

The expression plasmid pCMV-Sport6-Myo7a (a kind gift from Manuel Taft, Institute for Biophysical Chemistry, Hannover Medical School, Hannover, Germany) served as the template for the PCR reaction. The myosin-VIIA sequence was cloned into a state-of-the-art 3^{rd} generation, self-inactivating (SIN) LV vector harboring an internal spleen focus forming virus (SFFV) promoter by exchanging the transgene for myosin-VIIA using the SgrAI (for myosin-VIIA)/AgeI (in the vector backbone) restriction sites, which create compatible ends, at the 5′ end and the Sall restriction site at the 3′ end. As sequence verification by Sanger sequencing identified point mutations and a small deletion as compared to the reference sequence, this part of the MYO7A coding sequence was exchanged for the correct sequence ordered as a gene synthesis product (Twist Biosciences, South San Francisco, CA, USA) and inserted through restriction enzyme based cloning. A reporter cassette consisting of a dTomato transgene behind an internal ribosomal entry site (IRES) and flanked by a SalI restriction site at either end was inserted behind the myosin-VIIA transgene using the SalI restriction site, generating the final construct pRRL.PPT.SF.MYO7A.i2.dTomato.pre (LV-MY07A). A control vector only expressing the dTomato reporter driven by an SFFV promoter was generated by inserting the dTomato sequence flanked by Agel and Sall into the vector backbone using the unique Agel and Sall restriction sites, generating pRRL.PPT.SF.dTomato.pre (LV-ctrl). For in-vivo application, vector counterparts harboring a short version of the hybrid cytomegalovirus (CMV) enhancer fused to the chicken beta-actin promoter (CAGs) to drive expression of the transgene cassette were cloned by exchanging the SFFV promoter for CAGs using NheI and SgrAI (LV-MYO7A) or XhoI and AgeI (LV-ctrl) restriction sites.

### Cell Culture

Human embryonic kidney HEK293T cells and human fibrosarcoma HT1080 cells were cultured at 37 °C and 5% CO₂ in Dulbecco's Modified Eagle Medium (DMEM; Gibco®, Thermo Fisher Scientific, Langenselbold, Germany) supplemented with 10% heat-inactivated fetal bovine serum (h.i. FBS), 1 mM sodium pyruvate, and 100 U/mL penicillin and 100 μg/mL streptomycin (all PAN Biotech, Aidenbach, Germany). Murine House Ear Institute-Organ of Corti 1 (HEI-OC1) cells were kindly provided by Dr. Federico Kalinec (UCLA Head and Neck Surgery, Los Angeles, CA, USA) and cultured under permissive conditions at 33 °C and 10% CO₂ in DMEM supplemented with 10% h.i. FBS, 1 mM sodium pyruvate, and 100 U/mL penicillin (Sigma-Aldrich Biochemie GmbH, Hamburg, Germany). All cell lines were passaged every 2-3 days using trypsin (PAN Biotech)-assisted detachment.

### Production of Viral Vector Particles

Viral vector particles were produced as described previously [37]. In brief, HEK293T cells were seeded at 5×10⁶ per 10 cm dish the day prior to transfection. Calcium phosphate precipitation was performed combining 5 μg of the transfer vector plasmid, 6 μg of an expression plasmid for Rev, 12 μg of a lentiviral gag/pol expression plasmid and 1.5 μg of a plasmid encoding for VSV-G. Transient transfection of the HEK293T cells with the DNA mixture was then performed in the presence of 15 mM HEPES (PAN Biotech) and 25 μM chloroquine (Sigma-Aldrich). The medium was exchanged to standard culture medium additionally supplemented with 15 mM HEPES at 6-12 h post-transfection. Supernatants containing viral vector particles were collected at 36 h and 48 h post-transfection, filtered through 0.22 μm pore size filters and concentrated 100-fold by ultracentrifugation for 2 h at 25000 rpm (rotor SW32Ti; Beckman Coulter GmbH, Krefeld, Germany) and 4 °C. The viral vector preparations were resuspended in PBS with 15 mM HEPES and stored at −80 °C until further usage.

### Transduction and Titer Determination

Transduction was performed in the respective culture medium assisted by 4 μg/mL protamine sulfate (Sigma-Aldrich). For determination of viral vector titers, HT1080 cells were seeded at 7×10⁴ cells per well in a 12-well format. On the following day, the medium was exchanged to contain protamine sulfate, viral supernatants were added at different volumes and spin inoculated for 1 h at 863 xg and 32-37 °C. Three wells were harvested for counting to determine the cell number at the time point of transduction. Cells were passaged 2-3 times until analysis by flow cytometry. For this, the cells were harvested using trypsin, pelleted by centrifugation for 5 min at 400 xg and resuspended in FACS buffer consisting of 2% FBS and 2 mM EDTA (Ambion®, Thermo Fisher Scientific) in PBS. Samples were processed on a CytoFLEX S flow cytometer (Beckman Coulter, Krefeld, Germany) and analyzed using FlowJo software (Tree Star, Inc., Ashland, OR, USA). Titers were calculated based on the volume of viral vector supernatant applied, the cell number at transduction, and the percentage of cells expressing the vector-encoded dTomato reporter protein at flow cytometry analysis. Titers are expressed as transducing units per milliliter (TU/mL).

### Transduction of HEI-OC1 Cells

HEI-OC1 cells were seeded at 3×10⁴ per well of a 24-well plate on the day prior to transduction. Three wells were harvested for counting to determine the cell number at the time point of transduction, and the volume of viral vector supernatant was calculated based on the vector's titer to apply defined multiplicities of infection (MOI), i.e. a defined particle number per seeded cell. The transduction procedure followed the same protocol as described under titration. The percentage of cells expressing the vector-encoded dTomato reporter protein was assessed by flow cytometry as described under titration.

### Intracellular Staining for Flow Cytometry

Cells were harvested using trypsin-assisted detachment and pelletized by centrifugation for 5 min at 400 xg. The pellets were resuspended in 500 μL Fixation Buffer (Cat #420801, BioLegend, San Diego, CA, USA) and cells incubated for 20 min at room temperature. Samples were pelletized again and washed with 1 mL FACS buffer, followed by three cycles of resuspension in 1x Intracellular Staining Perm Wash Buffer (Cat #421002, BioLegend) and centrifugation for 5 min at 400 xg. Incubation with the primary antibody polyclonal rabbit-anti-myosin-VIIA (Catalog #25-6790, Proteus BioSciences Inc., Ramona, CA, USA) was performed at 1:300 dilution in 1x Intracellular Staining Perm Wash Buffer for 20 min at room temperature, followed by two washes with 1x Intracellular Staining Perm Wash Buffer. Incubation with the secondary antibody Alexa Fluor® 488 AffiniPure Donkey Anti-Rabbit IgG (H+L) (Catalog #711-545-152, Jackson ImmunoResearch Europe Ltd, Ely, UK) was performed at 1:800 dilution in 1x Intracellular Staining Perm Wash Buffer for 20 min at room temperature in the dark. After two washes with 1x Intracellular Staining Perm Wash Buffer, cell pellets were resuspended in FACS buffer, processed on a CytoFLEX S flow cytometer and analyzed using CytExpert software.

### Immunofluorescence of Cultured Cells

To investigate myosin-VIIA expression in cultured cells, vector-transduced or non-transduced HEI-OC1 cells were seeded onto 24-well plastic plates in standard culture medium 1-2 days prior to analysis. At confluence above 50%, the cells were rinsed in phosphate-buffered saline (PBS, PAN Biotech) followed by fixation for 15 min in 4% paraformaldehyde (Electron Microscopy Sciences, Science Services GmbH, Munich, Germany). Cells were permeabilized for 10 min in 0.2% Triton-X-100 (Sigma-Aldrich) in PBS(=PBT) and then incubated for 30 min in 5% FBS in PBS. Incubation with the primary antibody polyclonal rabbit-anti-myosin-VIIA (Catalog #25-6790, Proteus BioSciences Inc.) was performed overnight at 4 °C and 1:300 dilution in reaction buffer (RB) containing 0.5% bovine serum albumin (PAN Biotech) in 0.1% Triton-X-100 in PBS. The following day, the cells were rinsed once in washing buffer (WB, 0.1% Triton X-100 in PBS), followed by two further washes of 10 min each in WB. Incubation with the secondary antibody Alexa Fluor® 488 AffiniPure Donkey Anti-Rabbit IgG (H+L) (Catalog #711-545-152, Jackson ImmunoResearch Europe Ltd) was performed at 1:800 dilution in RB for 1 h at room temperature in the dark. After one short and one 10 min wash in WB, the cells were kept in PBS until analysis using an Axio Observer microscope (Carl Zeiss A G, Oberkochen, Germany).

### Animals/Ethics

C57BL/6 mice and Shaker-1 Sh1/LeJ mice were purchased from Jackson Laboratory (Bar Harbor, ME, USA) and maintained in a breeding colony. All animal care and procedures were approved by the Institutional Animal Care and Use Committee University of Kansas University Medical Center. Studies on effects of LV-MYO7A in normal hearing mice were carried out in C57BL/6 mice. Evaluation of MYO7A gene therapy was tested in Shaker-1 mice aged P4-P270. Outcomes measures included hearing testing using auditory evoked brain stem responses (ABR), balance testing using rotarod and actimeter testing, and histology/immunofluorescence staining.

### Genotyping

Genomic DNA was extracted from mouse pinna by incubating ear punches with 40 μL tissue preparation solution (Sigma, Cat No. T3073) and 40 μL extraction solution (Sigma, E7526) at 55 °C for 30 minutes. This was followed by incubating the mixture at 90 °C and for 5 minutes then adding 10 μL neutralization solution (Sigma, N3910). Genotype was then determined by PCR assay adapted from Self et al.¹². The primers CATGTCCAAGGTCCTCTTCC (SEQ ID NO: 16) forward and CCTAGAATCAGTGCAGAGCA (SEQ ID NO: 17) reverse were designed to amplify a DNA fragment across the mutation, annealing at 58 °C with Dream Taq DNA polymerase (Thermo Fisher Sci., EP0702), dNTP mix (Thermo-Sci, FERR0192), followed by an MspI digest to give a 328 bp product in homozygous mutants, 189 bp and 139 bp products in wild type, and 328 bp, 189 bp and 139 bp products in heterozygous littermates. Homozygous mutant mice are designated as Shaker-1^{mut} (−/−) and heterozygous mutant mice as Shaker-1^{het} (+/−) mice to clarify which allele group is being evaluated.

### Delivery of Vector to the Posterior Semicircular Canal Through Canalostomy

Canalostomy was performed as previously established in Schlecker, C. et al., (2011). Gene Ther 18, 884-890. Adult C57BL/6 mice aged 1 month (average weight: 11 g) or Shaker-1+/+, +/− or −/− mice aged 16 days were anesthetized with an intraperitoneal (IP) injection of a mixture of ketamine (150 mg/kg), xylocaine (6 mg/kg) and acepromazine (2 mg/kg) in sodium chloride 0.9%. A dorsal postauricular incision was made, and the posterior semicircular canal exposed. Using a microdrill, a canalostomy was created, exposing the perilymphatic space. Subsequently, 1 μL of vector was injected using a Hamilton microsyringe with 0.1 μL graduations and a 36 gauge needle. The canalostomy was sealed with bone wax, and the animals were allowed to recover. A single batch of LV-MYO7A vector preparation with a titer of 7.78×10⁶ TU/mL was used for all in-vivo experiments.

### Anesthesia and Posterior Canal Delivery in P4 Shaker-1 Mice

P4 Shaker-1 mice were anesthetized with cold as described in Isrig et al.⁴¹. The posterior semicircular canal was exposed in the postauricular space and a sharp pick was used to open the canal. Vector particles at a dose of 1 μL (7.78×10⁶ TU/mL) were injected using a microsyringe as described above. Genotyping was carried out at P21 as described.

### Histology and Immunohistochemistry

Mice were anesthetized with intraperitoneal applications of phenobarbital (585 mg/kg) and phenytoin sodium (75 mg/kg) (Beuthanasia®-D Special, Schering-Plough Animal Health Corp., Union, NJ, Canada) and sacrificed via intracardiac perfusion with 4% paraformaldehyde in phosphate buffered saline (PBS). The temporal bones were removed and trimmed. The stapes was removed and the round window was opened with a needle. The temporal bones were postfixed overnight in 4% paraformaldehyde in PBS at 4 °C. After rinsing in PBS three times for 30 min, the temporal bones were decalcified in 10% EDTA (ethylene diamine tetraacetic acid) for 48 h. For histology, the temporal bones were rinsed in PBS and embedded in paraffin. Seven μm sections were cut in parallel to the modiolus, mounted on Fisherbrand® Superfrost®/Plus Microscope Slides (Fisher Scientific, Pittsburgh, PA, U.S.A.) and dried overnight. Samples were deparaffinized and rehydrated in PBS two times for 5 min, then three times in 0.2% Triton X-100 in PBS for 5 min and finally in blocking solution 0.2% Triton X-100 in PBS with 10% fetal bovine serum for 30 min at room temperature. After blocking, specimens were treated with anti-annexin V rabbit polyclonal antibody (Abcam cat ab 182646) diluted 1:100 in blocking solution. The tissue was incubated for 48 h at 4 °C in a humid chamber. After three rinses in 0.2% Triton X-100 in PBS, immunofluorescent detection was carried out with anti-rabbit IgG (1:50; Alexa Fluor 488 nm; Invitrogen®Inc.). The secondary antibody was incubated for 6 h at room temperature in a humid chamber. The slides were rinsed in 0.2% Triton X-100 in PBS three times for 5 min and finally coverslipped with ProLong® Gold antifade reagent (Invitrogen™ Molecular Probes, Eugene, OR, U.S.A.). For whole mount analysis, cochleae were harvested at one week post-vector-injection and decalcified as described above. Clearing and whole mount imaging of the dTomato signal was then carried out as described in Risoud et al.⁴².

For organ of Corti preparation, the decalcified otic capsule was carefully removed and the organ of Corti dissected away from the modiolus. For utricle preparation, the decalcified bone between the oval and round windows was removed. The macular organs were identified and the utricle removed. Residual otolith debris was brushed off the neuroepithelium. The tissue was then incubated in PBS+0.1% Triton X-100 for 10 minutes followed by a 10 min incubation in phalloidin-FITC (Abcam ab235137) diluted 1:80 in PBS+0.1% triton x-100 at room temperature. The tissue was then washed 3 times for 10 min in PBS and mounted in Vectashield Plus mounting medium (Vector Labs, Newark C A) between two coverslips. Quantification of vestibular hair cells was carried out by placing a counting grid over 5 areas of each utricle and counting total stereocilia-bearing hair cells and total stereocilia-bearing cells that expressed dTomato.

Confocal imaging was carried out on a Nikon TI2-E inverted microscope with perfect focus attached to a Yokagawa CSU-W1 spinning disk confocal system with SoRa super-resolution or a Nikon Eclipse TiE inverted motorized microscope with perfect focus attached to an AIR-SHR confocal system. Images were analyzed using Nikon Elements software and scale bar determination for individual images was completed in Adobe Photoshop 21.0.1 based on exemplary scale bar measurements in the source data.

### Evaluation of Cochlear Hair Cell Survival

Midmodiolar sections were evaluated for the presence of auditory sensory hair cells within the apical and basal turns of the cochlea. Within each chamber, the presence or absence of an inner hair cell and three outer hair cells were noted. Positive hair cell counts were made if the inner and outer hair cells were appropriately located within the organ of Corti and possessed normal nuclear staining. The total number of inner and outer hair cells was determined for all treatment groups (N=5 mice per group) across all sections (N=5 sections per mouse); the percentage of hair cells present was then calculated for the entire group (number of hair cells found divided by the expected number x 100). This method is not intended to determine the absolute number of sensory hair cells but rather a qualitative evaluation of sensory hair cells throughout multiple regions of the cochlea.

### ABR Measurement

ABR thresholds were recorded using the Intelligent Hearing Systems Smart EP program (IHS, Miami, FL, U.S.A.). Animals were anesthetized as described above and kept warm on a heating pad (37 °C). Needle electrodes were placed on the vertex (+), behind the left ear (−) and behind the opposite ear (ground). Tone bursts were presented at 4, 8, 16 and 32 kHz, with duration of 500 μs using a high frequency transducer. Recording was carried out using a total gain equal to 100K and using 100 Hz and 15 kHz settings for the high and low-pass filters. A minimum of 128 sweeps was presented at 90 dB SPL. The SPL was decreased in 10 dB steps. Near the threshold level, 5 dB SPL steps using up to 1024 presentations were carried out at each frequency. Threshold was defined as the SPL at which at least one of the waves could be identified in two or more repetitions of the recording. The preoperative threshold was measured in P16 animals prior to the first operation and the final postoperative threshold was measured before sacrificing the animals. P4 treated animals did not receive pretreatment hearing screens. We tested mice prior to vector delivery and at P30 or P180.

### DPOAE Measurement

To evaluate the functional damage on the OHC, distortion product otoacoustic emissions (DPOAE) were recorded on both sides using the IHS Program described above. The distortion products were measured for pure tones from 2 kHz to 32 kHz using the IHS high frequency transducer. The Etymotic 10B+ Probe was inserted to the external ear canal. L1 Level was set to 65 dB, L2 Level was set to 55 dB. Frequencies were acquired with an F2-F1 ratio of 1.22 using 16 sweeps. Nine stimulus levels ranging from 65 dB SPL to 31 dB SPL were used in 5 dB steps.

### Rotarod

To evaluate balance function mice were tested on a Rotarod treadmill (ENV-575M, Med Associated Inc., Georgia, USA). The rod started at an initial velocity of 4 rpm and accelerated to 40 rpm at 100 sec. The time from the start of acceleration, until the mouse fell completely off the rod, was recorded. The test was stopped after a maximum of 200 sec. All mice were trained on the rod for two days prior to the baseline recording.

### Force-Plate Actimetry

BASi Force-Plate Actimeter (FPA) (Bioanalytical Systems, Inc., Mount Vernon, IN) was used to test locomotor activity and exploratory behaviors of the mice. Mice were placed into the testing arena and allowed to move freely for 10 minutes (60 frames) and total distance traveled, area traveled, left and right turns, and total degrees turned were recorded and analyzed by FPA Analysis.

### Statistics

P-values of <0.05 were considered significant. Statistical analysis was performed with Prism v9.0 using ANOVA for repeated measures, if not stated otherwise in the figure legends. P-values of <0.05 were considered significant. **** p≤0.001; *** p≤0.005; ** p≤0.01; * p≤0.05.

### Example 2: Lentiviral Vectors are Able to Package, Transfer and Express Native Myosin VIIA (LV-MYO7A)

To establish a gene therapeutic option for USH1B, a state-of-the art and high-capacity 3^{rd} generation lentiviral vector (LV) was equipped with the native 6645 bp sequence of the human myosin VIIA cDNA (MY07A) (SEQ ID NO: 1) canonical isoform. The vector harbored a self-inactivating (SIN) architecture devoid of the enhancer and promoter elements naturally present in the U3 region of the the long-terminal repeats (LTRs). This design confers an improved safety profile by reducing the risk of insertional mutagenesis, and allows the usage of an internal promoter of choice to drive transgene expression.

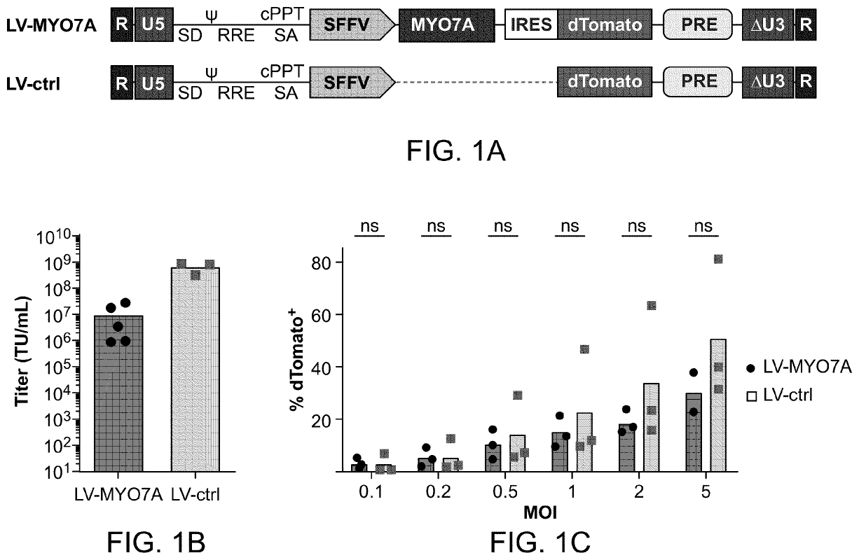

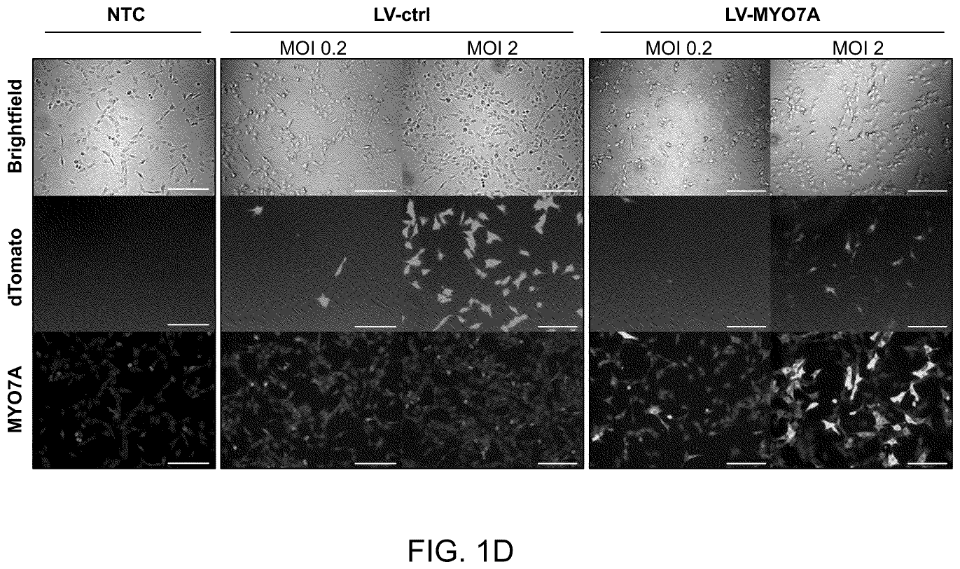

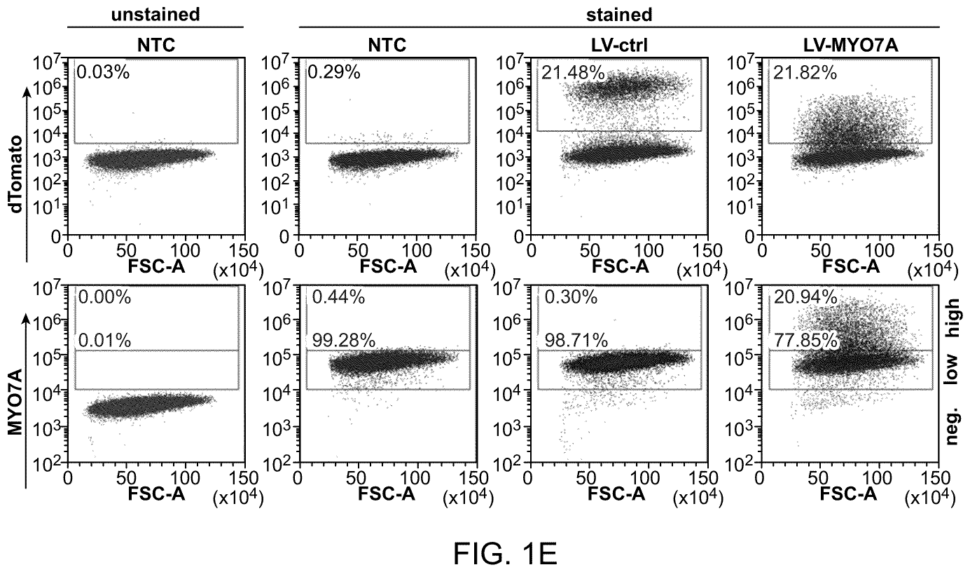

Here, an internal spleen focus forming virus (SFFV) promoter as a ubiquitously active and reportedly strong promoter was chosen to mediate high-level and sustained expression of the transgene cassette. To facilitate titration of viral vector particle preparations and identification of successfully transduced cells upon in-vitro and in-vivo administration, the MYO7A cDNA was linked to a dTomato reporter gene via an internal ribosomal entry site (IRES) to create the lentiviral vector LV-MYO7A (FIG. 1A). A counterpart expressing dTomato only served as a reference and control (LV-ctrl).

Transient production using a split-packaging system successfully generated lentiviral particles despite the challenging size of the MYO7A cDNA (FIG. 1B). Although titers were 1-2 log lower for LV-MYO7A as compared to LV-ctrl (which an average of 10⁷ TU/mL) they were in a range that is sufficient for in-vitro and in-vivo application.

To evaluate vector functionality and the capacity to transduce inner ear cells, LV-MYO7A was tested for its in-vitro performance using the established hair-cell-like cell line HEI-OC1 (27). Upon transduction at different multiplicity of infection (MOI), i.e. applying defined numbers of viral vector particles per seeded cell, no significant difference in the percentage of successfully transduced, dTomato-positive cells was observed by flow cytometry analysis between LV-MYO7A and LV-ctrl across all MOIs tested (FIG. 1C). This confirmed that the transduction efficiency of the lentiviral vector encoding the large MYO7A cDNA was comparable to smaller vectors.

Staining using an anti-myosin-VIIA antibody followed by immunofluorescence microscopy or flow cytometry revealed low-level endogenous MYO7A expression in the non-transduced HEI-OC1 cells and no signal for dTomato (FIGS. 1D-1E). The shift of the cell populations in the Myo7a channel of flow cytometry plots seen upon antibody addition could not be confirmed as true Myo7a protein expression, but rather represented a staining artifact. In cells transduced with LV-ctrl, MYO7A signal was unaltered as compared to the non-transduced cells, but a high signal for dTomato was present.

In contrast, in HEI-OC1 cells transduced with LV-MYO7A, a clear MYO7A-positive cell population was detected. Furthermore, the same cells showing a high MYO7A signal were also positive for dTomato, indicating successful co-expression of MYO7A and the dTomato reporter from the vector. Of note, dTomato expression levels from LV-ctrl were higher than from LV-MYO7A. This is in line with reports on reduced expression of the second gene in bicistronic vectors that use IRES elements (28), as it is the case for LV-MYO7A but not LV-ctrl, and thus does not indicate any disadvantage or inferior performance of LV-MYO7A. Altogether, despite the challenging size of the MYO7A transgene, fully functional LV vector particles could be produced that successfully transferred MYO7A and expressed the protein in inner-ear-derived, hair-cell-like HEI-OC1 cells.

### Example 3: LV-MYO7A Efficiently Transduces the In-Vivo Cochlea and does not Disturb Hearing Function in Normal-Hearing Mice

Before assessment of the therapeutic effects of LV-MYO7A in the Shaker-1 model, the potential of the employed 3^{rd} generation LV vector platform to transduce the inner ear was investigated as, until now, LV vector application to the inner ear has generally been ineffective and limited to few cell populations, which importantly did not include hair cells.¹⁹⁻²¹ For all in-vivo applications, LV-ctrl and LV-MYO7A were equipped with the short version (CAGs) of the CAG promoter (a hybrid of the cytomegalovirus enhancer fused to the chicken beta-actin promoter), which is less prone to silencing than the SFFV promoter. To determine transduction of the mouse inner ear, 1 μL of VSV-G pseudotyped LV particles was injected into 1-month-old wild-type, normal-hearing C57BL/6 mice through canalostomy via the posterior semicircular canal (PSCC).

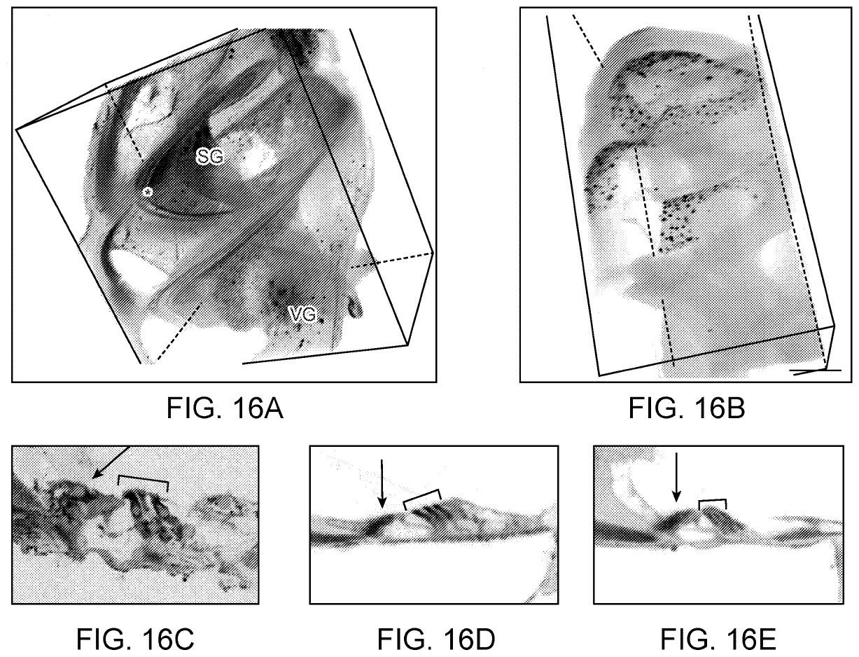

3D reconstruction of the dTomato reporter signal in whole cleared cochleae demonstrated efficient transduction of multiple cell types, including hair cells and spiral ganglion cells, throughout all cochlear turns, as well as transduction of the vestibular ganglion (FIG. 11A and FIGS. 16A-16C). Cochlear cross sections confirmed dTomato expression in the spiral ganglion as well as in inner and outer hair cells in multiple turns of the cochlea upon application of the small LV-ctrl vector (FIG. 11B and FIGS. 16D-16E). This confirmed that the employed vector platform can efficiently transduce the USH1B-relevant target cells, i.e. inner and outer hair cells, in the cochlea. Importantly, injection of 1 μL of LV-MYO7A, despite the challenging vector size and the associated lower titer (7.78×10⁶ TU/mL), also achieved dTomato expression in both inner and outer hair cells as well as spiral ganglion cells in multiple cochlear turns (FIG. 11C and FIGS. 17A-17D). Whole mount organ of Corti preparation furthermore demonstrated a high efficiency of hair cell transduction using LV-MYO7A, with up to 100% of the inner and outer hair cells covered by the whole mount showing dTomato signal (FIG. 11D and FIGS. 18A-18P). Notably, the structure of the tissue in the cochlea was preserved upon LV vector administration, without any signs of degeneration (FIGS. 11A-11D and FIGS. 16-18).

To determine if any potential negative functional effects resulted from the canalostomy approach used for in-vivo vector administration and/or from ectopic MYO7A overexpression, hearing function was assessed in the injected C57BL/6 mice at one week post-administration of LV-MYO7A. ABR recordings revealed no difference in the hearing thresholds of the same C57BL/6 mice pre- and post-treatment with LV-MY07A (FIG. 11E). Altogether, the dTomato signal indicated activity of the CAGs promoter and expression of the transgene cassette from LV-MYO7A in the cochlea. The characteristics of successful packaging of LV-MYO7A and the high efficiency of LV transduction of cochlear hair cells in the absence of adverse effects indicate LV-MYO7A to be a suitable candidate for in-vivo gene therapy of USH1B.

### Example 4: Vestibular Hair Cells are a Target for LV-MYO7A Gene Therapy

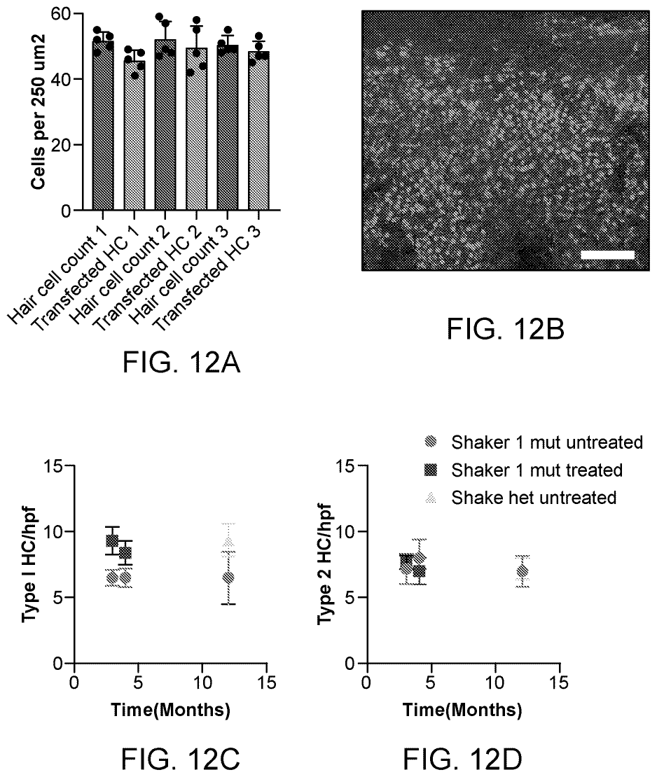

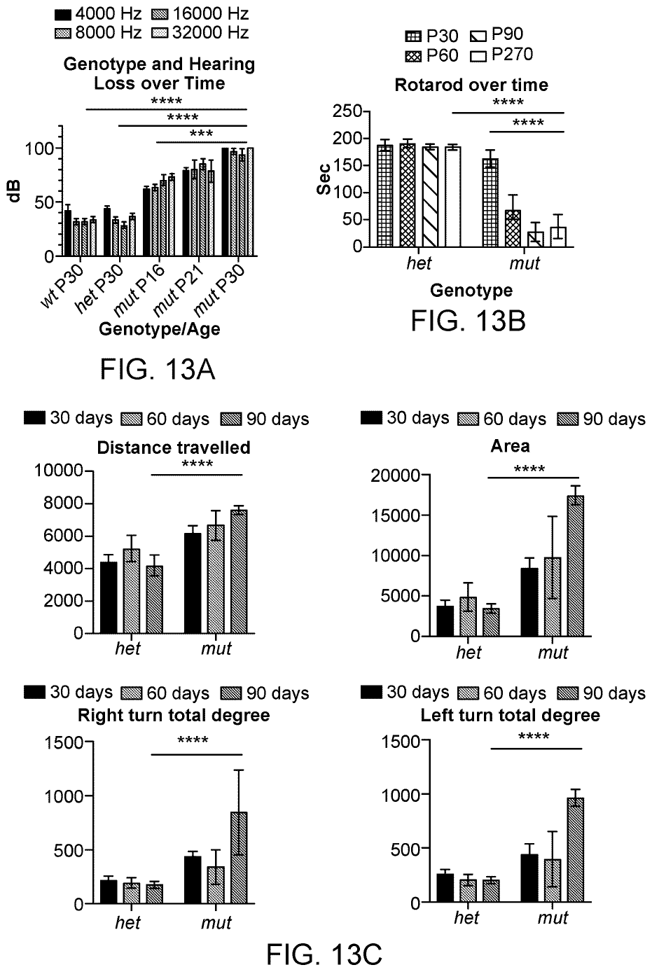

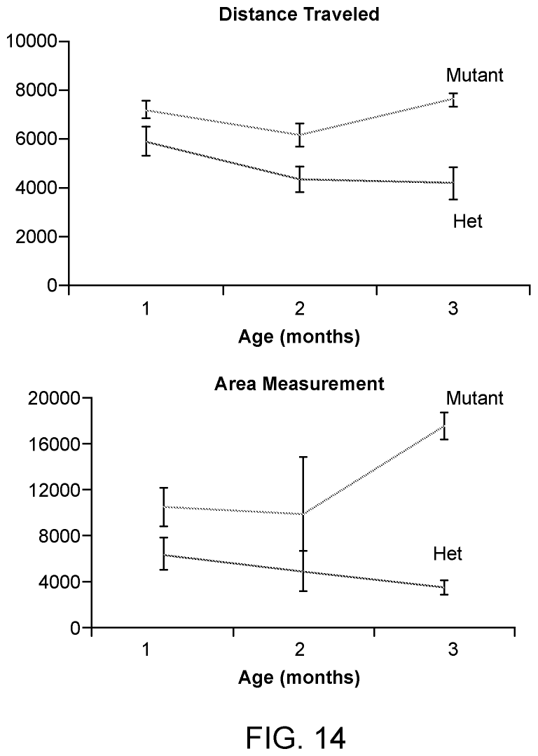

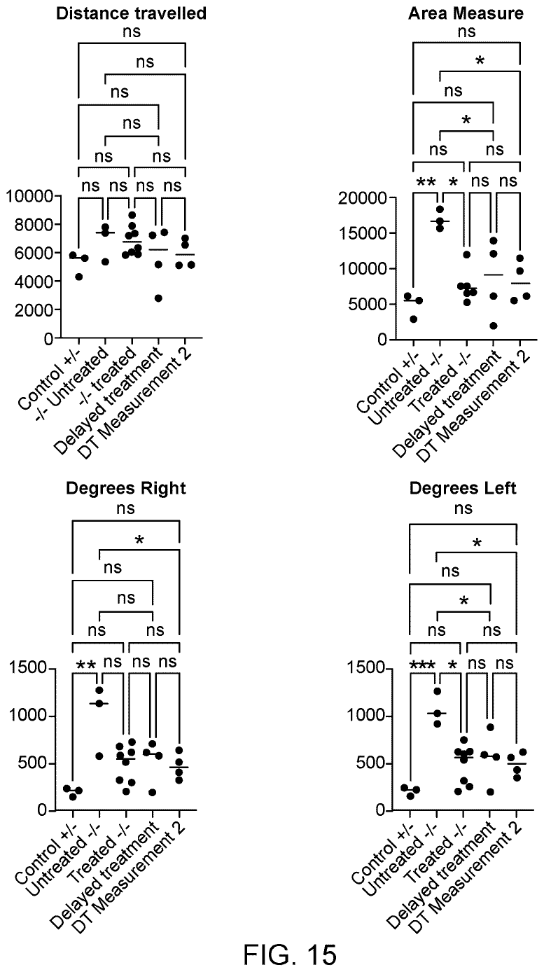

Besides hearing loss, inner ear-related deficits in USH1B patients and homozygous Shaker-1 mice include severe imbalance. Therefore, the employed 3^{rd} generation LV vector platform was also characterized for its potential to transduce the vestibular organ of wild-type C57BL/6 mice. Injection of 1 μL of LV-MYO7A into 1-month-old mice resulted in successful transduction of the vestibular neuroepithelium, incl, the vestibular ganglion (FIG. 11A and FIGS. 16A-16B), the utricle, and the saccule (FIGS. 12A-12B), as indicated by dTomato expression. Quantification of dTomato-positive cells in the utricle revealed a high transduction efficiency ranging from 89% to 95% among the hair cells, which were identified through phalloidin-assisted labeling (FIG. 12C). As before for the cochlea, the structure of the vestibular tissue was normal and without any signs of damage or degeneration (FIG. 12A).

Any potential negative effects of the canalostomy and/or of MY07A overexpression in the vestibular organ were investigated through functional testing of the balance behavior by rotarod analysis. Rotarod times recorded for the mice did not change due to LV-MYO7A treatment, with similar balance function of the mice pre- and post-LV-MYO7A-treatment (FIG. 12D).

In the Shaker-1 mouse model, injection of 1 μL of LV-MYO7A at P16 also did not cause any degeneration of vestibular hair cells, with similar or even higher type-I and type-II hair cell counts in the treated versus non-treated mice as determined at 3 and 4 months of age (FIG. 12E). Importantly, the numbers of vestibular hair cells in untreated homozygous Shaker-1^{mut} animals as counted in serially sectioned utricles at 3, 4 and 12 months of age remained stable, and type-II hair cell numbers were not different from the utricles of heterozygous Shaker-1^{het} littermates. This implicates the presence of a cellular target for LV-MY07A gene therapy even at time points far beyond the onset of functional impairment, and together with efficient LV vector transduction indicates vestibular hair cells as a promising target for LV-MYO7A gene therapy.

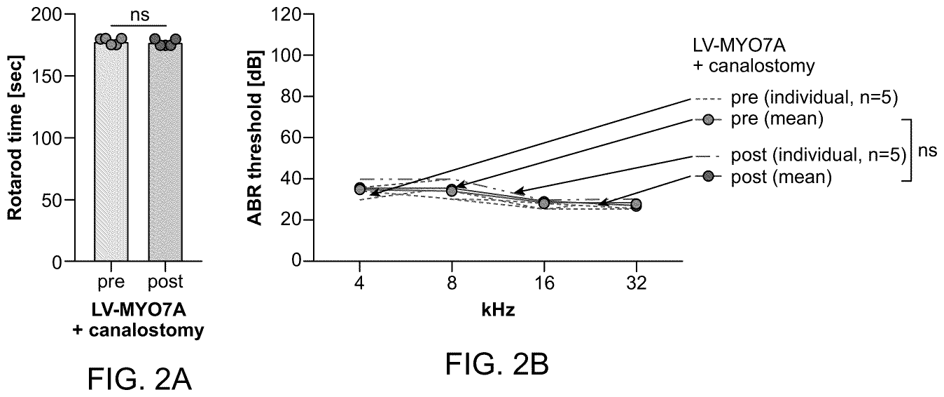

Vestibular function was assessed by rotarod analysis, which determines the time that the mice remain balanced on a rotating rod without falling off, with a maximum test duration of 200 seconds. Rotarod times of the mice did not change through LV-MYO7A treatment, with similar balance function of the mice pre- and post-treatment (FIG. 2A). Hearing was determined through auditory brainstem recording (ABR) in response to 4—, 8-, 16- and 32-kHz tone bursts, which revealed no difference post-treatment with LV-MYO7A as compared to the hearing performance of the same mice pre-treatment (FIG. 2B). Thus, LV-MYO7A application did not result in any adverse effect on balance or hearing function. The characteristics of successful packaging and efficient in-vivo delivery of MYO7A in the absence of adverse effects indicate LV-MYO7A to be a suitable candidate for in-vivo gene therapy of USH1B.

Vestibular function was assessed by rotarod analysis, which determines the time that the mice remain balanced on a rotating rod without falling off, with a maximum test duration of 200 seconds. Rotarod times of the mice did not change through LV-MYO7A treatment, with similar balance function of the mice pre- and post-treatment (FIG. 2A). Hearing was determined through auditory brainstem recording (ABR) in response to 4-, 8-, 16- and 32-kHz tone bursts, which revealed no difference post-treatment with LV-MY07A as compared to the hearing performance of the same mice pre-treatment (FIG. 2B). Thus, LV-MYO7A application did not result in any adverse effect on balance or hearing function. The characteristics of successful packaging and efficient in-vivo delivery of MYO7A in the absence of adverse effects indicate LV-MYO7A to be a suitable candidate for in-vivo gene therapy of USH1B.

### Example 5: The Shaker-1 Mouse Model of USH1B: Homozygotes Show Early-Onset Loss of Balance and Hearing Loss, while Heterozygotes Show Normal Balance but Late-Onset Hearing Loss

The functional effects of LV-MYO7A gene therapy were assessed using the established Shaker-1 mouse model for USH1B, which carries a Myo7a missense mutation resulting in an R502P amino acid substitution and a highly diminished motor activity. To define the interval given for therapeutic intervention, to be able to assess the magnitude of therapeutic effects and to discriminate between the effects possible in homozygous versus heterozygous Shaker-1 mutant mice, the model was deeply characterized with regard to different genotypes and ages. For this, Shaker-1 wild-type (Shaker-1^{WT}, +/+), heterozygous mutant (Shaker-1^{het}, +/−) and homozygous mutant (Shaker-1^{mut},−/−) mice were subjected to hearing and balance testing at P16, P21, P30, P60 and/or P90, followed by histological analysis (N=5-10 per condition). A subset of Shaker-1^{mut} animals (N=5) was followed by balance analysis for 270 days and then allowed to survive to 1 year of age, at which point they were evaluated with histology.

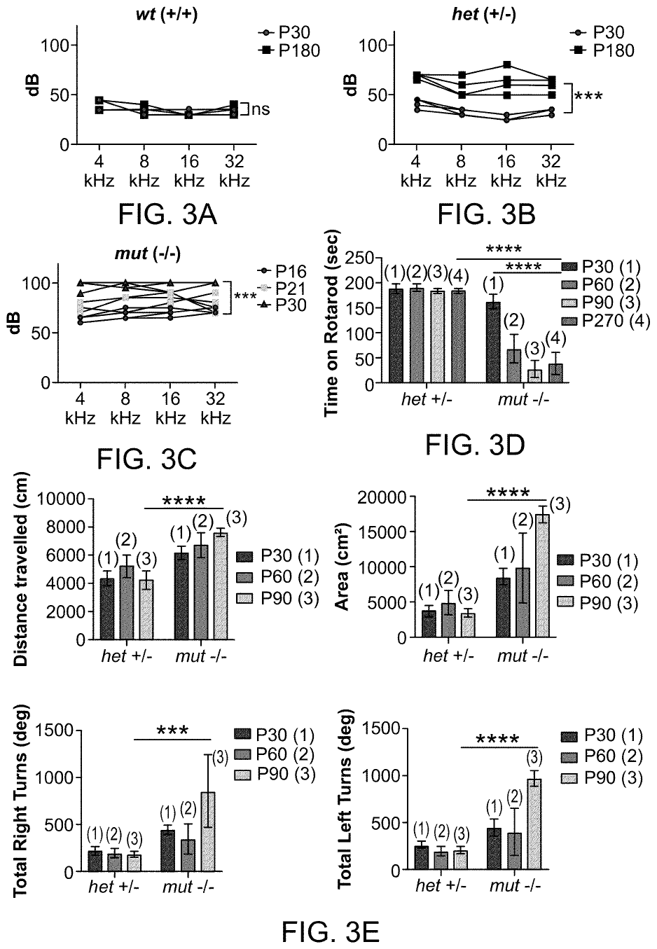

Hearing was determined through auditory brainstem recording (ABR) in response to 4—, 8—, 16-and 32-kHz tone bursts. For Shaker-1^{WT} mice, ABR measurements revealed normal thresholds of less than 45 dB across all frequencies and time points tested (FIG. 3A). Interestingly, Shaker-1^{het} mice presented with a hearing loss phenotype. At P30, the Shaker-1^{het} animals still showed normal ABR thresholds (FIG. 3B). However, hearing was observed to decline at later time points, starting between P90 and P180, resulting in hearing thresholds averaging 60-70 dB by 6 months of age. In Shaker-1^{mut} mice, in line with previous studies, hearing was already diminished but still detectable at P16, with the hearing thresholds ranging from 65 dB at the low frequencies to 75 dB at the high frequencies (FIG. 3C). Hearing loss progressed to the 80 dB range across all frequencies by P21 and declined to profound hearing loss at all frequencies by P30. Thus, homozygous Shaker-1^{mut} animals showed an early-onset hearing loss, and their Shaker-1^{het} littermates manifested with a late-onset hearing loss.

Balance function was characterized by rotarod analysis and force-plate actimetry. Rotarod analysis determines the time that the mice remain balanced on a rotating rod without falling off, with a maximum test duration of 200 seconds. Actimetry allows for tracking of the total distance traveled, the area covered during movement and the total degrees of turns as a measure of circling behavior. Shaker-1^{het} mice showed normal rotarod times throughout the three-month test period with the mice managing to stay balanced on the rod for the test duration of 200 seconds (FIG. 3D). For Shaker-1^{mut} mice, circling behavior is reported to manifest between P16 and P21. However, the mice demonstrated essentially normal time balancing on the rod at P30 (FIG. 3D). However, rotarod times then declined rapidly and progressively over the next two months, showing a significant difference to Shaker-1^{het} mice at P60 and reaching a nadir at P90. A small number of animals (n=5) were allowed to survive to nine months of age (P270) and did not show any improvement of function through repeated testing, which ruled out improvement through learning.

Force-plate actimetry tests at P30, P60 and P90 confirmed the rotarod results. Shaker-1^{het} mice never showed any changes in the total distance traveled and the area covered during movement, or any circling behavior (FIG. 3E). In contrast, Shaker-1^{mut} mice demonstrated impaired balance already at P30. In line with the rotarod results, balance function in Shaker-1^{mut} mice progressively worsened over the next 2 months in terms of the area covered and the total degrees turned, with a maximum at the final time point of analysis at P90 (FIG. 3E). Thus, profoundly impaired balance was seen in homozygous Shaker-1^{mut} animals, while heterozygous Shaker-1^{het} mice showed normal balance function.

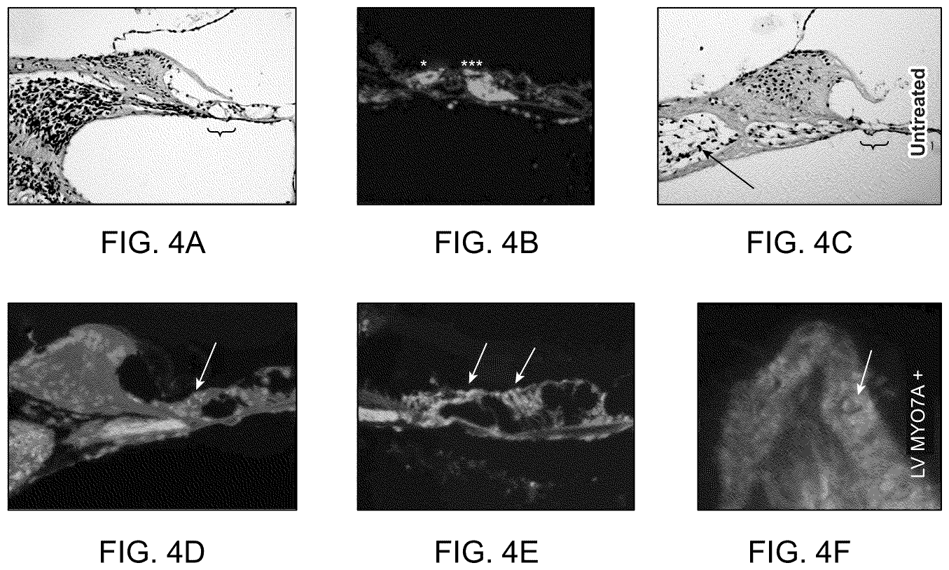

Gene therapeutic intervention requires the presence of a cellular target (i.e. the target tissue needs to be preserved at administration of the therapy), so that the time point of any potential degeneration of the cell type(s) affected by the disease defines the therapeutic window. Physiologically, MYO7A is very specifically expressed in cochlear and vestibular hair cells only, so that hair cells constitute the primary target for treatment with LV-MYO7A. Histology on cochlear sections from Shaker-1^{mut} mice demonstrated the presence of a normal-appearing organ of Corti, including the presence of hair cells, at P30 (FIG. 4A). However, at P90, both inner and outer hair cells as well as the supporting cells underneath the outer hair cells, while present, stained positive for the apoptosis marker annexin V, indicating onset of degeneration of the organ of Corti (FIG. 4B). At one year of age, Shaker-1^{mut} animals showed a complete loss of the cellular architecture of the organ of Corti, including the loss of all hair and supporting cells (FIG. 4C). Interestingly, as a secondary effect, the spiral ganglion had also degenerated at this time point. Altogether, histology confirmed the target tissue to be present after birth, making a gene therapy-based treatment concept possible up to at least P30 in the Shaker-1^{mut} mouse, when the inner ear is already physiologically nonfunctional.

### Example 6: LV-MYO7A Gene Therapy at P16 Improves Hearing and Rescues Balance Function in Homozygous Shaker-1^{mut} Mice

Having confirmed the functionality of LV-MYO7A in-vitro as well as effectiveness of the employed LV vector platform in transduction of the in-vivo inner ear, we performed an in-vivo gene therapy approach to explore the therapeutic potential of LV-MYO7A to treat the vestibulo-cochlear defects in homozygous mutant Shaker-1^{mut} mice. VSV-G pseudotyped LV-MYO7A particles were administered by injection into the posterior semicircular canal (PSCC) via canalostomy. Injections were performed at P16, i.e. after physiological hearing onset, but early enough to ensure that the targeted hair cell population was still present at intervention.

Fluorescence microscopy on cochlear sections from Shaker-1^{mut} mice at P90 showed successful transduction of inner and outer cochlear as well as vestibular hair cells with LV-MYO7A, as indicated by the signal from the vector-encoded dTomato reporter protein (FIGS. 4D-F). The dTomato signal further confirmed that the transgene cassette was still active and expressed in-vivo at 10 weeks post-vector-delivery.

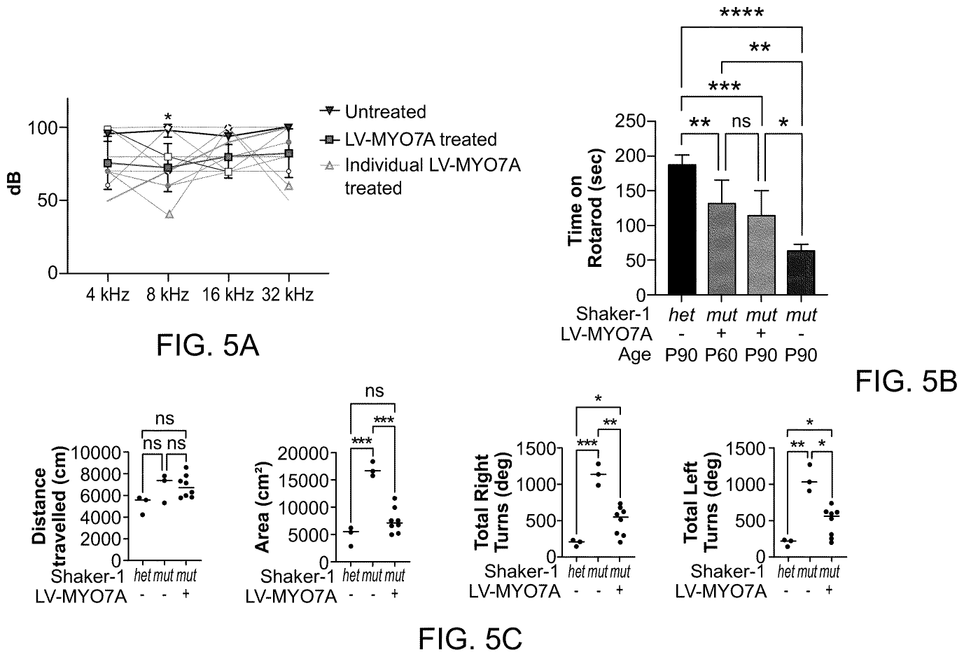

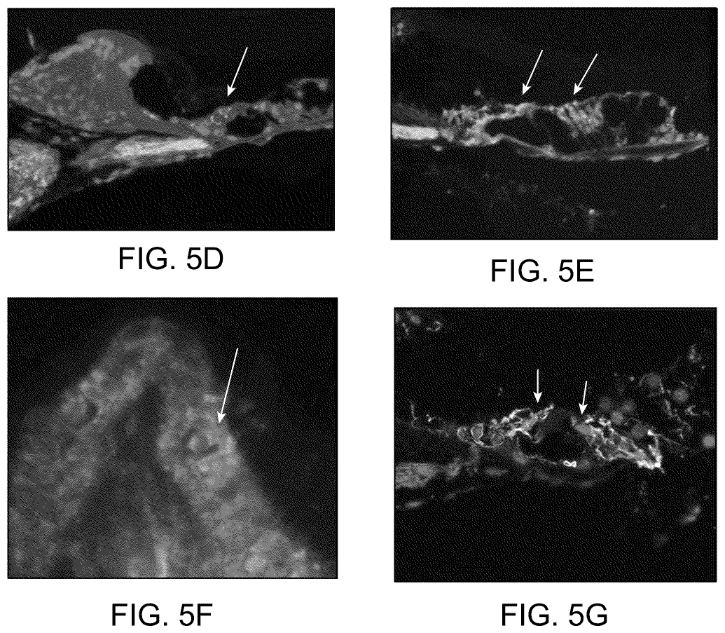

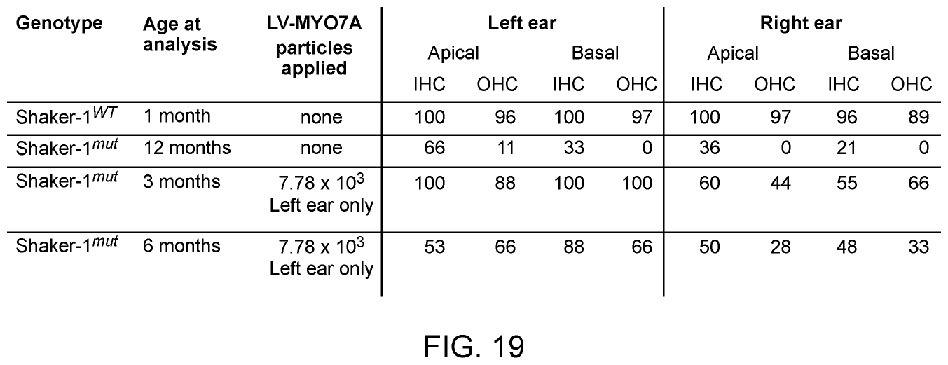

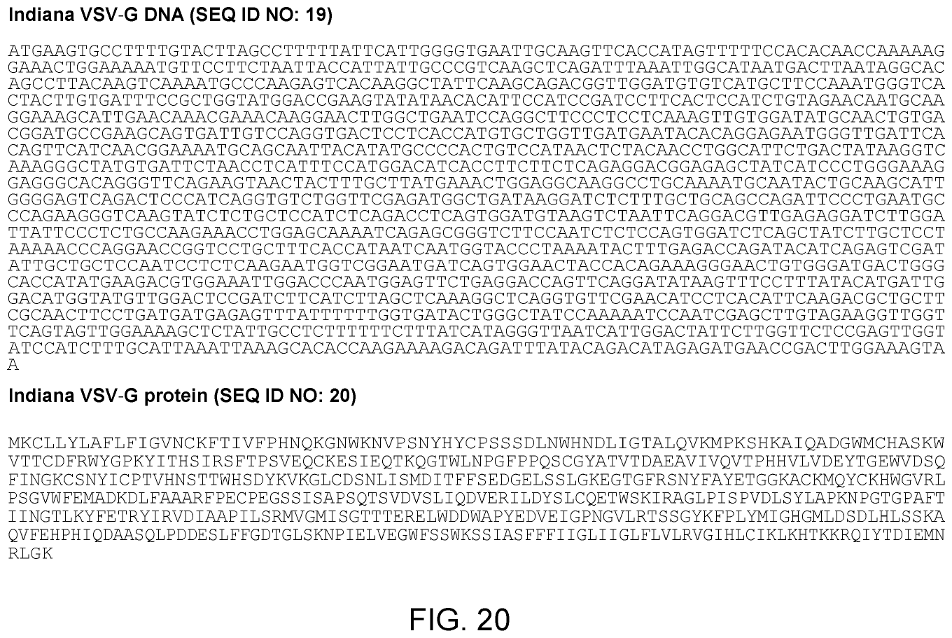

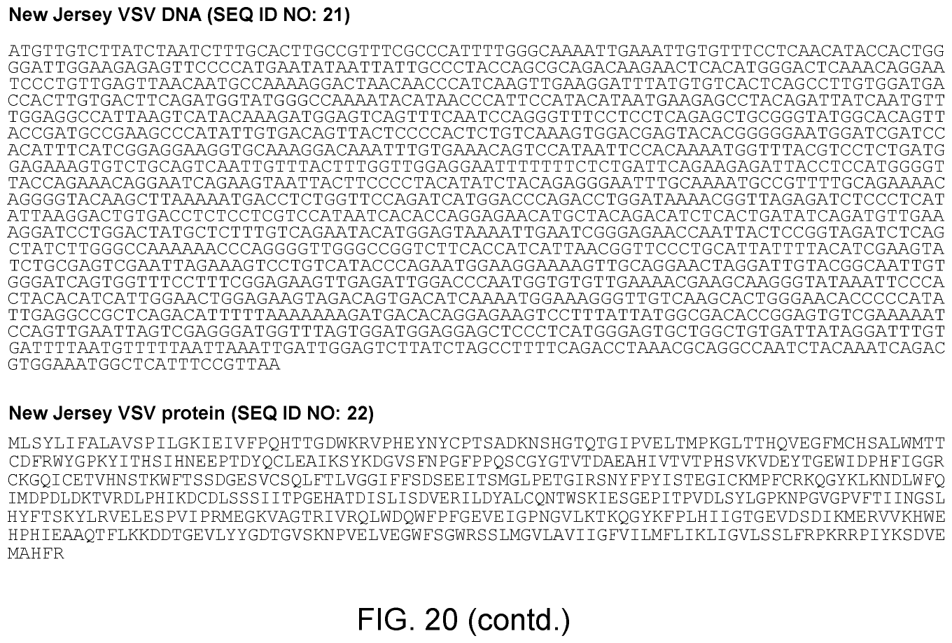

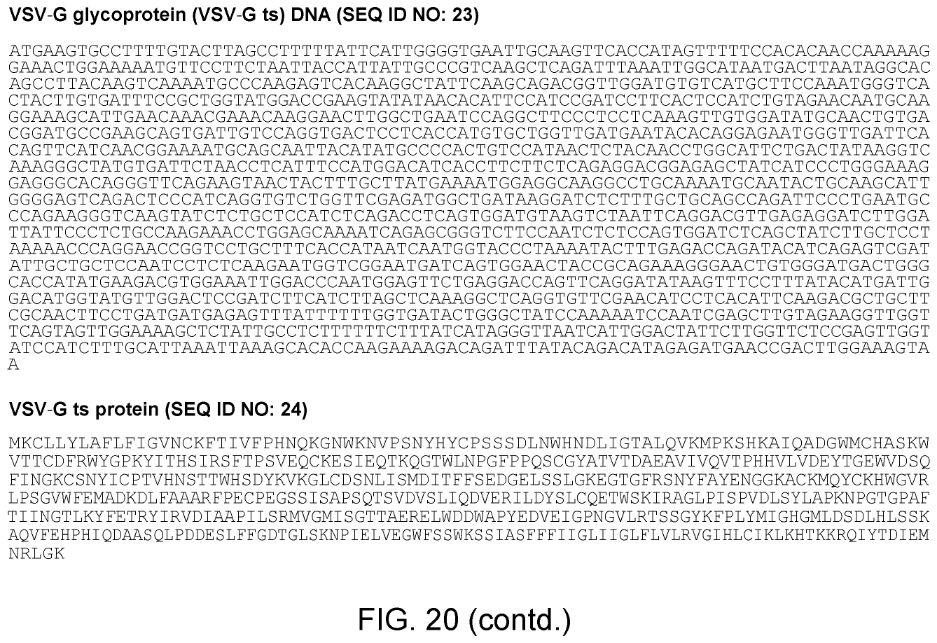

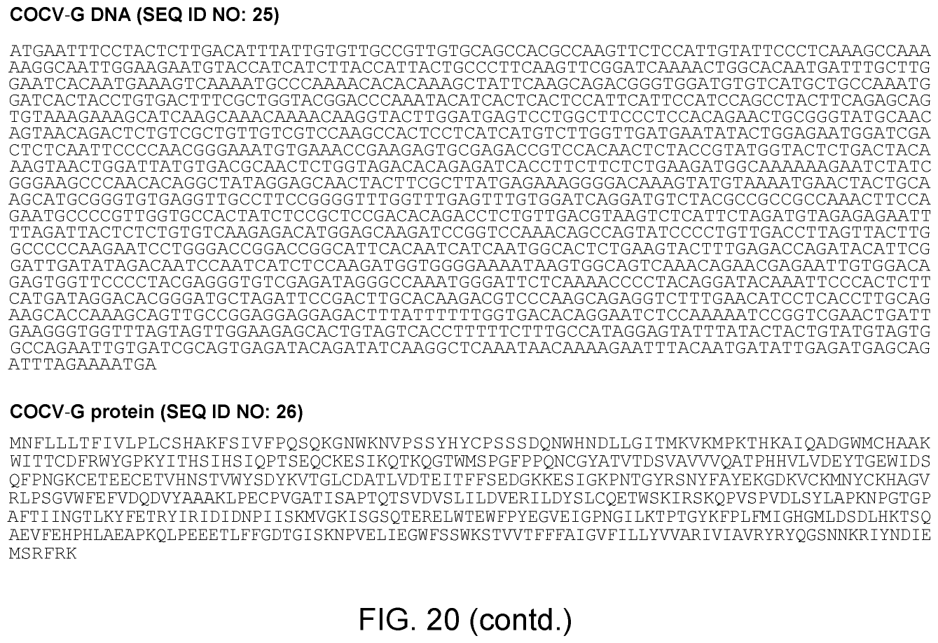

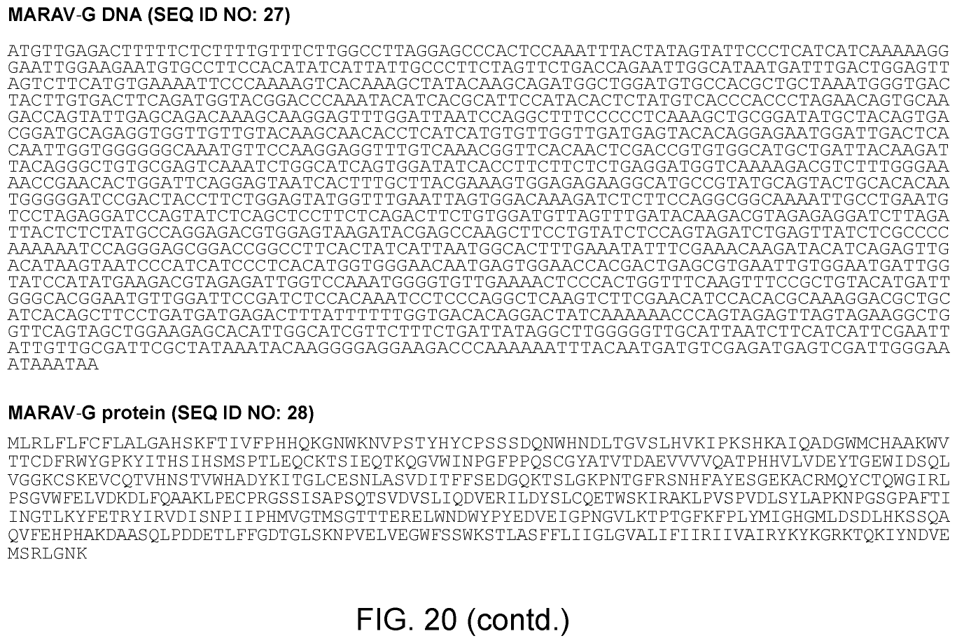

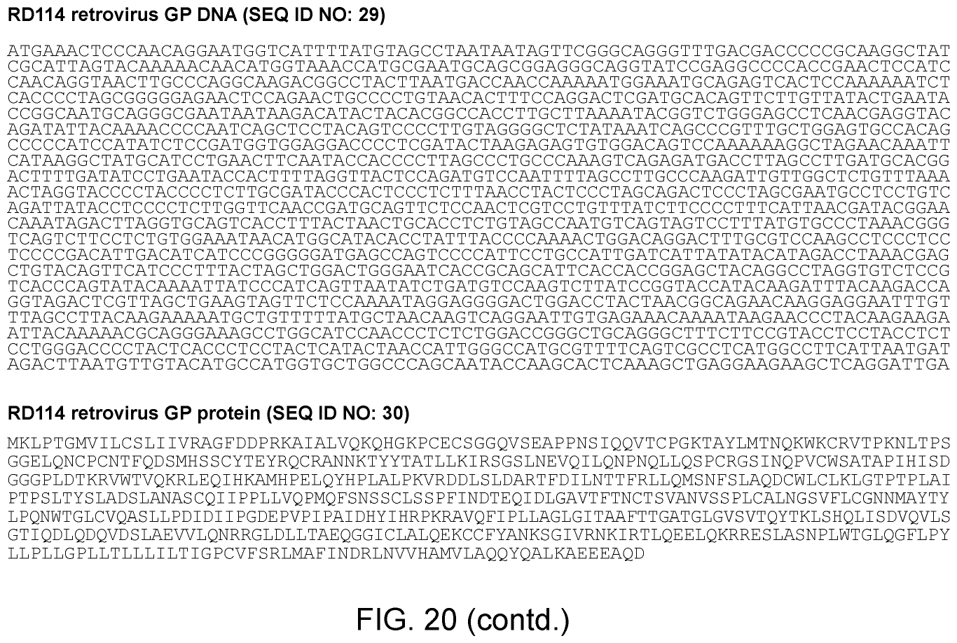

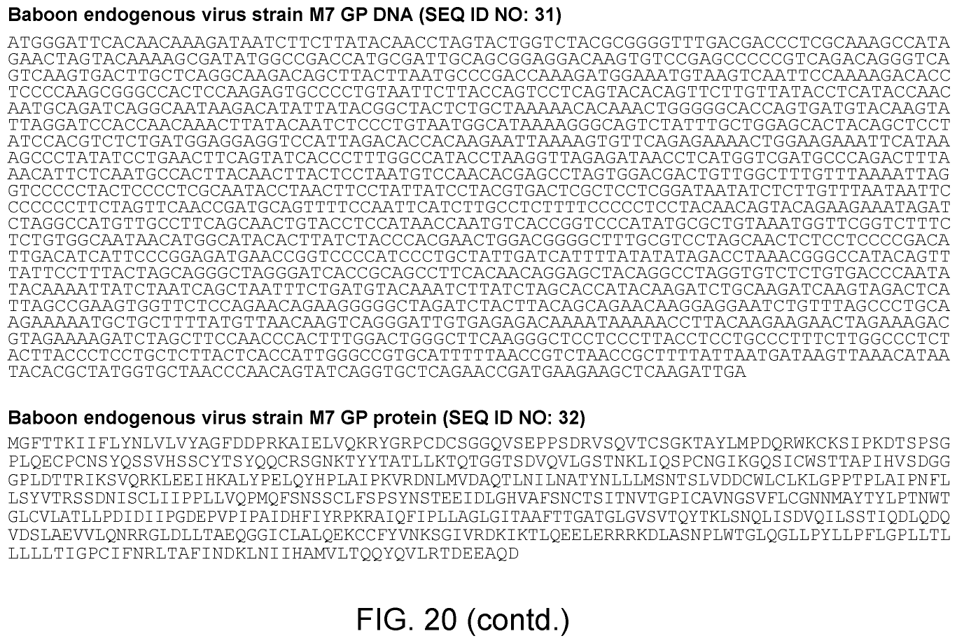

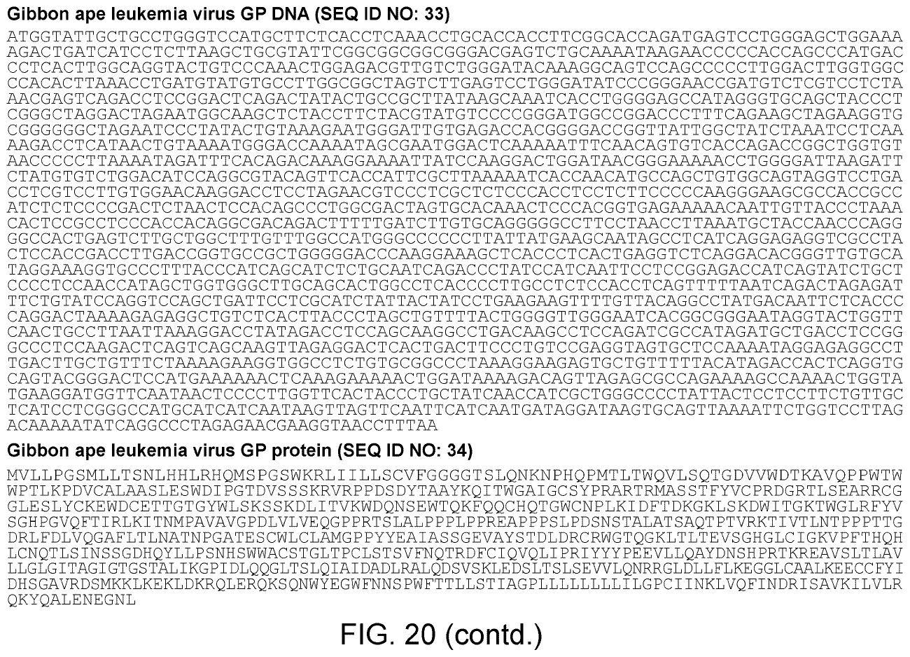

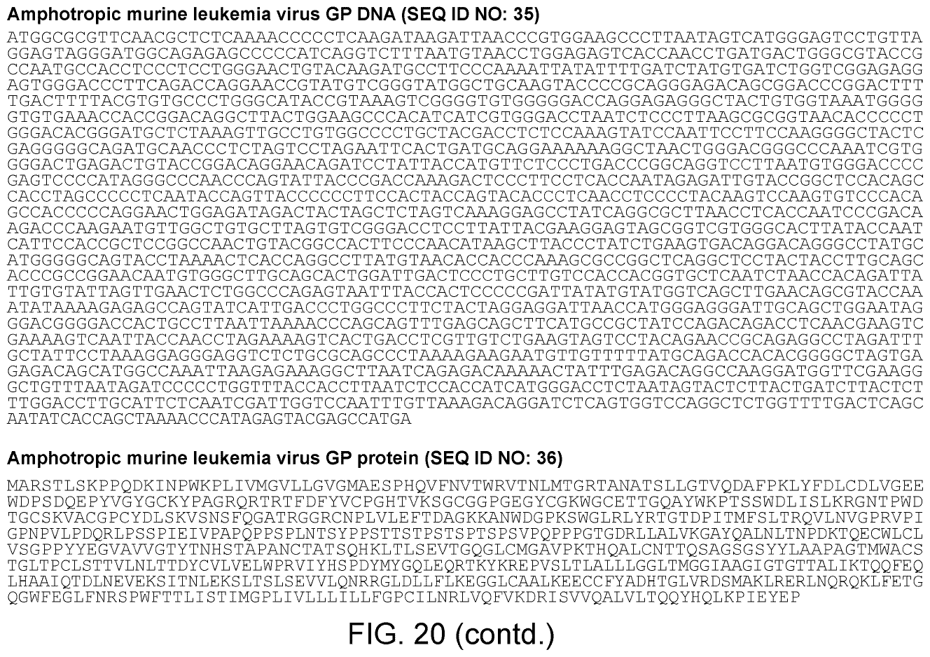

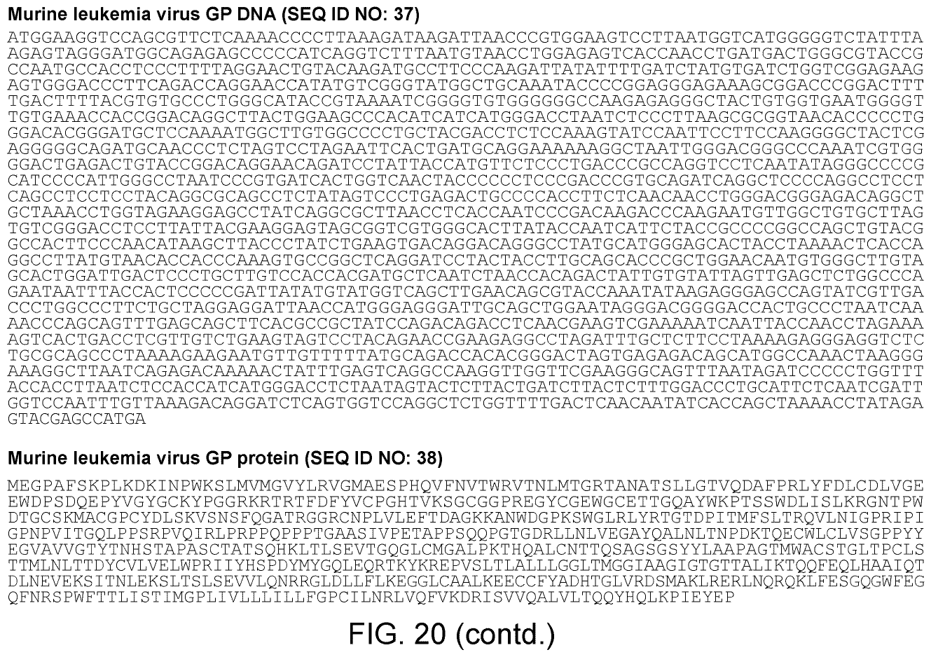

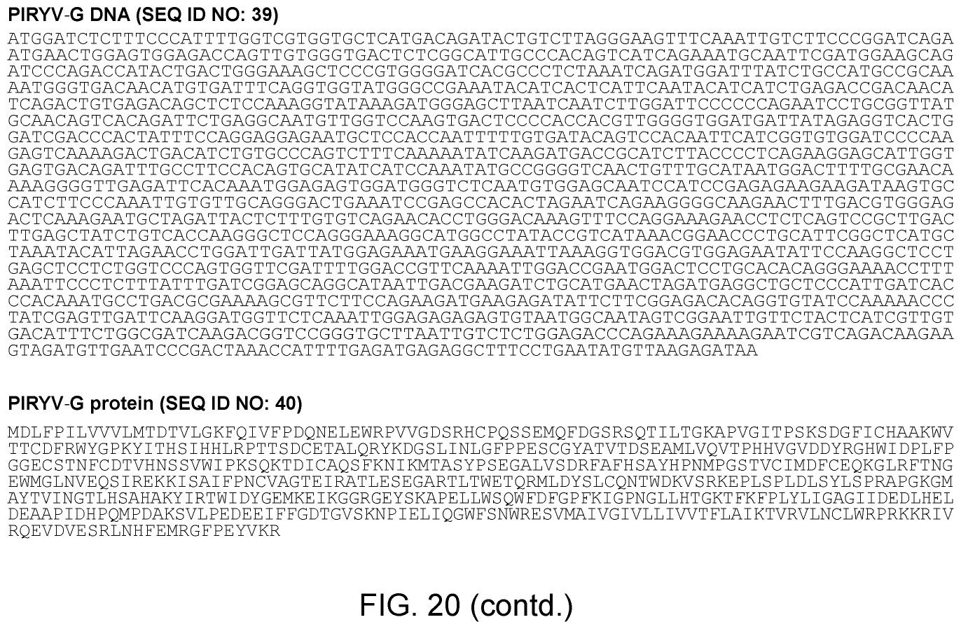

While the vestibular tissue was without any signs of degeneration, the outer hair cells in the section from the basal turn of the cochlea already appeared damaged at this time point despite LV-MYO7A treatment (FIG. 5D), and Annexin V staining indicated ongoing apoptosis throughout the supporting cell layer at 6 months of age (FIG. 5G). Counts of inner and outer cochlear hair cells showed LV-MYO7A treatment to decelerate hair cell degeneration, with close to 100% of expected hair cell numbers at 3 months of age and higher hair cell counts in the treated as compared to the non-treated contralateral ear at 6 months (FIG. 19). Comparing hair cell numbers at 3 and 6 months of age, however, treated cochleae also showed a progressive decline in hair cell survival, even though at slower kinetics as compared to the untreated ear, suggesting that supplementation of native myosin-VIIa does not fully rescue auditory hair cells in the long term.

To investigate the functional effects of LV-MYO7A treatment, hearing was determined at P90 through ABR measurement and compared to age-matched untreated Shaker-1^{mut} mice. None of the latter had measurable ABR thresholds. In contrast, hearing thresholds showed a trend for recovery of auditory function in the LV-MYO7A-treated cohort at all four tested frequencies (FIG. 5A). With a 91.6% success rate, 11 out of 12 treated animals showed some improvement in ABR thresholds and, thus, a functional effect of the LV-MYO7A gene therapy. The maximum effect was observed at a frequency of 8 kHz, with hearing thresholds as low as 40 dB in the best-responding mouse and a statistically significant improvement across the cohort. The mean improvement in hearing thresholds in the treated group was 15 dB at the high frequencies (16 and 32 kHz), 20 dB at the lowest frequency of 4 kHz, and 25 dB at 8 kHz. Despite this improvement, LV-MYO7A-treated mice did, however, not reach the normal hearing thresholds seen in control Shaker-1^{WT} mice.

A profound therapeutic effect was also achieved in terms of balance function measured at P60 (rotarod) and P90 (rotarod, force-plate actimetry). Rotarod testing demonstrated a halt in balance loss in LV-MYO7A-treated Shaker-1^{mut} animals upon treatment, as dysfunction did not progress to the degree seen in untreated Shaker-1^{mut} mice (FIG. 5B). Rather, rotarod times in LV-MYO7A-treated animals were preserved at an intermediate level between P90 Shaker-1^{het} mice, which have normal balance, and untreated P90 Shaker-1^{mut} mice. Differences between treated and untreated Shaker-1^{mut} animals were statistically significant at P60 and at P90. There was no statistical difference between the P60 and P90 time points within the group of LV-MYO7A-treated Shaker-1^{mut} animals, indicative of a long-term effect of the treatment, without any worsening of the phenotype since therapy.

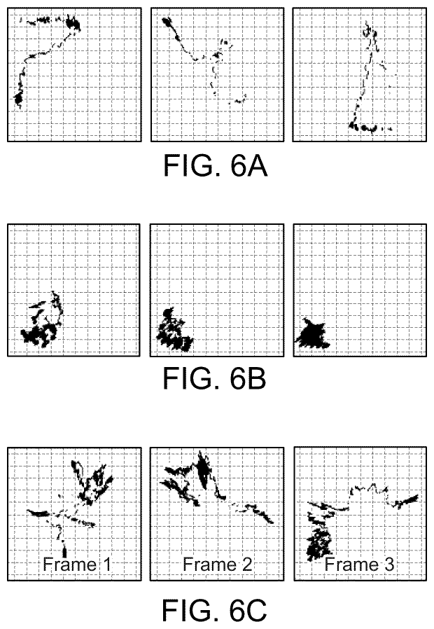

Actimeter tracking demonstrated more directed movement despite occasional circling for LV-MYO7A-treated Shaker-1^{mut} mice, while untreated Shaker-1^{mut} mice showed uncontrolled circling (FIGS. 5C and 6). Quantification of actimeter performance at P90 revealed no significant differences in the total distance traveled between control Shaker-1^{het}, untreated Shaker-1^{mut} and LV-MYO7A-treated Shaker-1^{mut} littermates, indicating that the mice in all groups were active (FIG. 5C). Importantly, however, there was a statistically significant reduction in the total area covered in LV-MYO7A-treated as compared to untreated Shaker-1^{mut} mice, and the performance of the treated mice was statistically similar to control Shaker-1^{het} littermates. Moreover, evaluation of the total degrees circled showed a reduction of circling behavior in the LV-MYO7A-treated as compared to age-matched untreated Shaker-1^{mut} mice (FIGS. 5C and 6). Interestingly, the degree of both left and right turns was reduced, although only the left ear had been treated with LV-MYO7A. Importantly, all mice (8 of 8 animals) in the LV-MYO7A-treated cohort showed this strong improvement in balance function, demonstrating a 100% success rate. Altogether, LV-MYO7A gene therapy at P16 temporarily improved hair cell survival and partially improved hearing function, but halted the progression of balance loss in homozygous Shaker-1^{mut} mice.

### Example 7: LV-MYO7A Gene Therapy at P4 and Delayed Treatment Improves Balance in Homozygous Shaker-1^{mut} Mice

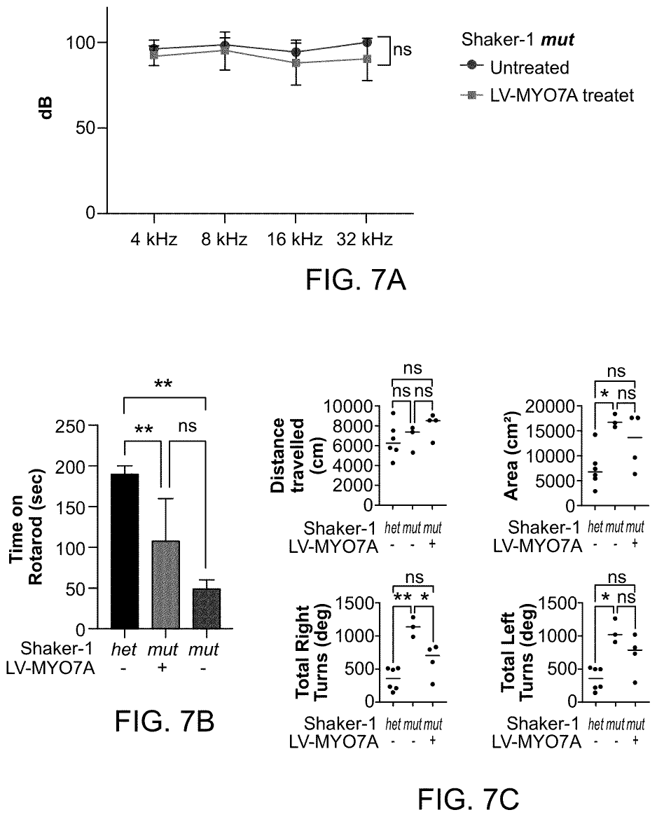

LV-MYO7A gene therapy at P16 showed a therapeutic effect in homozygous mutant Shaker-1^{mut} mice. However, the rescue was incomplete with hearing and balance not reaching wild-type levels. Therefore, we tested if delivery of MYO7A prior to maturation of hearing would improve rescue of function. For this, Shaker-1^{mut} mice were treated at P4 with VSV-G pseudotyped LV-MYO7A via canalostomy using the same vector batch and dose as before (1 μL). Functional effects were analyzed at P30 (hearing) and P90 (balance). ABR analysis to compare treated with untreated Shaker-1^{mut} littermates showed improvements of up to 20 dB in individual frequencies in 37% (3 out of 8 animals) of the treated mice (FIG. 7A). However, when mean hearing thresholds were averaged across the entire cohort of treated mice, no statistically significant improvement of hearing was found. Rotarod testing showed better performance of LV-MYO7A-treated as compared to untreated Shaker-1^{mut} animals, but the difference was not statistically significant (FIG. 7B). However, similar to P16 treated animals, P4 treated Shaker-1^{mut} mice showed a statistically significant reduction in circling behavior, as indicated by a reduced number of total degrees turned right or left on the actimeter plate (FIG. 7C). Of note, the treatment achieved a 100% success rate (4 of 4 treated animals) in terms of balance function in Shaker-1^{mut} mice. Altogether, treatment of homozygous Shaker-1^{mut} mice with LV-MYO7A at P4 did not increase the magnitude of therapeutic effects as compared to the gene therapy performed at P16.

As shown in FIG. 15, reversal of balance defects was also observed when Shaker-1^{mut} mice received the LV-MYO7A gene therapy at a significantly delayed timepoint (e.g., at age 2 months).

### Example 8: LV-MYO7A Gene Therapy at P4 Prevents Hearing Loss in Heterozygous Shaker-1^{het} Mice

As characterized above, heterozygous mutant Shaker-1^{het} mice harboring a single defective Myo7a allele develop significant hearing loss by 6 months of age. To determine whether LV-MYO7A gene therapy can halt or even rescue this late-onset hearing loss, Shaker-1^{het} mice were injected at P4 with 1 μL of VSV-G pseudotyped LV-MYO7A via canalostomy. As Shaker-1^{het} mice have normal balance function, the analysis of therapeutic effects of the gene transfer was restricted to the effects on hearing.

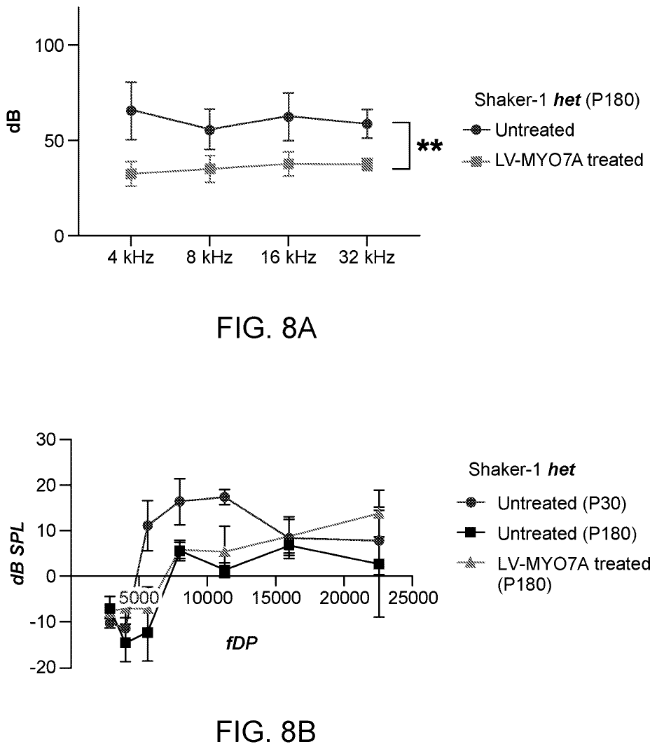

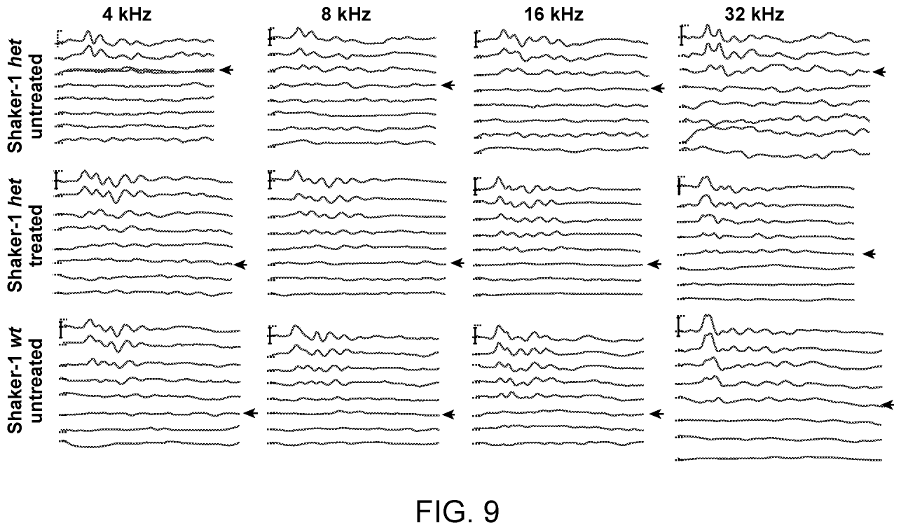

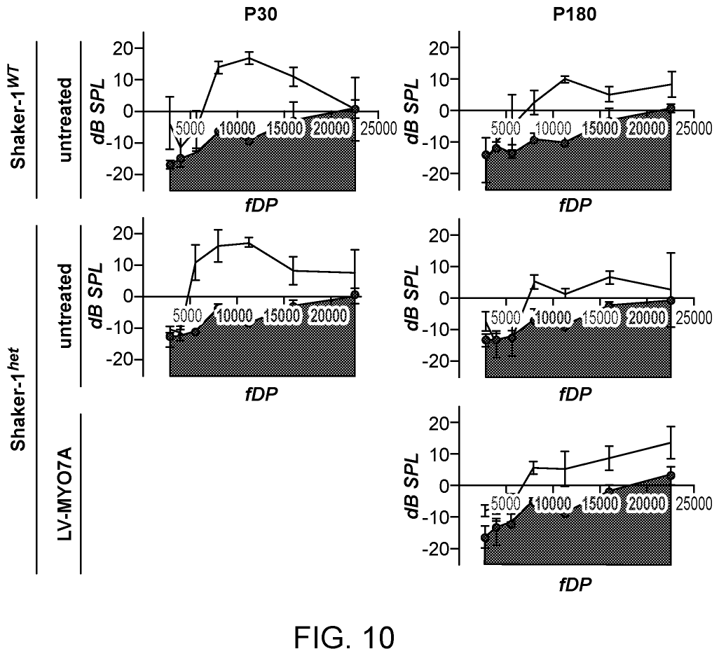

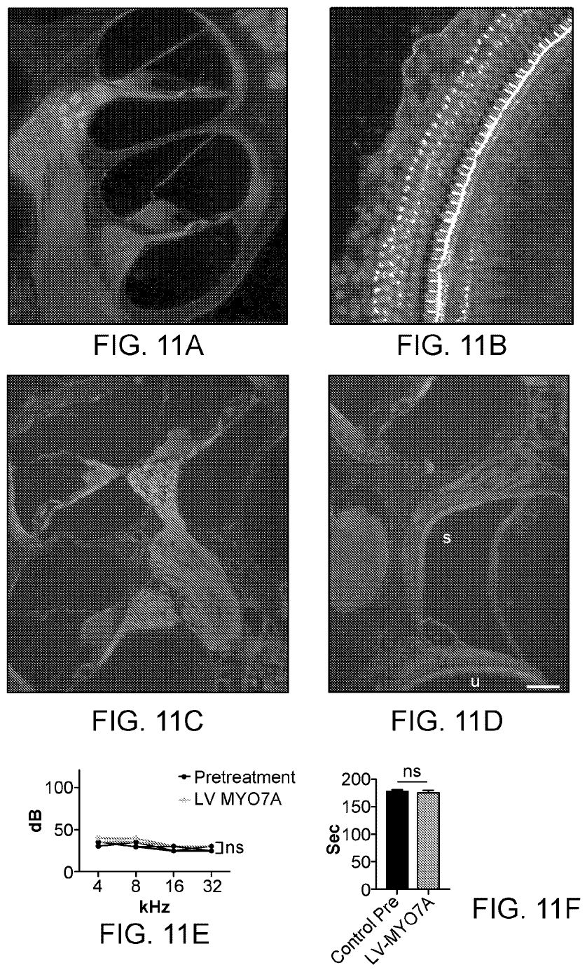

LV-MYO7A treatment at P4 prevented onset and progression of hearing loss at all frequencies. At 6 months of age (P180), all (N=8) LV-MYO7A-treated Shaker-1^{het} mice had maintained normal ABR thresholds and waveforms with no difference to Shaker-1^{WT} mice (FIGS. 8A and 9), showing that the gene therapeutic approach could completely rescue the phenotype in these animals at a 100% success rate.

In addition to ABR analysis, distortion product otoacoustic emission (DPOAE) thresholds were determined to characterize outer hair cell function. Shaker-1^{WT} mice and Shaker-1^{het} mice demonstrated normal outer hair cell function at P30 (FIG. 8B). In contrast, DPOAE thresholds in untreated Shaker-1^{het} mice were reduced at 6 months of age, in line with the late-onset hearing loss phenotype.

LV-MYO7A treated mice showed a decline in DPOAE that was similar to untreated Shaker-1^{het} mice at 6 months of age. However, Shaker-1^{WT} control mice also presented with decreased DPOAE thresholds at this age. Importantly, these were not significantly different from the LV-MYO7A treated Shaker-1^{het} mice. Also, despite the decline, DPOAE were still present in all groups tested, and there was mainly a reduction of DPOAE amplitude in the middle frequencies when comparing P30 to P180. This general and probably age-related decline in DPOAE performance that seems to be associated with the Shaker-1 mouse strain precludes evaluation of long-term functional outer hair cell rescue through LV-MYO7A. Altogether, LV-MYO7A completely prevented hearing loss in Shaker-1^{het} mice as indicated by normal ABR thresholds.

### Sequences of LV Components

| SEQ ID | | |
|---|---|---|
| NO | Sequence | Description |
| 2 | AGCTAGCTGCAGTAACGCCATTTTGCAAGGCATGGAAAAAT | SFFV |
| | ACCAAACCAAGAATAGAGAAGTTCAGATCAAGGGGGGTAC | promoter |
| | ATGAAAATAGCTAACGTTGGGCCAAACAGGATATCTGCGGT | |
| | GAGCAGTTTCGGCCCCGGCCCGGGGCCAAGAACAGATGGTC | |
| | ACCGCAGTTTCGGCCCCGGCCCGAGGCCAAGAACAGATGGT | |
| | CCCCAGATATGGCCCAACCCTCAGCAGTTTCTTAAGACCCAT | |
| | CAGATGTTTCCAGGCTCCCCCAAGGACCTGAAATGACCCTGC | |
| | GCCTTATTTGAATTAACCAATCAGCCTGCTTCTCGCTTCTGTT | |
| | CGCGCGCTTCTGCTTCCCGAGCTCTATAAAAGAGCTCACAAC | |
| | CCCTCACTCGGCGCGCCAGTCCTCCGA | |
| 3 | TTAATGTAGTCTTATGCAATACTCTTGTAGTCTTGCAACATG | RSV |
| | GTAACGATGAGTTAGCAACATGCCTTACAAGGAGAGAAAAA | promoter |
| | GCACCGTGCATGCCGATTGGTGGAAGTAAGGTGGTACGATC | |
| | GTGCCTTATTAGGAAGGCAACAGACGGGTCTGACATGGATT | |
| | GGACGAACCACTGAATTGCCGCATTGCAGAGATATTGTATTT | |
| | AAGTGCCTAGCTCGATACAATAAAC | |
| 4 | GGTCTCTCTGGTTAGACCAGATCTGAGCCTGGGAGCTCTCTG | R-U5 region |
| | GCTAACTAGGGAACCCACTGCTTAAGCCTCAATAAAGCTTGC | |
| | CTTGAGTGCTTCAAGTAGTGTGTGCCCGTCTGTTGTGTGACT | |
| | CTGGTAACTAGAGATCCCTCAGACCCTTTTAGTCAGTGTGGA | |
| | AAATCTCTAGCAG | |
| 5 | CTGGTGAGT | Splice donor |
| | | (SD) |
| 6 | TGAGTACGCCAAAAATTTTGACTAGCGGAGGCTAGAAGGAG | Psi |
| | AGAG | packaging |
| | | signal |
| 7 | GGAGCTTTGTTCCTTGGGTTCTTGGGAGCAGCAGGAAGCACT | Rev- |
| | ATGGGCGCAGCCTCAATGACGCTGACGGTACAGGCCAGACA | response |
| | ATTATTGTCTGGTATAGTGCAGCAGCAGAACAATTTGCTGAG | element |
| | GGCTATTGAGGCGCAACAGCATCTGTTGCAACTCACAGTCTG | |
| | GGGCATCAAGCAGCTCCAGGCAAGAATCCTGGCTGTGGAAA | |
| | GATACCTAAAGGATCAACAGCTCCTGGGGATTTG | |
| 8 | AACTTTTAAAAGAAAAGGGGGGATTGGGGGGTACAGTGCAG | Polypurine |
| | GGGAAAGAATAGTAGACATAATAGCAACAGACATACAAACT | tract |
| | AAAGAATTACAAAAACAAATTACAAAAATTCAAAATTTTAT | |
| | C | |
| 9 | TAGTGAATAGAGTTAGGCAGGGA | Splice |
| | | acceptor |
| | | (SA) |
| 10 | TCGACAATCAACCTCTGGATTACAAAATTTGTGAAAGATTGA | Post- |
| | CTGGTATTCTTAACTATGTTGCTCCTTTTACGCTATGTGGATA | transcriptional |
| | CGCTGCTTTAATGCCTTTGTATCATGCTATTGCTTCCCGTATG | Regulatory |
| | GCTTTCATTTTCTCCTCCTTGTATAAATCCTGGTTGCTGTCTC | Element |
| | TTTATGAGGAGTTGTGGCCCGTTGTCAGGCAACGTGGCGTGG | (PRE) |
| | TGTGCACTGTGTTTGCTGACGCAACCCCCACTGGTTGGGGCA | |
| | TTGCCACCACCTGTCAGCTCCTTTCCGGGACTTTCGCTTTCCC | |
| | CCTCCCTATTGCCACGGCGGAACTCATCGCCGCCTGCCTTGC | |
| | CCGCTGCTGGACAGGGGCTCGGCTGTTGGGCACTGACAATTC | |
| | CGTGGTGTTGTCGGGGAAGCTGACGTCCTTTCCATGGCTGCT | |
| | CGCCTGTGTTGCCACCTGGATTCTGCGCGGGACGTCCTTCTG | |
| | CTACGTCCCTTCGGCCCTCAATCCAGCGGACCTTCCTTCCCG | |
| | CGGCCTGCTGCCGGCTCTGCGGCCTCTTCCGCGTCTTCGCCTT | |
| | CGCCCTCAGACGAGTCGGATCTCCCTTTGGGCCGCCTCCCCG | |
| | CCTGGA | |
| 11 | TGGAAGGGCTAATTCACTCCCAACGAAGACAAGATCTGCTTT | ΔU3-R |
| | TTGCTTGTACTGGGTCTCTCTGGTTAGACCAGATCTGAGCCT | |
| | GGGAGCTCTCTGGCTAACTAGGGAACCCACTGCTTAAGCCTC | |
| | AATAAAGCTTGCCTTGAGTGCTTC | |
| 12 | GGTCTCTCTGGTTAGACCAGATCTGAGCCTGGGAGCTCTCTG | Repeat |
| | GCTAACTAGGGAACCCACTGCTTAAGCCTCAATAAAGCTTGC | region (R) |
| | CTTGAGTGCTTC | |
| 13 | TGGCGCCCGAACAGGGAC | Primer |
| | | binding site |
| | | (PBS) |
| 14 | GATGGGTGCGAGAGCGTCAGTATTAAGCGGGGGAGAATTAG | ΔGAG |
| | ATCGCGATGGGAAAAAATTCGGTTAAGGCCAGGGGGAAAGA | |
| | AAAAATATAAATTAAAACATATAGTATGGGCAAGCAGGGAG | |
| | CTAGAACGATTCGCAGTTAATCCTGGCCTGTTAGAAACATCA | |
| | GAAGGCTGTAGACAAATACTGGGACAGCTACAACCATCCCT | |
| | TCAGACAGGATCAGAAGAACTTAGATCATTATATAATACAG | |
| | TAGCAACCCTCTATTGTGTGCATCAAAGGATAGAGATAAAA | |
| | GACACCAAGGAAGCTTTAGACAAGATAGAGGAAGAGCAAA | |
| | ACAAAAGTAAGACCACCGCACAGCAAGCGGCCGCTGAT | |
| 15 | GCCCCTCTCCCTCCCCCCCCCCTAACGTTACTGGCCGAAGCC | IRES |
| | GCTTGGAATAAGGCCGGTGTGCGTTTGTCTATATGTTATTTTC | |
| | CACCATATTGCCGTCTTTTGGCAATGTGAGGGCCCGGAAACC | |
| | TGGCCCTGTCTTCTTGACGAGCATTCCTAGGGGTCTTTCCCCT | |
| | CTCGCCAAAGGAATGCAAGGTCTGTTGAATGTCGTGAAGGA | |
| | AGCAGTTCCTCTGGAAGCTTCTTGAAGACAAACAACGTCTGT | |
| | AGCGACCCTTTGCAGGCAGCGGAACCCCCCACCTGGCGACA | |
| | GGTGCCTCTGCGGCCAAAAGCCACGTGTATAAGATACACCT | |
| | GCAAAGGCGGCACAACCCCAGTGCCACGTTGTGAGTTGGAT | |
| | AGTTGTGGAAAGAGTCAAATGGCTCTCCTCAAGCGTATTCAA | |
| | CAAGGGGCTGAAGGATGCCCAGAAGGTACCCCATTGTATGG | |
| | GATCTGATCTGGGGCCTCGGTGCACATGCTTTACATGTGTTT | |
| | AGTCGAGGTTAAAAAAACGTCTAGGCCCCCCGAACCACGGG | |
| | GACGTGGTTTTCCTTTGAAAAACACGATGATAATATGGCCAC | |
| | AACC | |
| 18 | ACATAACTTACGGTAAATGGCCCGCCTGGCTGACCGCCCAAC | cytomegalo |
| | GACCCCCGCCCATTGACGTCAATAATGACGTATGTTCCCATA | virus |
| | GTAACGCCAATAGGGACTTTCCATTGACGTCAATGGGTGGA | enhancer- |
| | GTATTTACGGTAAACTGCCCACTTGGCAGTACATCAAGTGTA | chicken |
| | TCATATGCCAAGTACGCCCCCTATTGACGTCAATGACGGTAA | beta-actin |
| | ATGGCCCGCCTGGCATTATGCCCAGTACATGACCTTATGGGA | promoter |
| | CTTTCCTACTTGGCAGTACATCTACGTATTAGTCATC | (CAG) |
| 41 | AAAAGAAAAGGGGGGACTGGAA | U3 PPT |

### REFERENCES

1. Kros, C. J., Marcotti, W., van Netten, S. M., Self, T. J., Libby, R. T., Brown, S. D., Richardson, G. P., and Steel, K. P. (2002). Reduced climbing and increased slipping adaptation in cochlear hair cells of mice with Myo7a mutations. Nat Neurosci 5, 41-47.
2. Li, S., Mecca, A., Kim, J., Caprara, G. A., Wagner, E. L., Du, T. T., Petrov, L., Xu, W., Cui, R., Rebustini, I. T., Kachar, B., et al. (2020). Myosin-VIIa is expressed in multiple isoforms and essential for tensioning the hair cell mechanotransduction complex. Nat Commun 11, 2066.
3. Weil, D., Kussel, P., Blanchard, S., Levy, G., Levi-Acobas, F., Drira, M., Ayadi, H., and Petit, C. (1997). The autosomal recessive isolated deafness, DFNB2, and the Usher 1B syndrome are allelic defects of the myosin-VIIA gene. Nat Genet 16, 191-193.
4. Géléoc, G.G.S., and El-Amraoui, A. (2020). Disease mechanisms and gene therapy for Usher syndrome. Hear Res 394, 107932.
5. Testa, F., Melillo, P., Bonnet, C., Marcelli, V., de Benedictis, A., Colucci, R., Gallo, B., Kurtenbach, A., Rossi, S., Marciano, E., Auricchio, A., et al. (2017). Clinical Presentation and Disease Course of Usher Syndrome Because of Mutations in Myo7a or Ush2a. Retina 37, 1581-1590.
6. Koenekoop, R. K., Arriaga, M. A., Trzupek, K. M., and Lentz, J. J. (1999). Usher Syndrome Type I. In GeneReviews® [Internet], M. P. Adam, G. M. Mirzaa, and R. A. Pagon, eds. (Seattle (WA): University of Washington, Seattle).
7 Tamagawa, Y., Kitamura, K., Ishida, T., Ishikawa, K., Tanaka, H., Tsuji, S., and Nishizawa, M. (1996). A gene for a dominant form of non-syndromic sensorineural deafness (DFNA11) maps within the region containing the DFNB2 recessive deafness gene. Hum Mol Genet 5, 849-852.
8. Gibson, F., Walsh, J., Mburu, P., Varela, A., Brown, K. A., Antonio, M., Beisel, K. W., Steel, K. P., and Brown, S. D. (1995). A type VII myosin encoded by the mouse deafness gene shaker-1. Nature 374, 62-64.
9. Xiong, A., Haithcock, J., Liu, Y., Eusner, L., McConnell, M., White, H. D., Belknap, B., and Forgacs, E. (2018). The shaker-1 mouse myosin VIIa deafness mutation results in a severely reduced rate of the ATP hydrolysis step. J Biol Chem 293, 819-829.
10. Lord, E. M., and Gates, W. H. (1929). Shaker, a New Mutation of the House Mouse (Mus musculus). Am. Nat. 63.
11. Shnerson, A., Lenoir, M., van de Water, T. R., and Pujol, R. (1983). The pattern of sensorineural degeneration in the cochlea of the deaf shaker-1 mouse: ultrastructural observations. Brain Res 285, 305-315.
12. Self, T., Mahony, M., Fleming, J., Walsh, J., Brown, S. D., and Steel, K. P. (1998). Shaker-1 mutations reveal roles for myosin VIIA in both development and function of cochlear hair cells. Development 125, 557-566.
13. Ivanchenko, M. V., Hanlon, K. S., Hathaway, D. M., Klein, A. J., Peters, C. W., Li, Y., Tamvakologos, P. I., Nammour, J., Maguire, C. A., and Corey, D. P. (2021). AAV-S: A versatile capsid variant for transduction of mouse and primate inner ear. Mol Ther 21, 382-398.
14. Taiber, S., Cohen, R., Yizhar-Barnea, O., Sprinzak, D., Holt, J. R., and Avraham, K. B. (2021). Neonatal AAV gene therapy rescues hearing in a mouse model of SYNE4 deafness. EMBO Mol Med 13, e13259.
15. Chamberlain, K., Riyad, J. M., and Weber, T. (2016). Expressing Transgenes That Exceed the Packaging Capacity of Adeno-Associated Virus Capsids. Hum Gene Ther Methods 27, 1-12.
16. Al-Moyed, H., Cepeda, A. P., Jung, S., Moser, T., Kugler, S., and Reisinger, E. (2019). A dual-AAV approach restores fast exocytosis and partially rescues auditory function in deaf otoferlin knock-out mice. EMBO Mol Med 11, e9396.
17. Reisinger, E. (2020). Dual-AAV delivery of large gene sequences to the inner ear. Hear Res 394, 107857.
18. Maass, J. C., Berndt, F. A., Canovas, J., and Kukuljan, M. (2013). p27Kipl knockdown induces proliferation in the organ of Corti in culture after efficient shRNA lentiviral transduction. J Assoc Res Otolaryngol 14, 495-508.
19. Wei, Y., Fu, Y., Liu, S., Xia, G., and Pan, S. (2013). Effect of lentiviruses carrying enhanced green fluorescent protein injected into the scala media through a cochleostomy in rats. Am J Otolaryngol 34, 301-307.
20. Pan, S., Wan, J., Liu, S., Zhang, S., Xiong, H., Zhou, J., Xiong, W., Yu, K., and Fu, Y. (2013). Lentivirus carrying the Atohl gene infects normal rat cochlea. Neural Regen Res 8, 1551-1559.
21. Han, J. J., Mhatre, A. N., Wareing, M., Pettis, R., Gao, W. Q., Zufferey, R. N., Trono, D., and Lalwani, A. K. (1999). Transgene expression in the guinea pig cochlea mediated by a lentivirus-derived gene transfer vector. Hum Gene Ther 10, 1867-1873.
22. Miyoshi, H., Blomer, U., Takahashi, M., Gage, F. H., and Verma, I. M. (1998). Development of a self-inactivating lentivirus vector. J Virol 72, 8150-8157.
23. Zufferey, R., Dull, T., Mandel, R. J., Bukovsky, A., Quiroz, D., Naldini, L., and Trono, D. (1998). Self-inactivating lentivirus vector for safe and efficient in vivo gene delivery. J Virol 72, 9873-9880.
24. Cesana, D., Ranzani, M., Volpin, M., Bartholomae, C., Duros, C., Artus, A., Merella, S., Benedicenti, F., Sergi Sergi, L., Sanvito, F., Brombin, C., et al. (2014). Uncovering and dissecting the genotoxicity of self-inactivating lentiviral vectors in vivo. Mol Ther 22, 774-785.
25. Schwarzer, A., Talbot, S. R., Selich, A., Morgan, M., Schott, J. W., Dittrich-Breiholz, O., Bastone, A. L., Weigel, B., Ha, T. C., Dziadek, V., Gijsbers, R., et al. (2021). Predicting genotoxicity of viral vectors for stem cell gene therapy using gene expression-based machine learning. Mol Ther 29, 3383-3397.
26. Tucci, F., Galimberti, S., Naldini, L., Valsecchi, M. G., and Aiuti, A. (2022). A systematic review and meta-analysis of gene therapy with hematopoietic stem and progenitor cells for monogenic disorders. Nat Commun 13, 1315.
27. Kalinec, G. M., Webster, P., Lim, D. J., and Kalinec, F. (2003). A cochlear cell line as an in vitro system for drug ototoxicity screening. Audiol Neurootol 8, 177-189.
28. Mizuguchi, H., Xu, Z., Ishii-Watabe, A., Uchida, E., and Hayakawa, T. (2000). IRES-dependent second gene expression is significantly lower than cap-dependent first gene expression in a bicistronic vector. Mol Ther 1, 376-382.
29. Shearer, A. E., Hildebrand, M. S., Schaefer, A. M., and Smith, R. J. H. (1999). Genetic Hearing Loss Overview. In GeneReviews® [Internet], M. P. Adam, G. M. Mirzaa, and R. A. Pagon, eds. (Seattle (WA): University of Washington, Seattle).
30. Sloan-Heggen, C. M., Bierer, A. O., Shearer, A. E., Kolbe, D. L., Nishimura, C. J., Frees, K. L., Ephraim, S. S., Shibata, S. B., Booth, K. T., Campbell, C. A., Ranum, P. T., et al. (2016). Comprehensive genetic testing in the clinical evaluation of 1119 patients with hearing loss. Hum Genet 135, 441-450.
31. Morgan, M., Schott, J. W., Rossi, A., Landgraf, C., Warnecke, A., Staecker, H., Lesinski-Schiedat, A., Schlegelberger, B., Büning, H., Auber, B., and Schambach, A. (2020). Gene therapy as a possible option to treat hereditary hearing loss. Med Genet 32, 149-159.
32. Büning, H., Schambach, A., Morgan, M., Rossi, A., Wichova, H., Staecker, H., Warnecke, A., and Lenarz, T. (2020). Challenges and advances in translating gene therapy for hearing disorders. Expert Rev Precis Med Drug Dev 5, 23-34.
33. Lustig, L., and Akil, O. (2018). Cochlear Gene Therapy. Cold Spring Harb Perspect Med 9, a033191.
34. Akil, O., Dyka, F., Calvet, C., Emptoz, A., Lahlou, G., Nouaille, S., Boutet de Monvel, J., Hardelin, J. P., Hauswirth, W. W., Avan, P., Petit, C., et al. (2019). Dual AAV-mediated gene therapy restores hearing in a DFNB9 mouse model. Proc Natl Acad Sci USA 116, 4496-4501.
35. Wagenaar, M., van Aarem, A., Huygen, P., Pieke-Dahl, S., Kimberling, W., and Cremers, C. (1999). Hearing impairment related to age in Usher syndrome types 1B and 2A. Arch Otolaryngol Head Neck Surg 125, 441-445.
36. Isgrig, K., Shteamer, J. W., Belyantseva, I. A., Drummond, M. C., Fitzgerald, T. S., Vijayakumar, S., Jones, S. M., Griffith, A. J., Friedman, T. B., Cunningham, L. L., and Chien, W. W. (2017). Gene Therapy Restores Balance and Auditory Functions in a Mouse Model of Usher Syndrome. Mol Ther 30, 975.
37. Geng, R., Omar, A., Gopal, S. R., Chen, D. H., Stepanyan, R., Basch, M. L., Dinculescu, A., Furness, D. N., Saperstein, D., Hauswirth, W., Lustig, L. R., et al. (2017). Modeling and Preventing Progressive Hearing Loss in Usher Syndrome III. Sci Rep 7, 13480.
38. Boucher, S., Tai, F. W. J., Delmaghani, S., Lelli, A., Singh-Estivalet, A., Dupont, T., Niasme-Grare, M., Michel, V., Wolff, N., Bahloul, A., Bouyacoub, Y., et al. (2020). Ultrarare heterozygous pathogenic variants of genes causing dominant forms of early-onset deafness underlie severe presbycusis. Proc Natl Acad Sci USA 117, 31278-31289.
39. Dull, T., Zufferey, R., Kelly, M., Mandel, R. J., Nguyen, M., Trono, D., and Naldini, L. (1998). A third-generation lentivirus vector with a conditional packaging system. J Virol 72, 8463-8471.
40. Schlecker, C., Praetorius, M., Brough, D. E., Presler, R. G., Jr., Hsu, C., Plinkert, P. K., and Staecker, H. (2011). Selective atonal gene delivery improves balance function in a mouse model of vestibular disease. Gene Ther 18, 884-890.
41. Isgrig, K., and Chien, W. W. (2018). Posterior Semicircular Canal Approach for Inner Ear Gene Delivery in Neonatal Mouse. J Vis Exp 133, 56648.
42. Risoud, M., Sircoglou, J., Dedieu, G., Tardivel, M., Vincent, C., and Bonne, N. X. (2017). Imaging and cell count in cleared intact cochlea in the Mongolian gerbil using laser scanning confocal microscopy. Eur Ann Otorhinolaryngol Head Neck Dis 134, 221-224.

1. A method for treating or preventing Usher 1B syndrome in a patient in need thereof comprising administering to the patient an effective amount of a composition comprising a self-inactivating (SIN) lentiviral expression vector pseudotyped with a viral envelope glycoprotein that is configured to bind to a receptor expressed in an inner ear cell, wherein the SIN lentiviral expression vector comprises:
a) a 5′ long terminal repeat (LTR) region comprising or consisting of a constitutively active or inducible heterologous promoter sequence and the Repeat (R)-U5 sequence of SEQ ID NO: 4, wherein the constitutively active or inducible heterologous promoter sequence is located upstream of the R-U5 sequence;
b) a 5′ untranslated region (UTR) comprising the splice donor (SD) sequence of SEQ ID NO: 5, a packaging signal sequence, a Rev-responsive element (RRE), the splice acceptor (SA) sequence of SEQ ID NO: 9, and optionally, a polypurine tract (PPT) region;
c) an internal promoter operably linked to a cargo sequence, wherein the cargo sequence comprises a gene sequence encoding MyoVIIa isoform 1, and optionally a gene sequence encoding a reporter protein;
d) RNA processing elements comprising a posttranscriptional regulatory element (PRE), wherein the PRE is located downstream of the cargo sequence, and
e) a 3′ LTR region comprising or consisting of the deleted U3-R sequence of SEQ ID NO: 11, wherein the 3′ LTR region is located downstream of the PRE, and
wherein the SIN lentiviral expression vector does not comprise a polypurine tract (PPT) region between the cargo sequence and the PRE.

2. The method of claim 1, wherein the SIN lentiviral expression vector lacks vif, vpr, vpu, nef, and optionally tat genes and/or wherein the SIN lentiviral expression vector comprises a PPT region of SEQ ID NO: 41 between the PRE and the 3′ LTR region.

3. The method of claim 1, wherein the gene sequence encoding MyoVIIa isoform 1 comprises SEQ ID NO: 1; or
wherein the internal promoter is a SFFV promoter comprising SEQ ID NO: 2 or a CAG promoter comprising SEQ ID NO: 18; or
wherein the constitutively active or inducible heterologous promoter sequence is a Rous sarcoma virus (RSV) promoter sequence, a human cytomegalovirus (CMV) promoter sequence, an HIV promoter sequence, a eukaryotic promoter sequence, or a Tet-regulated promoter sequence, optionally wherein the RSV promoter sequence comprises SEQ ID NO: 3; or
wherein the packaging signal sequence comprises SEQ ID NO: 6 and/or the RRE comprises SEQ ID NO: 7; or
wherein the PPT region of the 5′ UTR comprises SEQ ID NO: 8 and/or is located downstream of the RRE; or
wherein the PRE comprises SEQ ID NO: 10.

4. (canceled)

5. (canceled)

6. (canceled)

7. (canceled)

8. (canceled)

9. The method of claim 1, wherein the 5′ UTR comprises a primer binding site (PBS), optionally wherein the PBS comprises the sequence of SEQ ID NO: 13.

10. The method of claim 1, wherein the 5′ UTR comprises a mutant group-specific antigen (Gag) sequence, optionally wherein the mutant Gag sequence comprises SEQ ID NO: 14.

11. The method of claim 1, wherein the cargo sequence further comprises an internal ribosomal entry site (IRES) that is interspersed between the gene sequence encoding MyoVIIa isoform 1, and the gene sequence encoding a reporter protein, optionally wherein the IRES comprises SEQ ID NO: 15.

12. The method of claim 1, wherein the reporter protein is selected from the group consisting of dTomato, TagBFP, Azurite, EBFP2, mKalama1, Sirius, Sapphire, T-Sapphire, ECFP, Cerulean, SCFP3A, mTurquoise, monomeric Midoriishi-Cyan, TagCFP, mTFP1, EGFP, Emerald, Superfolder GFP, Monomeric Azami Green, TagGFP2, mUKG, mWasabi, EYFP, Citrine, Venus, SYFP2, TagYFP, Monomeric Kusabira-Orange, mKOK, mKO2, mOrange, mOrange2, mRaspberry, mCherry, dsRed, mStrawberry, mTangerine, tdTomato, TagRFP, TagRFP-T, mApple, mRuby, mPlum, HcRed-Tandem, mKate2, mNeptune, NirFP, TagRFP657, IFP1.4, iRFP, mKeima Red, LSS-mKatel, LSS-mKate2, PA-GFP, PAmCherryl, PATagRFP, Kaede (green), Kaede (red), KikGR1 (green), KikGR1 (red), PS-CFP2, PS-CFP2, mEos2 (green), mEos2 (red), PSmOrange, and Dronpa.

13. The method of claim 1, wherein the SIN lentiviral expression vector is derived from a lentivirus selected from the group consisting of human immunodeficiency virus (e.g., HIV-1, HIV-2), simian immunodeficiency virus (SIV), feline immunodeficiency virus (FIV), and equine infectious anemia virus (EIAV).

14. The method of claim 1, wherein the patient is heterozygous or homozygous for a MYO7A genetic mutation.

15. The method of claim 1, wherein the patient has been diagnosed with Usher syndrome type IB.

16. The method of claim 1, wherein the SIN lentiviral expression vector is pseudotyped with a viral envelope glycoprotein capable of binding to a receptor selected from the group consisting of the LDL-receptor and LDL-R family members, the SLC1 A5-receptor, the Pit1/2-receptor and the PIRYV-G-receptor; or wherein the SIN lentiviral expression vector is pseudotyped with a viral envelope glycoprotein capable of binding to the LDL-receptor, wherein the viral envelope glycoprotein is selected from the group comprising MARAV-G, COCV-G, VSV-G and VSV-G ts.

17. (canceled)

18. The method of claim 1, wherein the inner ear cell is selected from the group consisting of cells of the organ of Corti including supporting cells as well as inner and outer hair cells, spiral ganglion neurons and glial cells, cells of the stria vascularis and spiral ligament, lateral wall fibrocytes, type I and II vestibular hair cells, vestibular supporting cells and vestibular ganglion neurons.

19. The method of claim 1, wherein the composition is administered via a cochlea implant route, round window injection, oval window injection, canalostomy, cochleostomy or injection into the endolymphatic sac.

20. The method of claim 1, wherein the composition has a titer of at least 7.78×10⁶ TU/mL.

21. The method of claim 20, wherein the titer of the composition is concentrated using ultracentifugation, microfiltration, ultrafiltration, chromatography, density gradient ultracentrifugation, tangential flow microfiltration, or precipitation.

22. The method of claim 1, further comprising administering to the patient a second vector comprising gag and pol, and optionally tat, a third plasmid comprising the viral envelope glycoprotein, and a fourth vector comprising regulatory gene Rev.

23. The method of claim 1, wherein administration of the composition ameliorates presbycusis or balance dysfuntion in the patient.

24. The method of claim 1, further comprising separately, sequentially or simultaneously administering one or more additional therapeutic agents to the patient.

25. The method of claim 1, wherein the composition is administered to the patient daily, weekly, biweekly, every 3 weeks, every 4 weeks, monthly, or annually.

26. The method of claim 1, wherein the composition is administered after onset of balance dysfuntion in the patient.


SEQUENCE LISTING (41 sequences)

SEQ ID NO: 1 - DNA, 7483 bp, Homo sapiens
  source 1..7483: mol_type = genomic DNA
agtgctggctggacagctgctctgggcaggagagagagggagagacaagagacacacacagagagacggcgaggaagggaaagacccagagggacgcctagaacgagacttggagccagacagaggaagaggggacgtgtgtttgcagactggctgggcccgtgacccagcttcctgagtcctccgtgcaggtggcagctgtaccaggctggcaggtcactgagagtgggcagctgggccccagaactgtgcctggcccagtgggcagcaggagctcctgacttgggaccatggtgattcttcagcagggggaccatgtgtggatggacctgagattggggcaggagttcgacgtgcccatcggggcggtggtgaagctctgcgactctgggcaggtccaggtggtggatgatgaagacaatgaacactggatctctccgcagaacgcaacgcacatcaagcctatgcaccccacgtcggtccacggcgtggaggacatgatccgcctgggggacctcaacgaggcgggcatcttgcgcaacctgcttatccgctaccgggaccacctcatctacacgtatacgggctccatcctggtggctgtgaacccctaccagctgctctccatctactcgccagagcacatccgccagtataccaacaagaagattggggagatgcccccccacatctttgccattgctgacaactgctacttcaacatgaaacgcaacagccgagaccagtgctgcatcatcagtggggaatctggggccgggaagacggagagcacaaagctgatcctgcagttcctggcagccatcagtgggcagcactcgtggattgagcagcaggtcttggaggccacccccattctggaagcatttgggaatgccaagaccatccgcaatgacaactcaagccgtttcggaaagtacatcgacatccacttcaacaagcggggcgccatcgagggcgcgaagattgagcagtacctgctggaaaagtcacgtgtctgtcgccaggccctggatgaaaggaactaccacgtgttctactgcatgctggagggtatgagtgaggatcagaagaagaagctgggcttgggccaggcctctgactacaactacttggccatgggtaactgcataacctgtgagggccgggtggacagccaggagtacgccaacatccgctccgccatgaaggtgctcatgttcactgacaccgagaactgggagatctcgaagctcctggctgccatcctgcacctgggcaacctgcagtatgaggcacgcacatttgaaaacctggatgcctgtgaggttctcttctccccatcgctggccacagctgcatccctgcttgaggtgaaccccccagacctgatgagctgcctgactagccgcaccctcatcacccgcggggagacggtgtccaccccactgagcagggaacaggcactggacgtgcgcgacgccttcgtaaaggggatctacgggcggctgttcgtgtggattgtggacaagatcaacgcagcaatttacaagcctccctcccaggatgtgaagaactctcgcaggtccatcggcctcctggacatctttgggtttgagaactttgctgtgaacagctttgagcagctctgcatcaacttcgccaatgagcacctgcagcagttctttgtgcggcacgtgttcaagctggagcaggaggaatatgacctggagagcattgactggctgcacatcgagttcactgacaaccaggatgccctggacatgattgccaacaagcccatgaacatcatctccctcatcgatgaggagagcaagttccccaagggcacagacaccaccatgttacacaagctgaactcccagcacaagctcaacgccaactacatcccccccaagaacaaccatgagacccagtttggcatcaaccattttgcaggcatcgtctactatgagacccaaggcttcctggagaagaaccgagacaccctgcatggggacattatccagctggtccactcctccaggaacaagttcatcaagcagatcttccaggccgatgtcgccatgggcgccgagaccaggaagcgctcgcccacacttagcagccagttcaagcggtcactggagctgctgatgcgcacgctgggtgcctgccagcccttctttgtgcgatgcatcaagcccaatgagttcaagaagcccatgctgttcgaccggcacctgtgcgtgcgccagctgcggtactcaggaatgatggagaccatccgaatccgccgagctggctaccccatccgctacagcttcgtagagtttgtggagcggtaccgtgtgctgctgccaggtgtgaagccggcctacaagcagggcgacctccgcgggacttgccagcgcatggctgaggctgtgctgggcacccacgatgactggcagataggcaaaaccaagatctttctgaaggaccaccatgacatgctgctggaagtggagcgggacaaagccatcaccgacagagtcatcctccttcagaaagtcatccggggattcaaagacaggtctaactttctgaagctgaagaacgctgccacactgatccagaggcactggcggggtcacaactgtaggaagaactacgggctgatgcgtctgggcttcctgcggctgcaggccctgcaccgctcccggaagctgcaccagcagtaccgcctggcccgccagcgcatcatccagttccaggcccgctgccgcgcctatctggtgcgcaaggccttccgccaccgcctctgggctgtgctcaccgtgcaggcctatgcccggggcatgatcgcccgcaggctgcaccaacgcctcagggctgagtatctgtggcgcctcgaggctgagaaaatgcggctggcggaggaagagaagcttcggaaggagatgagcgccaagaaggccaaggaggaggccgagcgcaagcatcaggagcgcctggcccagctggctcgtgaggacgctgagcgggagctgaaggagaaggaggccgctcggcggaagaaggagctcctggagcagatggaaagggcccgccatgagcctgtcaatcactcagacatggtggacaagatgtttggcttcctggggacttcaggtggcctgccaggccaggagggccaggcacctagtggctttgaggacctggagcgagggcggagggagatggtggaggaggacctggatgcagccctgcccctgcctgacgaggatgaggaggacctctctgagtataaatttgccaagttcgcggccacctacttccaggggacaaccacgcactcctacacccggcggccactcaaacagccactgctctaccatgacgacgagggtgaccagctggcagccctggcggtctggatcaccatcctccgcttcatgggggacctccctgagcccaagtaccacacagccatgagtgatggcagtgagaagatccctgtgatgaccaagatttatgagaccctgggcaagaagacgtacaagagggagctgcaggccctgcagggcgagggcgaggcccagctccccgagggccagaagaagagcagtgtgaggcacaagctggtgcatttgactctgaaaaagaagtccaagctcacagaggaggtgaccaagaggctgcatgacggggagtccacagtgcagggcaacagcatgctggaggaccggcccacctccaacctggagaagctgcacttcatcatcggcaatggcatcctgcggccagcactccgggacgagatctactgccagatcagcaagcagctgacccacaacccctccaagagcagctatgcccggggctggattctcgtgtctctctgcgtgggctgtttcgccccctccgagaagtttgtcaagtacctgcggaacttcatccacgggggcccgcccggctacgccccgtactgtgaggagcgcctgagaaggacctttgtcaatgggacacggacacagccgcccagctggctggagctgcaggccaccaagtccaagaagccaatcatgttgcccgtgacattcatggatgggaccaccaagaccctgctgacggactcggcaaccacggccaaggagctctgcaacgcgctggccgacaagatctctctcaaggaccggttcgggttctccctctacattgccctgtttgacaaggtgtcctccctgggcagcggcagtgaccacgtcatggacgccatctcccagtgcgagcagtacgccaaggagcagggcgcccaggagcgcaacgccccctggaggctcttcttccgcaaagaggtcttcacgccctggcacagcccctccgaggacaacgtggccaccaacctcatctaccagcaggtggtgcgaggagtcaagtttggggagtacaggtgtgagaaggaggacgacctggctgagctggcctcccagcagtactttgtagactatggctctgagatgatcctggagcgcctcctgaacctcgtgcccacctacatccccgaccgcgagatcacgcccctgaagacgctggagaagtgggcccagctggccatcgccgcccacaagaaggggatttatgcccagaggagaactgatgcccagaaggtcaaagaggatgtggtcagttatgcccgcttcaagtggcccttgctcttctccaggttttatgaagcctacaaattctcaggccccagtctccccaagaacgacgtcatcgtggccgtcaactggacgggtgtgtactttgtggatgagcaggagcaggtacttctggagctgtccttcccagagatcatggccgtgtccagcagcagggagtgccgtgtctggctctcactgggctgctctgatcttggctgtgctgcgcctcactcaggctgggcaggactgaccccggcggggccctgttctccgtgttggtcctgcaggggagcgaaaacgacggcccccagcttcacgctggccaccatcaagggggacgaatacaccttcacctccagcaatgctgaggacattcgtgacctggtggtcaccttcctagaggggctccggaagagatctaagtatgttgtggccctgcaggataaccccaaccccgcaggcgaggagtcaggcttcctcagctttgccaagggagacctcatcatcctggaccatgacacgggcgagcaggtcatgaactcgggctgggccaacggcatcaatgagaggaccaagcagcgtggggacttccccaccgacagtgtgtacgtcatgcccactgtcaccatgccaccgcgggagattgtggccctggtcaccatgactcccgatcagaggcaggacgttgtccggctcttgcagctgcgaacggcggagcccgaggtgcgtgccaagccctacacgctggaggagttttcctatgactacttcaggcccccacccaagcacacgctgagccgtgtcatggtgtccaaggcccgaggcaaggaccggctgtggagccacacgcgggaaccgctcaagcaggcgctgctcaagaagctcctgggcagtgaggagctctcgcaggaggcctgcctggccttcattgctgtgctcaagtacatgggcgactacccgtccaagaggacacgctccgtcaacgagctcaccgaccagatctttgagggtcccctgaaagccgagcccctgaaggacgaggcatatgtgcagatcctgaagcagctgaccgacaaccacatcaggtacagcgaggagcggggttgggagctgctctggctgtgcacgggccttttcccacccagcaacatcctcctgccccacgtgcagcgcttcctgcagtcccgaaagcactgcccactcgccatcgactgcctgcaacggctccagaaagccctgagaaacgggtcccggaagtaccctccgcacctggtggaggtggaggccatccagcacaagaccacccagattttccacaaagtctacttccctgatgacactgacgaggccttcgaagtggagtccagcaccaaggccaaggacttctgccagaacatcgccaccaggctgctcctcaagtcctcagagggattcagcctctttgtcaaaattgcagacaaggtcctcagcgttcctgagaatgacttcttctttgactttgttcgacacttgacagactggataaagaaagctcggcccatcaaggacggaattgtgccctcactcacctaccaggtgttcttcatgaagaagctgtggaccaccacggtgccagggaaggatcccatggccgattccatcttccactattaccaggagttgcccaagtatctccgaggctaccacaagtgcacgcgggaggaggtgctgcagctgggggcgctgatctacagggtcaagttcgaggaggacaagtcctacttccccagcatccccaagctgctgcgggagctggtgccccaggaccttatccggcaggtctcacctgatgactggaagcggtccatcgtcgcctacttcaacaagcacgcagggaagtccaaggaggaggccaagctggccttcctgaagctcatcttcaagtggcccacctttggctcagccttcttcgaggtgaagcaaactacggagccaaacttccctgagatcctcctaattgccatcaacaagtatggggtcagcctcatcgatcccaaaacgaaggatatcctcaccactcatcccttcaccaagatctccaactggagcagcggcaacacctacttccacatcaccattgggaacttggtgcgcgggagcaaactgctctgcgagacgtcactgggctacaagatggatgacctcctgacttcctacattagccagatgctcacagccatgagcaaacagcggggctccaggagcggcaagtgaacagtcacggggaggtgctggttccatgcctgctctcgaggcagcagtgggttcaggcccatcagctacccctgcagctggggaagacttatgccatcccggcagcgaggctgggctggccagccaccactgactataccaactgggcctctgatgttcttccagtgaggcatctctctgggatgcagaacttccctccatccacccctctggcacctgggttggtctaatcctagtttgctgtggccttcccggttgtgagagcctgtgatccttagatgtgtctcctgtttcagaccagccccaccatgcaacttcctttgactttctgtgtaccactgggatagaggaatcaagaggacaatctagctctccatactttgaacaaccaaatgtgcattgaatactctgaaaccgaagggactggatctgcaggtgggatgagggagacagaccacttttctatattgcagtgtgaatgctgggcccctgctcaagtctaccctgatcacctcagggcataaagcatgtttcattctctggcc

SEQ ID NO: 2 - DNA, 401 bp, synthetic construct
  source 1..401: mol_type = other DNA
agctagctgcagtaacgccattttgcaaggcatggaaaaataccaaaccaagaatagagaagttcagatcaagggcgggtacatgaaaatagctaacgttgggccaaacaggatatctgcggtgagcagtttcggccccggcccggggccaagaacagatggtcaccgcagtttcggccccggcccgaggccaagaacagatggtccccagatatggcccaaccctcagcagtttcttaagacccatcagatgtttccaggctcccccaaggacctgaaatgaccctgcgccttatttgaattaaccaatcagcctgcttctcgcttctgttcgcgcgcttctgcttcccgagctctataaaagagctcacaacccctcactcggcgcgccagtcctccga

SEQ ID NO: 3 - DNA, 232 bp, synthetic construct
  source 1..232: mol_type = other DNA
ttaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacaataaac

SEQ ID NO: 4 - DNA, 181 bp, synthetic construct
  source 1..181: mol_type = other DNA
ggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcag

SEQ ID NO: 5 - skipped

SEQ ID NO: 6 - DNA, 45 bp, synthetic construct
  source 1..45: mol_type = other DNA
tgagtacgccaaaaattttgactagcggaggctagaaggagagag

SEQ ID NO: 7 - DNA, 242 bp, synthetic construct
  source 1..242: mol_type = other DNA
ggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttg

SEQ ID NO: 8 - DNA, 124 bp, synthetic construct
  source 1..124: mol_type = other DNA
aacttttaaaagaaaaggggggattggggggtacagtgcaggggaaagaatagtagacataatagcaacagacatacaaactaaagaattacaaaaacaaattacaaaaattcaaaattttatc

SEQ ID NO: 9 - DNA, 23 bp, synthetic construct
  source 1..23: mol_type = other DNA
tagtgaatagagttaggcaggga

SEQ ID NO: 10 - DNA, 599 bp, synthetic construct
  source 1..599: mol_type = other DNA
tcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgga

SEQ ID NO: 11 - DNA, 150 bp, synthetic construct
  source 1..150: mol_type = other DNA
tggaagggctaattcactcccaacgaagacaagatctgctttttgcttgtactgggtctctctggttagaccagatctgagcctgggagctctctggctaactagggaacccactgcttaagcctcaataaagcttgccttgagtgcttc

SEQ ID NO: 12 - DNA, 96 bp, synthetic construct
  source 1..96: mol_type = other DNA
ggtctctctggttagaccagatctgagcctgggagctctctggctaactagggaacccactgcttaagcctcaataaagcttgccttgagtgcttc

SEQ ID NO: 13 - DNA, 18 bp, synthetic construct
  source 1..18: mol_type = other DNA
tggcgcccgaacagggac

SEQ ID NO: 14 - DNA, 366 bp, synthetic construct
  source 1..366: mol_type = other DNA
gatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgat

SEQ ID NO: 15 - DNA, 588 bp, synthetic construct
  source 1..588: mol_type = other DNA
gcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaacc

SEQ ID NO: 16 - DNA, 20 bp, synthetic construct
  source 1..20: mol_type = other DNA
catgtccaaggtcctcttcc

SEQ ID NO: 17 - DNA, 20 bp, synthetic construct
  source 1..20: mol_type = other DNA
cctagaatcagtgcagagca

SEQ ID NO: 18 - DNA, 288 bp, synthetic construct
  source 1..288: mol_type = other DNA
acataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatc

SEQ ID NO: 19 - DNA, 1536 bp, Vesiculovirus indiana
  source 1..1536: mol_type = genomic DNA
atgaagtgccttttgtacttagcctttttattcattggggtgaattgcaagttcaccatagtttttccacacaaccaaaaaggaaactggaaaaatgttccttctaattaccattattgcccgtcaagctcagatttaaattggcataatgacttaataggcacagccttacaagtcaaaatgcccaagagtcacaaggctattcaagcagacggttggatgtgtcatgcttccaaatgggtcactacttgtgatttccgctggtatggaccgaagtatataacacattccatccgatccttcactccatctgtagaacaatgcaaggaaagcattgaacaaacgaaacaaggaacttggctgaatccaggcttccctcctcaaagttgtggatatgcaactgtgacggatgccgaagcagtgattgtccaggtgactcctcaccatgtgctggttgatgaatacacaggagaatgggttgattcacagttcatcaacggaaaatgcagcaattacatatgccccactgtccataactctacaacctggcattctgactataaggtcaaagggctatgtgattctaacctcatttccatggacatcaccttcttctcagaggacggagagctatcatccctgggaaaggagggcacagggttcagaagtaactactttgcttatgaaactggaggcaaggcctgcaaaatgcaatactgcaagcattggggagtcagactcccatcaggtgtctggttcgagatggctgataaggatctctttgctgcagccagattccctgaatgcccagaagggtcaagtatctctgctccatctcagacctcagtggatgtaagtctaattcaggacgttgagaggatcttggattattccctctgccaagaaacctggagcaaaatcagagcgggtcttccaatctctccagtggatctcagctatcttgctcctaaaaacccaggaaccggtcctgctttcaccataatcaatggtaccctaaaatactttgagaccagatacatcagagtcgatattgctgctccaatcctctcaagaatggtcggaatgatcagtggaactaccacagaaagggaactgtgggatgactgggcaccatatgaagacgtggaaattggacccaatggagttctgaggaccagttcaggatataagtttcctttatacatgattggacatggtatgttggactccgatcttcatcttagctcaaaggctcaggtgttcgaacatcctcacattcaagacgctgcttcgcaacttcctgatgatgagagtttattttttggtgatactgggctatccaaaaatccaatcgagcttgtagaaggttggttcagtagttggaaaagctctattgcctcttttttctttatcatagggttaatcattggactattcttggttctccgagttggtatccatctttgcattaaattaaagcacaccaagaaaagacagatttatacagacatagagatgaaccgacttggaaagtaa

SEQ ID NO: 20 - protein, 511 aa, Vesiculovirus indiana
MKCLLYLAFLFIGVNCKFTIVFPHNQKGNWKNVPSNYHYCPSSSDLNWHNDLIGTALQVKMPKSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 21 - DNA, 1554 bp, Vesiculovirus newjersey
  source 1..1554: mol_type = genomic DNA
atgttgtcttatctaatctttgcacttgccgtttcgcccattttgggcaaaattgaaattgtgtttcctcaacataccactggggattggaagagagttccccatgaatataattattgccctaccagcgcagacaagaactcacatgggactcaaacaggaatccctgttgagttaacaatgccaaaaggactaacaacccatcaagttgaaggatttatgtgtcactcagccttgtggatgaccacttgtgacttcagatggtatgggccaaaatacataacccattccatacataatgaagagcctacagattatcaatgtttggaggccattaagtcatacaaagatggagtcagtttcaatccagggtttcctcctcagagctgcgggtatggcacagttaccgatgccgaagcccatattgtgacagttactccccactctgtcaaagtggacgagtacacgggggaatggatcgatccacatttcatcggaggaaggtgcaaaggacaaatttgtgaaacagtccataattccacaaaatggtttacgtcctctgatggagaaagtgtctgcagtcaattgtttactttggttggaggaatttttttctctgattcagaagagattacctccatggggttaccagaaacaggaatcagaagtaattacttcccctacatatctacagagggaatttgcaaaatgccgttttgcagaaaacaggggtacaagcttaaaaatgacctctggttccagatcatggacccagacctggataaaacggttagagatctccctcatattaaggactgtgacctctcctcgtccataatcacaccaggagaacatgctacagacatctcactgatatcagatgttgaaaggatcctggactatgctctttgtcagaatacatggagtaaaattgaatcgggagaaccaattactccggtagatctcagctatcttgggccaaaaaacccaggggttgggccggtcttcaccatcattaacggttccctgcattattttacatcgaagtatctgcgagtcgaattagaaagtcctgtcatacccagaatggaaggaaaagttgcaggaactaggattgtacggcaattgtgggatcagtggtttcctttcggagaagttgagattggacccaatggtgtgttgaaaacgaagcaagggtataaattcccactacacatcattggaactggagaagtagacagtgacatcaaaatggaaagggttgtcaagcactgggaacacccccatattgaggccgctcagacatttttaaaaaaagatgacacaggagaagtcctttattatggcgacaccggagtgtcgaaaaatccagttgaattagtcgagggatggtttagtggatggaggagctccctcatgggagtgctggctgtgattataggatttgtgattttaatgtttttaattaaattgattggagtcttatctagccttttcagacctaaacgcaggccaatctacaaatcagacgtggaaatggctcatttccgttaa

SEQ ID NO: 22 - protein, 517 aa, Vesiculovirus newjersey
MLSYLIFALAVSPILGKIEIVFPQHTTGDWKRVPHEYNYCPTSADKNSHGTQTGIPVELTMPKGLTTHQVEGFMCHSALWMTTCDFRWYGPKYITHSIHNEEPTDYQCLEAIKSYKDGVSFNPGFPPQSCGYGTVTDAEAHIVTVTPHSVKVDEYTGEWIDPHFIGGRCKGQICETVHNSTKWFTSSDGESVCSQLFTLVGGIFFSDSEEITSMGLPETGIRSNYFPYISTEGICKMPFCRKQGYKLKNDLWFQIMDPDLDKTVRDLPHIKDCDLSSSIITPGEHATDISLISDVERILDYALCQNTWSKIESGEPITPVDLSYLGPKNPGVGPVFTIINGSLHYFTSKYLRVELESPVIPRMEGKVAGTRIVRQLWDQWFPFGEVEIGPNGVLKTKQGYKFPLHIIGTGEVDSDIKMERVVKHWEHPHIEAAQTFLKKDDTGEVLYYGDTGVSKNPVELVEGWFSGWRSSLMGVLAVIIGFVILMFLIKLIGVLSSLFRPKRRPIYKSDVEMAHFR

SEQ ID NO: 23 - DNA, 1536 bp, unidentified
  source 1..1536: mol_type = unassigned DNA
atgaagtgccttttgtacttagcctttttattcattggggtgaattgcaagttcaccatagtttttccacacaaccaaaaaggaaactggaaaaatgttccttctaattaccattattgcccgtcaagctcagatttaaattggcataatgacttaataggcacagccttacaagtcaaaatgcccaagagtcacaaggctattcaagcagacggttggatgtgtcatgcttccaaatgggtcactacttgtgatttccgctggtatggaccgaagtatataacacattccatccgatccttcactccatctgtagaacaatgcaaggaaagcattgaacaaacgaaacaaggaacttggctgaatccaggcttccctcctcaaagttgtggatatgcaactgtgacggatgccgaagcagtgattgtccaggtgactcctcaccatgtgctggttgatgaatacacaggagaatgggttgattcacagttcatcaacggaaaatgcagcaattacatatgccccactgtccataactctacaacctggcattctgactataaggtcaaagggctatgtgattctaacctcatttccatggacatcaccttcttctcagaggacggagagctatcatccctgggaaaggagggcacagggttcagaagtaactactttgcttatgaaaatggaggcaaggcctgcaaaatgcaatactgcaagcattggggagtcagactcccatcaggtgtctggttcgagatggctgataaggatctctttgctgcagccagattccctgaatgcccagaagggtcaagtatctctgctccatctcagacctcagtggatgtaagtctaattcaggacgttgagaggatcttggattattccctctgccaagaaacctggagcaaaatcagagcgggtcttccaatctctccagtggatctcagctatcttgctcctaaaaacccaggaaccggtcctgctttcaccataatcaatggtaccctaaaatactttgagaccagatacatcagagtcgatattgctgctccaatcctctcaagaatggtcggaatgatcagtggaactaccgcagaaagggaactgtgggatgactgggcaccatatgaagacgtggaaattggacccaatggagttctgaggaccagttcaggatataagtttcctttatacatgattggacatggtatgttggactccgatcttcatcttagctcaaaggctcaggtgttcgaacatcctcacattcaagacgctgcttcgcaacttcctgatgatgagagtttattttttggtgatactgggctatccaaaaatccaatcgagcttgtagaaggttggttcagtagttggaaaagctctattgcctcttttttctttatcatagggttaatcattggactattcttggttctccgagttggtatccatctttgcattaaattaaagcacaccaagaaaagacagatttatacagacatagagatgaaccgacttggaaagtaa

SEQ ID NO: 24 - protein, 511 aa, unidentified
MKCLLYLAFLFIGVNCKFTIVFPHNQKGNWKNVPSNYHYCPSSSDLNWHNDLIGTALQVKMPKSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYENGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTAERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 25 - DNA, 1539 bp, unidentified
  source 1..1539: mol_type = unassigned DNA
atgaatttcctactcttgacatttattgtgttgccgttgtgcagccacgccaagttctccattgtattccctcaaagccaaaaaggcaattggaagaatgtaccatcatcttaccattactgcccttcaagttcggatcaaaactggcacaatgatttgcttggaatcacaatgaaagtcaaaatgcccaaaacacacaaagctattcaagcagacgggtggatgtgtcatgctgccaaatggatcactacctgtgactttcgctggtacggacccaaatacatcactcactccattcattccatccagcctacttcagagcagtgtaaagaaagcatcaagcaaacaaaacaaggtacttggatgagtcctggcttccctccacagaactgcgggtatgcaacagtaacagactctgtcgctgttgtcgtccaagccactcctcatcatgtcttggttgatgaatatactggagaatggatcgactctcaattccccaacgggaaatgtgaaaccgaagagtgcgagaccgtccacaactctaccgtatggtactctgactacaaagtaactggattatgtgacgcaactctggtagacacagagatcaccttcttctctgaagatggcaaaaaagaatctatcgggaagcccaacacaggctataggagcaactacttcgcttatgagaaaggggacaaagtatgtaaaatgaactactgcaagcatgcgggtgtgaggttgccttccggggtttggtttgagtttgtggatcaggatgtctacgccgccgccaaacttccagaatgccccgttggtgccactatctccgctccgacacagacctctgttgacgtaagtctcattctagatgtagagagaattttagattactctctgtgtcaagagacatggagcaagatccggtccaaacagccagtatcccctgttgaccttagttacttggcccccaagaatcctgggaccggaccggcattcacaatcatcaatggcactctgaagtactttgagaccagatacattcggattgatatagacaatccaatcatctccaagatggtggggaaaataagtggcagtcaaacagaacgagaattgtggacagagtggttcccctacgagggtgtcgagatagggccaaatgggattctcaaaacccctacaggatacaaattcccactcttcatgataggacacgggatgctagattccgacttgcacaagacgtcccaagcagaggtctttgaacatcctcaccttgcagaagcaccaaagcagttgccggaggaggagactttattttttggtgacacaggaatctccaaaaatccggtcgaactgattgaagggtggtttagtagttggaagagcactgtagtcacctttttctttgccataggagtatttatactactgtatgtagtggccagaattgtgatcgcagtgagatacagatatcaaggctcaaataacaaaagaatttacaatgatattgagatgagcagatttagaaaatga

SEQ ID NO: 26 - protein, 512 aa, unidentified
MNFLLLTFIVLPLCSHAKFSIVFPQSQKGNWKNVPSSYHYCPSSSDQNWHNDLLGITMKVKMPKTHKAIQADGWMCHAAKWITTCDFRWYGPKYITHSIHSIQPTSEQCKESIKQTKQGTWMSPGFPPQNCGYATVTDSVAVVVQATPHHVLVDEYTGEWIDSQFPNGKCETEECETVHNSTVWYSDYKVTGLCDATLVDTEITFFSEDGKKESIGKPNTGYRSNYFAYEKGDKVCKMNYCKHAGVRLPSGVWFEFVDQDVYAAAKLPECPVGATISAPTQTSVDVSLILDVERILDYSLCQETWSKIRSKQPVSPVDLSYLAPKNPGTGPAFTIINGTLKYFETRYIRIDIDNPIISKMVGKISGSQTERELWTEWFPYEGVEIGPNGILKTPTGYKFPLFMIGHGMLDSDLHKTSQAEVFEHPHLAEAPKQLPEEETLFFGDTGISKNPVELIEGWFSSWKSTVVTFFFAIGVFILLYVVARIVIAVRYRYQGSNNKRIYNDIEMSRFRK

SEQ ID NO: 27 - DNA, 1539 bp, unidentified
  source 1..1539: mol_type = unassigned DNA
atgttgagactttttctcttttgtttcttggccttaggagcccactccaaatttactatagtattccctcatcatcaaaaagggaattggaagaatgtgccttccacatatcattattgcccttctagttctgaccagaattggcataatgatttgactggagttagtcttcatgtgaaaattcccaaaagtcacaaagctatacaagcagatggctggatgtgccacgctgctaaatgggtgactacttgtgacttcagatggtacggacccaaatacatcacgcattccatacactctatgtcacccaccctagaacagtgcaagaccagtattgagcagacaaagcaaggagtttggattaatccaggctttccccctcaaagctgcggatatgctacagtgacggatgcagaggtggttgttgtacaagcaacacctcatcatgtgttggttgatgagtacacaggagaatggattgactcacaattggtggggggcaaatgttccaaggaggtttgtcaaacggttcacaactcgaccgtgtggcatgctgattacaagattacagggctgtgcgagtcaaatctggcatcagtggatatcaccttcttctctgaggatggtcaaaagacgtctttgggaaaaccgaacactggattcaggagtaatcactttgcttacgaaagtggagagaaggcatgccgtatgcagtactgcacacaatgggggatccgactaccttctggagtatggtttgaattagtggacaaagatctcttccaggcggcaaaattgcctgaatgtcctagaggatccagtatctcagctccttctcagacttctgtggatgttagtttgatacaagacgtagagaggatcttagattactctctatgccaggagacgtggagtaagatacgagccaagcttcctgtatctccagtagatctgagttatctcgccccaaaaaatccagggagcggaccggccttcactatcattaatggcactttgaaatatttcgaaacaagatacatcagagttgacataagtaatcccatcatccctcacatggtgggaacaatgagtggaaccacgactgagcgtgaattgtggaatgattggtatccatatgaagacgtagagattggtccaaatggggtgttgaaaactcccactggtttcaagtttccgctgtacatgattgggcacggaatgttggattccgatctccacaaatcctcccaggctcaagtcttcgaacatccacacgcaaaggacgctgcatcacagcttcctgatgatgagactttattttttggtgacacaggactatcaaaaaacccagtagagttagtagaaggctggttcagtagctggaagagcacattggcatcgttctttctgattataggcttgggggttgcattaatcttcatcattcgaattattgttgcgattcgctataaatacaaggggaggaagacccaaaaaatttacaatgatgtcgagatgagtcgattgggaaataaataa

SEQ ID NO: 28 - protein, 512 aa, unidentified
MLRLFLFCFLALGAHSKFTIVFPHHQKGNWKNVPSTYHYCPSSSDQNWHNDLTGVSLHVKIPKSHKAIQADGWMCHAAKWVTTCDFRWYGPKYITHSIHSMSPTLEQCKTSIEQTKQGVWINPGFPPQSCGYATVTDAEVVVVQATPHHVLVDEYTGEWIDSQLVGGKCSKEVCQTVHNSTVWHADYKITGLCESNLASVDITFFSEDGQKTSLGKPNTGFRSNHFAYESGEKACRMQYCTQWGIRLPSGVWFELVDKDLFQAAKLPECPRGSSISAPSQTSVDVSLIQDVERILDYSLCQETWSKIRAKLPVSPVDLSYLAPKNPGSGPAFTIINGTLKYFETRYIRVDISNPIIPHMVGTMSGTTTERELWNDWYPYEDVEIGPNGVLKTPTGFKFPLYMIGHGMLDSDLHKSSQAQVFEHPHAKDAASQLPDDETLFFGDTGLSKNPVELVEGWFSSWKSTLASFFLIIGLGVALIFIIRIIVAIRYKYKGRKTQKIYNDVEMSRLGNK

SEQ ID NO: 29 - DNA, 1698 bp, unidentified
  source 1..1698: mol_type = unassigned DNA
atgaaactcccaacaggaatggtcattttatgtagcctaataatagttcgggcagggtttgacgacccccgcaaggctatcgcattagtacaaaaacaacatggtaaaccatgcgaatgcagcggagggcaggtatccgaggccccaccgaactccatccaacaggtaacttgcccaggcaagacggcctacttaatgaccaaccaaaaatggaaatgcagagtcactccaaaaaatctcacccctagcgggggagaactccagaactgcccctgtaacactttccaggactcgatgcacagttcttgttatactgaataccggcaatgcagggcgaataataagacatactacacggccaccttgcttaaaatacggtctgggagcctcaacgaggtacagatattacaaaaccccaatcagctcctacagtccccttgtaggggctctataaatcagcccgtttgctggagtgccacagcccccatccatatctccgatggtggaggacccctcgatactaagagagtgtggacagtccaaaaaaggctagaacaaattcataaggctatgcatcctgaacttcaataccaccccttagccctgcccaaagtcagagatgaccttagccttgatgcacggacttttgatatcctgaataccacttttaggttactccagatgtccaattttagccttgcccaagattgttggctctgtttaaaactaggtacccctacccctcttgcgatacccactccctctttaacctactccctagcagactccctagcgaatgcctcctgtcagattatacctcccctcttggttcaaccgatgcagttctccaactcgtcctgtttatcttcccctttcattaacgatacggaacaaatagacttaggtgcagtcacctttactaactgcacctctgtagccaatgtcagtagtcctttatgtgccctaaacgggtcagtcttcctctgtggaaataacatggcatacacctatttaccccaaaactggacaggactttgcgtccaagcctccctcctccccgacattgacatcatcccgggggatgagccagtccccattcctgccattgatcattatatacatagacctaaacgagctgtacagttcatccctttactagctggactgggaatcaccgcagcattcaccaccggagctacaggcctaggtgtctccgtcacccagtatacaaaattatcccatcagttaatatctgatgtccaagtcttatccggtaccatacaagatttacaagaccaggtagactcgttagctgaagtagttctccaaaataggaggggactggacctactaacggcagaacaaggaggaatttgtttagccttacaagaaaaatgctgtttttatgctaacaagtcaggaattgtgagaaacaaaataagaaccctacaagaagaattacaaaaacgcagggaaagcctggcatccaaccctctctggaccgggctgcagggctttcttccgtacctcctacctctcctgggacccctactcaccctcctactcatactaaccattgggccatgcgttttcagtcgcctcatggccttcattaatgatagacttaatgttgtacatgccatggtgctggcccagcaataccaagcactcaaagctgaggaagaagctcaggattga

SEQ ID NO: 30 - protein, 565 aa, unidentified
MKLPTGMVILCSLIIVRAGFDDPRKAIALVQKQHGKPCECSGGQVSEAPPNSIQQVTCPGKTAYLMTNQKWKCRVTPKNLTPSGGELQNCPCNTFQDSMHSSCYTEYRQCRANNKTYYTATLLKIRSGSLNEVQILQNPNQLLQSPCRGSINQPVCWSATAPIHISDGGGPLDTKRVWTVQKRLEQIHKAMHPELQYHPLALPKVRDDLSLDARTFDILNTTFRLLQMSNFSLAQDCWLCLKLGTPTPLAIPTPSLTYSLADSLANASCQIIPPLLVQPMQFSNSSCLSSPFINDTEQIDLGAVTFTNCTSVANVSSPLCALNGSVFLCGNNMAYTYLPQNWTGLCVQASLLPDIDIIPGDEPVPIPAIDHYIHRPKRAVQFIPLLAGLGITAAFTTGATGLGVSVTQYTKLSHQLISDVQVLSGTIQDLQDQVDSLAEVVLQNRRGLDLLTAEQGGICLALQEKCCFYANKSGIVRNKIRTLQEELQKRRESLASNPLWTGLQGFLPYLLPLLGPLLTLLLILTIGPCVFSRLMAFINDRLNVVHAMVLAQQYQALKAEEEAQD

SEQ ID NO: 31 - DNA, 1692 bp, unidentified
  source 1..1692: mol_type = unassigned DNA
atgggattcacaacaaagataatcttcttatacaacctagtactggtctacgcggggtttgacgaccctcgcaaagccatagaactagtacaaaagcgatatggccgaccatgcgattgcagcggaggacaagtgtccgagcccccgtcagacagggtcagtcaagtgacttgctcaggcaagacagcttacttaatgcccgaccaaagatggaaatgtaagtcaattccaaaagacacctccccaagcgggccactccaagagtgcccctgtaattcttaccagtcctcagtacacagttcttgttatacctcataccaacaatgcagatcaggcaataagacatattatacggctactctgctaaaaacacaaactgggggcaccagtgatgtacaagtattaggatccaccaacaaacttatacaatctccctgtaatggcataaaagggcagtctatttgctggagcactacagctcctatccacgtctctgatggaggaggtccattagacaccacaagaattaaaagtgttcagagaaaactggaagaaattcataaagccctatatcctgaacttcagtatcaccctttggccatacctaaggttagagataacctcatggtcgatgcccagactttaaacattctcaatgccacttacaacttactcctaatgtccaacacgagcctagtggacgactgttggctttgtttaaaattaggtccccctactcccctcgcaatacctaacttcctattatcctacgtgactcgctcctcggataatatctcttgtttaataattcccccccttctagttcaaccgatgcagttttccaattcatcttgcctcttttccccctcctacaacagtacagaagaaatagatctaggccatgttgccttcagcaactgtacctccataaccaatgtcaccggtcccatatgcgctgtaaatggttcggtctttctctgtggcaataacatggcatacacttatctacccacgaactggacggggctttgcgtcctagcaactctcctccccgacattgacatcattcccggagatgaaccggtccccatccctgctattgatcattttatatatagacctaaacgggccatacagtttattcctttactagcagggctagggatcaccgcagccttcacaacaggagctacaggcctaggtgtctctgtgacccaatatacaaaattatctaatcagctaatttctgatgtacaaatcttatctagcaccatacaagatctgcaagatcaagtagactcattagccgaagtggttctccagaacagaagggggctagatctacttacagcagaacaaggaggaatctgtttagccctgcaagaaaaatgctgcttttatgttaacaagtcagggattgtgagagacaaaataaaaaccttacaagaagaactagaaagacgtagaaaagatctagcttccaacccactttggactgggcttcaagggctcctcccttacctcctgccctttcttggccctctacttaccctcctgctcttactcaccattgggccgtgcatttttaaccgtctaaccgcttttattaatgataagttaaacataatacacgctatggtgctaacccaacagtatcaggtgctcagaaccgatgaagaagctcaagattga

SEQ ID NO: 32 - protein, 563 aa, unidentified
MGFTTKIIFLYNLVLVYAGFDDPRKAIELVQKRYGRPCDCSGGQVSEPPSDRVSQVTCSGKTAYLMPDQRWKCKSIPKDTSPSGPLQECPCNSYQSSVHSSCYTSYQQCRSGNKTYYTATLLKTQTGGTSDVQVLGSTNKLIQSPCNGIKGQSICWSTTAPIHVSDGGGPLDTTRIKSVQRKLEEIHKALYPELQYHPLAIPKVRDNLMVDAQTLNILNATYNLLLMSNTSLVDDCWLCLKLGPPTPLAIPNFLLSYVTRSSDNISCLIIPPLLVQPMQFSNSSCLFSPSYNSTEEIDLGHVAFSNCTSITNVTGPICAVNGSVFLCGNNMAYTYLPTNWTGLCVLATLLPDIDIIPGDEPVPIPAIDHFIYRPKRAIQFIPLLAGLGITAAFTTGATGLGVSVTQYTKLSNQLISDVQILSSTIQDLQDQVDSLAEVVLQNRRGLDLLTAEQGGICLALQEKCCFYVNKSGIVRDKIKTLQEELERRRKDLASNPLWTGLQGLLPYLLPFLGPLLTLLLLLTIGPCIFNRLTAFINDKLNIIHAMVLTQQYQVLRTDEEAQD

SEQ ID NO: 33 - DNA, 2058 bp, Gibbon ape leukemia virus
  source 1..2058: mol_type = genomic DNA
atggtattgctgcctgggtccatgcttctcacctcaaacctgcaccaccttcggcaccagatgagtcctgggagctggaaaagactgatcatcctcttaagctgcgtattcggcggcggcgggacgagtctgcaaaataagaacccccaccagcccatgaccctcacttggcaggtactgtcccaaactggagacgttgtctgggatacaaaggcagtccagcccccttggacttggtggcccacacttaaacctgatgtatgtgccttggcggctagtcttgagtcctgggatatcccgggaaccgatgtctcgtcctctaaacgagtcagacctccggactcagactatactgccgcttataagcaaatcacctggggagccatagggtgcagctaccctcgggctaggactagaatggcaagctctaccttctacgtatgtccccgggatggccggaccctttcagaagctagaaggtgcggggggctagaatccctatactgtaaagaatgggattgtgagaccacggggaccggttattggctatctaaatcctcaaaagacctcataactgtaaaatgggaccaaaatagcgaatggactcaaaaatttcaacagtgtcaccagaccggctggtgtaacccccttaaaatagatttcacagacaaaggaaaattatccaaggactggataacgggaaaaacctggggattaagattctatgtgtctggacatccaggcgtacagttcaccattcgcttaaaaatcaccaacatgccagctgtggcagtaggtcctgacctcgtccttgtggaacaaggacctcctagaacgtccctcgctctcccacctcctcttcccccaagggaagcgccaccgccatctctccccgactctaactccacagccctggcgactagtgcacaaactcccacggtgagaaaaacaattgttaccctaaacactccgcctcccaccacaggcgacagactttttgatcttgtgcagggggccttcctaaccttaaatgctaccaacccaggggccactgagtcttgctggctttgtttggccatgggccccccttattatgaagcaatagcctcatcaggagaggtcgcctactccaccgaccttgaccggtgccgctgggggacccaaggaaagctcaccctcactgaggtctcaggacacgggttgtgcataggaaaggtgccctttacccatcagcatctctgcaatcagaccctatccatcaattcctccggagaccatcagtatctgctcccctccaaccatagctggtgggcttgcagcactggcctcaccccttgcctctccacctcagtttttaatcagactagagatttctgtatccaggtccagctgattcctcgcatctattactatcctgaagaagttttgttacaggcctatgacaattctcaccccaggactaaaagagaggctgtctcacttaccctagctgttttactggggttgggaatcacggcgggaataggtactggttcaactgccttaattaaaggacctatagacctccagcaaggcctgacaagcctccagatcgccatagatgctgacctccgggccctccaagactcagtcagcaagttagaggactcactgacttccctgtccgaggtagtgctccaaaataggagaggccttgacttgctgtttctaaaagaaggtggcctctgtgcggccctaaaggaagagtgctgtttttacatagaccactcaggtgcagtacgggactccatgaaaaaactcaaagaaaaactggataaaagacagttagagcgccagaaaagccaaaactggtatgaaggatggttcaataactccccttggttcactaccctgctatcaaccatcgctgggcccctattactcctccttctgttgctcatcctcgggccatgcatcatcaataagttagttcaattcatcaatgataggataagtgcagttaaaattctggtccttagacaaaaatatcaggccctagagaacgaaggtaacctttaa

SEQ ID NO: 34 - protein, 685 aa, Gibbon ape leukemia virus
MVLLPGSMLLTSNLHHLRHQMSPGSWKRLIILLSCVFGGGGTSLQNKNPHQPMTLTWQVLSQTGDVVWDTKAVQPPWTWWPTLKPDVCALAASLESWDIPGTDVSSSKRVRPPDSDYTAAYKQITWGAIGCSYPRARTRMASSTFYVCPRDGRTLSEARRCGGLESLYCKEWDCETTGTGYWLSKSSKDLITVKWDQNSEWTQKFQQCHQTGWCNPLKIDFTDKGKLSKDWITGKTWGLRFYVSGHPGVQFTIRLKITNMPAVAVGPDLVLVEQGPPRTSLALPPPLPPREAPPPSLPDSNSTALATSAQTPTVRKTIVTLNTPPPTTGDRLFDLVQGAFLTLNATNPGATESCWLCLAMGPPYYEAIASSGEVAYSTDLDRCRWGTQGKLTLTEVSGHGLCIGKVPFTHQHLCNQTLSINSSGDHQYLLPSNHSWWACSTGLTPCLSTSVFNQTRDFCIQVQLIPRIYYYPEEVLLQAYDNSHPRTKREAVSLTLAVLLGLGITAGIGTGSTALIKGPIDLQQGLTSLQIAIDADLRALQDSVSKLEDSLTSLSEVVLQNRRGLDLLFLKEGGLCAALKEECCFYIDHSGAVRDSMKKLKEKLDKRQLERQKSQNWYEGWFNNSPWFTTLLSTIAGPLLLLLLLLILGPCIINKLVQFINDRISAVKILVLRQKYQALENEGNL

SEQ ID NO: 35 - DNA, 1965 bp, Murine leukemia virus
  source 1..1965: mol_type = genomic DNA
atggcgcgttcaacgctctcaaaaccccctcaagataagattaacccgtggaagcccttaatagtcatgggagtcctgttaggagtagggatggcagagagcccccatcaggtctttaatgtaacctggagagtcaccaacctgatgactgggcgtaccgccaatgccacctccctcctgggaactgtacaagatgccttcccaaaattatattttgatctatgtgatctggtcggagaggagtgggacccttcagaccaggaaccgtatgtcgggtatggctgcaagtaccccgcagggagacagcggacccggacttttgacttttacgtgtgccctgggcataccgtaaagtcggggtgtgggggaccaggagagggctactgtggtaaatgggggtgtgaaaccaccggacaggcttactggaagcccacatcatcgtgggacctaatctcccttaagcgcggtaacaccccctgggacacgggatgctctaaagttgcctgtggcccctgctacgacctctccaaagtatccaattccttccaaggggctactcgagggggcagatgcaaccctctagtcctagaattcactgatgcaggaaaaaaggctaactgggacgggcccaaatcgtggggactgagactgtaccggacaggaacagatcctattaccatgttctccctgacccggcaggtccttaatgtgggaccccgagtccccatagggcccaacccagtattacccgaccaaagactcccttcctcaccaatagagattgtaccggctccacagccacctagccccctcaataccagttaccccccttccactaccagtacaccctcaacctcccctacaagtccaagtgtcccacagccacccccaggaactggagatagactactagctctagtcaaaggagcctatcaggcgcttaacctcaccaatcccgacaagacccaagaatgttggctgtgcttagtgtcgggacctccttattacgaaggagtagcggtcgtgggcacttataccaatcattccaccgctccggccaactgtacggccacttcccaacataagcttaccctatctgaagtgacaggacagggcctatgcatgggggcagtacctaaaactcaccaggccttatgtaacaccacccaaagcgccggctcaggctcctactaccttgcagcacccgccggaacaatgtgggcttgcagcactggattgactccctgcttgtccaccacggtgctcaatctaaccacagattattgtgtattagttgaactctggcccagagtaatttaccactcccccgattatatgtatggtcagcttgaacagcgtaccaaatataaaagagagccagtatcattgaccctggcccttctactaggaggattaaccatgggagggattgcagctggaatagggacggggaccactgccttaattaaaacccagcagtttgagcagcttcatgccgctatccagacagacctcaacgaagtcgaaaagtcaattaccaacctagaaaagtcactgacctcgttgtctgaagtagtcctacagaaccgcagaggcctagatttgctattcctaaaggagggaggtctctgcgcagccctaaaagaagaatgttgtttttatgcagaccacacggggctagtgagagacagcatggccaaattaagagaaaggcttaatcagagacaaaaactatttgagacaggccaaggatggttcgaagggctgtttaatagatccccctggtttaccaccttaatctccaccatcatgggacctctaatagtactcttactgatcttactctttggaccttgcattctcaatcgattggtccaatttgttaaagacaggatctcagtggtccaggctctggttttgactcagcaatatcaccagctaaaacccatagagtacgagccatga

SEQ ID NO: 36 - protein, 654 aa, Murine leukemia virus
MARSTLSKPPQDKINPWKPLIVMGVLLGVGMAESPHQVFNVTWRVTNLMTGRTANATSLLGTVQDAFPKLYFDLCDLVGEEWDPSDQEPYVGYGCKYPAGRQRTRTFDFYVCPGHTVKSGCGGPGEGYCGKWGCETTGQAYWKPTSSWDLISLKRGNTPWDTGCSKVACGPCYDLSKVSNSFQGATRGGRCNPLVLEFTDAGKKANWDGPKSWGLRLYRTGTDPITMFSLTRQVLNVGPRVPIGPNPVLPDQRLPSSPIEIVPAPQPPSPLNTSYPPSTTSTPSTSPTSPSVPQPPPGTGDRLLALVKGAYQALNLTNPDKTQECWLCLVSGPPYYEGVAVVGTYTNHSTAPANCTATSQHKLTLSEVTGQGLCMGAVPKTHQALCNTTQSAGSGSYYLAAPAGTMWACSTGLTPCLSTTVLNLTTDYCVLVELWPRVIYHSPDYMYGQLEQRTKYKREPVSLTLALLLGGLTMGGIAAGIGTGTTALIKTQQFEQLHAAIQTDLNEVEKSITNLEKSLTSLSEVVLQNRRGLDLLFLKEGGLCAALKEECCFYADHTGLVRDSMAKLRERLNQRQKLFETGQGWFEGLFNRSPWFTTLISTIMGPLIVLLLILLFGPCILNRLVQFVKDRISVVQALVLTQQYHQLKPIEYEP

SEQ ID NO: 37 - DNA, 1938 bp, Murine leukemia virus
  source 1..1938: mol_type = genomic DNA
atggaaggtccagcgttctcaaaaccccttaaagataagattaacccgtggaagtccttaatggtcatgggggtctatttaagagtagggatggcagagagcccccatcaggtctttaatgtaacctggagagtcaccaacctgatgactgggcgtaccgccaatgccacctcccttttaggaactgtacaagatgccttcccaagattatattttgatctatgtgatctggtcggagaagagtgggacccttcagaccaggaaccatatgtcgggtatggctgcaaataccccggagggagaaagcggacccggacttttgacttttacgtgtgccctgggcataccgtaaaatcggggtgtggggggccaagagagggctactgtggtgaatggggttgtgaaaccaccggacaggcttactggaagcccacatcatcatgggacctaatctcccttaagcgcggtaacaccccctgggacacgggatgctccaaaatggcttgtggcccctgctacgacctctccaaagtatccaattccttccaaggggctactcgagggggcagatgcaaccctctagtcctagaattcactgatgcaggaaaaaaggctaattgggacgggcccaaatcgtggggactgagactgtaccggacaggaacagatcctattaccatgttctccctgacccgccaggtcctcaatatagggccccgcatccccattgggcctaatcccgtgatcactggtcaactacccccctcccgacccgtgcagatcaggctccccaggcctcctcagcctcctcctacaggcgcagcctctatagtccctgagactgccccaccttctcaacaacctgggacgggagacaggctgctaaacctggtagaaggagcctatcaggcgcttaacctcaccaatcccgacaagacccaagaatgttggctgtgcttagtgtcgggacctccttattacgaaggagtagcggtcgtgggcacttataccaatcattctaccgccccggccagctgtacggccacttcccaacataagcttaccctatctgaagtgacaggacagggcctatgcatgggagcactacctaaaactcaccaggccttatgtaacaccacccaaagtgccggctcaggatcctactaccttgcagcacccgctggaacaatgtgggcttgtagcactggattgactccctgcttgtccaccacgatgctcaatctaaccacagactattgtgtattagttgagctctggcccagaataatttaccactcccccgattatatgtatggtcagcttgaacagcgtaccaaatataagagggagccagtatcgttgaccctggcccttctgctaggaggattaaccatgggagggattgcagctggaatagggacggggaccactgccctaatcaaaacccagcagtttgagcagcttcacgccgctatccagacagacctcaacgaagtcgaaaaatcaattaccaacctagaaaagtcactgacctcgttgtctgaagtagtcctacagaaccgaagaggcctagatttgctcttcctaaaagagggaggtctctgcgcagccctaaaagaagaatgttgtttttatgcagaccacacgggactagtgagagacagcatggccaaactaagggaaaggcttaatcagagacaaaaactatttgagtcaggccaaggttggttcgaagggcagtttaatagatccccctggtttaccaccttaatctccaccatcatgggacctctaatagtactcttactgatcttactctttggaccctgcattctcaatcgattggtccaatttgttaaagacaggatctcagtggtccaggctctggttttgactcaacaatatcaccagctaaaacctatagagtacgagccatga

SEQ ID NO: 38 - protein, 645 aa, Murine leukemia virus
MEGPAFSKPLKDKINPWKSLMVMGVYLRVGMAESPHQVFNVTWRVTNLMTGRTANATSLLGTVQDAFPRLYFDLCDLVGEEWDPSDQEPYVGYGCKYPGGRKRTRTFDFYVCPGHTVKSGCGGPREGYCGEWGCETTGQAYWKPTSSWDLISLKRGNTPWDTGCSKMACGPCYDLSKVSNSFQGATRGGRCNPLVLEFTDAGKKANWDGPKSWGLRLYRTGTDPITMFSLTRQVLNIGPRIPIGPNPVITGQLPPSRPVQIRLPRPPQPPPTGAASIVPETAPPSQQPGTGDRLLNLVEGAYQALNLTNPDKTQECWLCLVSGPPYYEGVAVVGTYTNHSTAPASCTATSQHKLTLSEVTGQGLCMGALPKTHQALCNTTQSAGSGSYYLAAPAGTMWACSTGLTPCLSTTMLNLTTDYCVLVELWPRIIYHSPDYMYGQLEQRTKYKREPVSLTLALLLGGLTMGGIAAGIGTGTTALIKTQQFEQLHAAIQTDLNEVEKSITNLEKSLTSLSEVVLQNRRGLDLLFLKEGGLCAALKEECCFYADHTGLVRDSMAKLRERLNQRQKLFESGQGWFEGQFNRSPWFTTLISTIMGPLIVLLLILLFGPCILNRLVQFVKDRISVVQALVLTQQYHQLKPIEYEP

SEQ ID NO: 39 - DNA, 1590 bp, unidentified
  source 1..1590: mol_type = unassigned DNA
atggatctctttcccattttggtcgtggtgctcatgacagatactgtcttagggaagtttcaaattgtcttcccggatcagaatgaactggagtggagaccagttgtgggtgactctcggcattgcccacagtcatcagaaatgcaattcgatggaagcagatcccagaccatactgactgggaaagctcccgtggggatcacgccctctaaatcagatggatttatctgccatgccgcaaaatgggtgacaacatgtgatttcaggtggtatgggccgaaatacatcactcattcaatacatcatctgagaccgacaacatcagactgtgagacagctctccaaaggtataaagatgggagcttaatcaatcttggattccccccagaatcctgcggttatgcaacagtcacagattctgaggcaatgttggtccaagtgactccccaccacgttggggtggatgattatagaggtcactggatcgacccactatttccaggaggagaatgctccaccaatttttgtgatacagtccacaattcatcggtgtggatccccaagagtcaaaagactgacatctgtgcccagtctttcaaaaatatcaagatgaccgcatcttacccctcagaaggagcattggtgagtgacagatttgccttccacagtgcatatcatccaaatatgccggggtcaactgtttgcataatggacttttgcgaacaaaaggggttgagattcacaaatggagagtggatgggtctcaatgtggagcaatccatccgagagaagaagataagtgccatcttcccaaattgtgttgcagggactgaaatccgagccacactagaatcagaaggggcaagaactttgacgtgggagactcaaagaatgctagattactctttgtgtcagaacacctgggacaaagtttccaggaaagaacctctcagtccgcttgacttgagctatctgtcaccaagggctccagggaaaggcatggcctataccgtcataaacggaaccctgcattcggctcatgctaaatacattagaacctggattgattatggagaaatgaaggaaattaaaggtggacgtggagaatattccaaggctcctgagctcctctggtcccagtggttcgattttggaccgttcaaaattggaccgaatggactcctgcacacagggaaaacctttaaattccctctttatttgatcggagcaggcataattgacgaagatctgcatgaactagatgaggctgctcccattgatcacccacaaatgcctgacgcgaaaagcgttcttccagaagatgaagagatattcttcggagacacaggtgtatccaaaaaccctatcgagttgattcaaggatggttctcaaattggagagagagtgtaatggcaatagtcggaattgttctactcatcgttgtgacatttctggcgatcaagacggtccgggtgcttaattgtctctggagacccagaaagaaaagaatcgtcagacaagaagtagatgttgaatcccgactaaaccattttgagatgagaggctttcctgaatatgttaagagataa

SEQ ID NO: 40 - protein, 529 aa, unidentified
MDLFPILVVVLMTDTVLGKFQIVFPDQNELEWRPVVGDSRHCPQSSEMQFDGSRSQTILTGKAPVGITPSKSDGFICHAAKWVTTCDFRWYGPKYITHSIHHLRPTTSDCETALQRYKDGSLINLGFPPESCGYATVTDSEAMLVQVTPHHVGVDDYRGHWIDPLFPGGECSTNFCDTVHNSSVWIPKSQKTDICAQSFKNIKMTASYPSEGALVSDRFAFHSAYHPNMPGSTVCIMDFCEQKGLRFTNGEWMGLNVEQSIREKKISAIFPNCVAGTEIRATLESEGARTLTWETQRMLDYSLCQNTWDKVSRKEPLSPLDLSYLSPRAPGKGMAYTVINGTLHSAHAKYIRTWIDYGEMKEIKGGRGEYSKAPELLWSQWFDFGPFKIGPNGLLHTGKTFKFPLYLIGAGIIDEDLHELDEAAPIDHPQMPDAKSVLPEDEEIFFGDTGVSKNPIELIQGWFSNWRESVMAIVGIVLLIVVTFLAIKTVRVLNCLWRPRKKRIVRQEVDVESRLNHFEMRGFPEYVKR

SEQ ID NO: 41 - DNA, 22 bp, synthetic construct
  source 1..22: mol_type = other DNA
aaaagaaaaggggggactggaa